(19) United States
(12) Patent Application Publication
(10) Pub. No.: US 2026/0248858 A1
(43) Pub. Date: Aug. 27, 2026

(54) COMPOSITIONS AND METHODS COMPRISING TISSUE IMPLANTS

(71) Applicant: Ossium Health, Inc., San Francisco, CA (US)

(72) Inventors: Erik J. Woods, Carmel, IN (US); Wa’el Alrawashdeh, Zionsville, IN (US); Matthew Krach, Indianapolis, IN (US); Kimberly Buchanan, Indianapolis, IN (US)

(21) Appl. No.: 19/651,150

(22) Filed: Apr. 17, 2026

Related U.S. Application Data
(63) Continuation of application No. PCT/US2024/052039, filed on Oct. 18, 2024.
(60) Provisional application No. 63/592,110, filed on Oct. 20, 2023.
(60) Provisional application No. 63/648,406, filed on May 16, 2024.

(51) Int. Cl. A61K 35/32

(52) U.S. Cl. CPC A61K 35/32

(57) ABSTRACT

Provided herein are compositions that include bone grinding and a tissue sample derived from intervertebral disc. The compositions may include or be used as therapeutics. For example, the compositions may be implanted into a subject's body, such as in a body part, organ, or tissue to cause a therapeutic effect in the subject. In some embodiments, the compositions may be used for cell therapy. In some embodiments, the compositions may be used to treat cancer. In some embodiments, cells, such as stem cells and/or bone marrow cells in the compositions of the present disclosure may be used to treat diseases such as cancer.

### CROSS-REFERENCE TO RELATED APPLICATIONS

This application is a continuation of PCT Application No PCT/US2024/052039, filed Oct. 18, 2024, which claims priority to U.S. Provisional Patent Application No. 63/592,110, filed Oct. 20, 2023, and to U.S. Provisional Patent Application No. 63/648,406, filed May 16, 2024. The entire contents of each of the aforementioned patent applications are incorporated herein by reference.

### FIELD

Aspects of the present disclosure relate to compositions that include bone grinding and a tissue sample derived from intervertebral disc. In some embodiments, the compositions include or are used as a therapeutic. In some embodiments, the composition further includes cells such as stem cells and/or bone marrow cells. Also disclosed herein are methods of use of the present disclosure for the treatments of diseases including cancer.

### BACKGROUND

Cell therapies and tissue samples derived from animals and/or humans have vast therapeutic applications including repairing and reconstructing tissues or treating diseases such as cancer in live subjects. These may include tissue-derived samples that can be implanted in the body of a live human to achieve a therapeutic effect.

### SUMMARY

Samples derived from tissues can be processed and implanted into a human subject to achieve a therapeutic effect and treat a condition in the subject. In some embodiments, a condition to be treated in a subject includes a cancer, such as a hematologic cancer. In some embodiments, therapeutic cells, such as stem cells can be used to treat the hematologic cancer. Therapeutic cells or tissue-derived samples used for cell therapy may include bone cells and/or bone marrow cells. Provided herein are compositions which may have applications in cell therapy, as well as methods of making and using such compositions.

Accordingly, some embodiments provided herein relate to compositions. In some embodiments, the compositions include bone grinding and a tissue sample derived from intervertebral disc. In some embodiments, the tissue sample derived from intervertebral disc includes at least about 10% of the mass of the composition. In some embodiments, the tissue sample derived from intervertebral disc includes at least about 5%, at least about 6%, at least about 7%, at least about 8%, at least about 9%, at least about 10%, at least about 11%, at least about 12%, at least about 13%, at least about 15%, at least about 15%, at least about 16%, at least about 17%, at least about 18%, at least about 19%, at least about 20% or more of the mass of the composition.

In some embodiments, the bone grinding includes at least about 70%, at least about 75%, at least about 80% or more of the mass of the composition. In some embodiments, the tissue sample derived from intervertebral disc includes about 15% of the mass of the composition. In some embodiments, the tissue sample derived from intervertebral disc includes at most about 30%, at most about 25%, at most about 20%, at most about 19%, at most about 18%, at most about 17%, at most about 16%, at most t about 15%, at most about 14%, at most about 13%, at most about 12%, at most about 11%, at most about 10% or less of the mass of the composition.

In some embodiments, the bone grinding includes at least about 70% of the mass of the composition. In some embodiments, the bone grinding includes at least 80% of the mass of the composition. In some embodiments, the bone grinding includes about 85% of the mass of the composition. In some embodiments, the bone grinding includes at least about 50%, at least about 55%, at least about 60%, at least about 65%, at least about 70%, at least about 75%, at least about 80%, at least about 85%, at least about 90% or more of the mass of the composition. In some embodiments, the bone grinding includes at most about 90%, at most about 85%, at most about 80%, at most about 75% of the mass of the composition or less.

In some embodiments, the tissue sample derived from intervertebral disc includes at least about 10% of the mass of the composition, and bone grinding includes at least about 70% of the mass of the composition. In some embodiments, the tissue sample derived from intervertebral disc includes about 15% of the mass of the composition, and the bone grinding includes about 85% of the mass of the composition. In some embodiments, a mass ratio of the tissue sample derived from the intervertebral disc to that of the bone grinding is from about 0.17 to about 0.18. In some embodiments, the tissue sample derived from intervertebral disc confers structural integrity to the composition. In some embodiments, the tissue sample derived from intervertebral disc includes collagen.

In some embodiments, the structural integrity of the composition is higher than the structural integrity of a similar composition which contains bone grinding but does not contain a significant amount of a tissue sample derived from intervertebral disc. In some embodiments, the structural integrity is characterized by measuring a scatter distance in a drop test.

In some embodiments, a scatter distance of the composition is at most about 20 centimeters (cm), at most about 18 cm, at most about 15 cm, at most about 10 cm, at most about 5 cm, at most about 3 cm, at most about 2 cm or less. In some embodiments, the sample includes a substantial structural integrity characterized by measuring a scatter distance in a drop test.

In some embodiments, the composition includes bone cells and disc cells. In some embodiments, the composition further includes cell growth media. In some embodiments, the composition further includes an electrolyte solution. In some embodiments, the electrolyte solution is provided at a 1:4 ratio relative to the tissue sample derived from intervertebral disc. In some embodiments, the electrolyte solution includes or is Plasmalyte.

In some embodiments, the composition further includes a tissue homogenizer. In some embodiments, the composition further includes a tissue sample derived from intervertebral disc that is substantially homogenized. In some embodiments, the composition is substantially homogenized. In some embodiments, the composition further includes a cryopreservative. In some embodiments, the cryopreservative includes dimethyl sulfoxide (DMSO), propylene glycol (PG), ice recrystallization inhibitor (IRI), or any combination thereof. In some embodiments, the cryopreservative is present in an amount of at least about 1%, at least about 2%, at least about 3%, at least about 5%, at least about 8%, at least about 10% or more.

Some embodiments provided herein relate to therapeutics. In some embodiments, the therapeutics include any of the compositions provided herein. In some embodiments, the therapeutics are intended for use in cell therapy. In some embodiments, the therapeutics are an implantable therapeutic configured to be implanted in a body of a patient, and the composition acts as a matrix for constructing the therapeutic.

Some embodiments provided herein relate to methods of treating a bone defect using any of the compositions or the therapeutics described herein. In some embodiments, the methods include implanting the compositions or an object made of the compositions in a subject and/or in a tissue or body part thereof. In some embodiments, the subject is a human. In some embodiments, the method is performed to treat cancer in the subject. In some embodiments, the method includes performing cell therapy on the subject via the cells in the composition.

Some embodiments provided herein relate to kits that include any of the compositions described herein and instructions for performing any of the methods described herein. Some embodiments provided herein relate to kits that include any of the therapeutics described herein and instructions for performing any of the methods described herein. In some embodiments, the composition and/or the therapeutic is provided in a vial which may be a part of the kit. In some embodiments, the kit further includes a tool for implanting the composition into a body of a subject for cell therapy. In some embodiments, the composition is stored/preserved at a temperature of at most about −50 degrees Celsius (° C.), at most about −60° C., at most about −70° C., at most about −80° C., at most about −85° C., at most about −100° C., at most about −150° C. or lower.

In some embodiments, the composition further includes one or more antibiotics, antimycotics, or both. In some embodiments, the antibiotic and/or antimycotic contribute to the preservation of the composition. In some embodiments, the composition includes a mixture or cocktail of one or more antibiotics and/or antimycotics with an orthogonal kill profile.

In some embodiments, the composition further comprises at least one of BMP-2, BMP-4, BMP-7, BMP-9, FGF1, FGF2, osteopontin, osteoactivin, VEGF, TNFa, PDGF-BB, or any combination thereof. In some embodiments, BMP-2 is present in at least about 30, 75, 100, or 120 pg per gram of tissue. In some embodiments, BMP-4 is present in at least about 0.5, 1, 5, 10, 25, 50, 100, 500, or 1000 pg per gram of tissue. In some embodiments, BMP-7 is present in at least 1500, 1600, 1800, 2000, 2300, 2500, 3000, or 3500 pg per gram of tissue. In some embodiments, BMP-9 is present in at least about 0.5, 0.75, 1, 1.25, 1.5, 1.75, 2, 2.25, or 2.5 pg per gram of tissue. In some embodiments, FGF1 is present in at least about 250, 300, 350, 400, 450, or 500 pg per gram of tissue. In some embodiments, FGF2 is present in at least about 1, 3, 5, 7, 9, or 10 pg per gram of tissue. In some embodiments, osteopontin is present in at least about 500, 1000, 2000, 4000, 5000, or 7500 pg per gram of tissue. In some embodiments, osteoactivin is present in at least about 1500, 2000, 3000, 4000, or 5000 pg per gram of tissue. In some embodiments, VEGF is present in at least about 5000, 10000, 15000, 20000, or 25000 pg per gram of tissue. In some embodiments, TNFa is present in at least about 2, 3, 4, 5, 6, or 7 pg per gram of tissue. In some embodiments, PDFG-BB is present in at least about 50, 75, 100, 125, 150, 175, or 200 pg per gram of tissue.

Some embodiments provided herein relate to methods of extracting a cell from a tissue. In some embodiments, the methods include recovering a tissue from an organ or from a body of a subject under aseptic conditions, thereby generating a recovered tissue. In some embodiments, recovering under aseptic conditions includes: (i) placing the recovered tissue in a sterile container and hydrating the recovered tissue with an electrolyte solution; (ii) homogenizing the recovered tissue using a homogenizing solution; (iii) slicing and grinding the recovered tissue in presence of treatment media including an antibiotic and antimycotic cocktail with orthogonal kill profile and a cryopreservative solution; and/or (iv) rinsing the antibiotic and antimycotic cocktail with the cryopreservative solution. In some embodiments, the treatment media is present before, during, and/or after the slicing and grinding. In some embodiments, the method further includes mixing or rotating the sample in the container.

In some embodiments, the methods further include extracting the cell from the recovered tissue, thereby generating an extracted cell and/or storing the extracted cell in presence of the cryopreservative, in a temperature at which biological activity in the cell is minimized, and the cell is preserved long term. In some embodiments, the cell is provided in any of the compositions described herein.

In some embodiments, the organ/tissue from which the cell is derived includes or is intervertebral disc of an animal, such as a mammal, such as a human. In some embodiments, the method further includes assessing the organ/tissue (e.g., disc) in terms of potential degeneration, and selecting non-degenerated organ/tissue (e.g., disc) for performing the method thereon and extracting the cell therefrom. In some embodiments, the slicing and grinding generates a plurality of tissue pieces. In some embodiments, each of the plurality of tissue pieces is smaller than about 4 centimeters (cm), about 3 cm, about 2 cm, about 1 cm, about 0.5 cm or smaller.

In some embodiments, the electrolyte solution is provided in a mass ratio of about 1:4 relative to the mass of the tissue. In some embodiments, the electrolyte solution is a solution sold under brand name Plasmalyte. In some embodiments, the homogenizer solution includes a solution sold under brand name ProScientific PRO250.

### BRIEF DESCRIPTION OF THE DRAWINGS

In addition to the features described above, additional features and variations will be readily apparent from the descriptions of the drawings and embodiments provided herein. It is to be understood that these drawings depict embodiments and are not intended to be limiting in scope.

FIG. 1 shows a drop test apparatus for testing and measuring the structural integrity of the compositions of the present disclosure via debris count and furthest scatter distance metrics.

FIGS. 2A-2B provide example images for counted debris and particles scattered to furthest distance.

FIGS. 3A-3E provide example images of drop test of embodiments of a composition described herein that includes intervertebral disc demonstrating superior structural integrity and coherence compared to similar compositions which do not include intervertebral disc.

FIGS. 4A-4D provide example images of drop test of a composition which does not include intervertebral disc and which scatters into debris upon performing the drop test thereon, showing lower structural coherence and integrity compared to the composition that includes intervertebral disc as shown in FIGS. 3A-3E.

FIG. 5 shows a one-way ANOVA of viable cell counts for three benchtop stability donors at one-hour intervals post-thaw.

FIG. 6 shows a non-limiting example schematic for the production process of embodiments of the compositions described herein.

FIGS. 7A-7B show non-limiting example graph for the total number of viable cells (FIG. 7A) and viable osteoblasts (FIG. 7B) in various cryopreservative agents at Time Zero, Post-Thaw per 5 g of embodiments of the compositions described herein, following treatment with different cryopreservative solutions.

FIGS. 8A-8E shows a non-limiting example graph for the total number of viable cells in various cryopreservative agents at one month, Post-Thaw, for total number of cells per 5 g of embodiments of the compositions described herein (FIG. 8A) total number of cells per 5 g bone allograft (FIG. 8B), total viable osteoblasts per 5 g bone allograft (FIG. 8C), total viable MSC per 5 g bone allograft (FIG. 8D), and number of colonies per 5 g bone allograft (FIG. 8E), following treatment with different cryopreservative solutions.

### DETAILED DESCRIPTION

Described herein are compositions of viable bone matrix, and methods of making and using the compositions. More particularly, in some embodiments, the compositions include bone grindings and intervertebral disc matrix.

Bone allografts are used in a variety of orthopedic and podiatric surgical applications to help promote new bone formation. Viable Bone Matrix (VBX) products are a particularly effective bone allograft that have viable cells native to the tissue. VBX or Cellular Bone Matrix (CBM) uses the bone grinding by-product from clinical hematopoietic progenitor cells (HPC), and marrow production into a clinical VBX product, thus maximizing the potential of the donated tissue. Embodiments described herein relate to compositions having improved characteristics, including improved handling and moldability. In some embodiments, the compositions include bone grindings and intervertebral discs. The intervertebral discs undergo additional processing steps before being combined with the bone grindings.

In some embodiments, the compositions described herein have increased stability for a reasonable storage duration at supra-cryogenic temperatures (e.g., −80° C.) as well as being resistant to transient warming events (TWE). TWE are most likely to occur with the end user, such as when the product is unpacked and temporarily stored in a −80° C. freezer until use and/or when transported from the freezer to the operating room. To accomplish this, the impact of ice recrystallization must be managed, which is not currently addressed through cryoprotective additives, such as dimethyl sulfoxide (DMSO) alone.

It is well established that potentially deleterious ice recrystallization is a thermodynamically-driven process that favors the formation of larger ice crystals via the transfer of water molecules from one crystal to another through the bulk water phase. Inhibition of ice recrystallization is achieved in nature through glycopeptides known as biological antifreezes. These antifreeze glycoproteins (AFGP) were first discovered in Antarctic fish in the 1950s and have subsequently been reported in other animals, insects, and fish that inhabit sub-zero environments. These AFGP, or ice recrystallization inhibitors (IRI), can bind to the surface of ice crystals and selectively reduce the rapidly developing needle-shaped ice growth that can occur during cryopreservation of cells due to ice binding (Alasmar et al., 2023; McMunn et al., 2024). However, these natural AFGPs are not effective cryoprotectants as ice-binding modifies the crystal habit resulting in increased cellular damage at cryogenic temperatures.

In contrast, small molecule carbohydrate based IRIs have been chemically synthesized using GMP approaches suitable for use in clinical products and are now commercially available (for example, through PanTHERA CryoSolutions). These molecules have been extensively studied from in vitro/in vivo toxicological and efficacy perspectives.

In some embodiments, the composition described herein uses fresh bone grindings (after bone marrow elution) from milled vertebral bones. In some embodiments, the bone grindings are re-milled to a smaller size than standard VBX products and combined with fresh intervertebral discs that would otherwise be discarded during tissue debridement. In some embodiments, the compositions include a cryopreserved viable bone allograft product with improved handling and moldability. In some embodiments, all sources of fungal or bacterial contamination are eliminated during processing. In addition, any immunogenic reaction potential is removed by minimizing the number of reactive immunogenic cells. In some embodiments, native bone cells are prepared for long-term frozen storage.

Compositions including tissue-derived samples have vast applications for cell therapy including for cancer treatment. In some embodiments, such samples may include stem cells, bone marrow cells, bone grinding, additional components, and combinations thereof. In some embodiments, such compositions may be implanted into a subject or a body part thereof to generate a therapeutic effect. In some embodiments, such compositions may include a therapeutic or may be a therapeutic. The compositions may be preserved in low temperatures to minimize biological activity therein. The compositions may be homogenized, may include electrolytes, antibiotics, antimycotics, or any combination thereof to improve and preserve their properties. The components of the compositions may be adjusted to optimize their structural coherence and integrity and make them more suitable for implantation into the human body and enhance their therapeutic effects. In some embodiments, such compositions may be provided as part of a kit including instructions for use, such as to perform the methods detailed in the present disclosure.

Also provided herein is a composition including bone grinding and a tissue sample derived from intervertebral disc. In some embodiments, the tissue sample derived from intervertebral disc includes at least about 10% of the mass of the composition. In some embodiments, the tissue sample derived from intervertebral disc includes at least about 5%, at least about 6%, at least about 7%, at least about 8%, at least about 9%, at least about 10%, at least about 11%, at least about 12%, at least about 13%, at least about 15%, at least about 15%, at least about 16%, at least about 17%, at least about 18%, at least about 19%, at least about 20% or more of the mass of the composition.

In some embodiments, the bone grinding includes at least about 70%, at least about 75%, at least about 80% or more of the mass of the composition. In some embodiments, the tissue sample derived from intervertebral disc includes about 15% of the mass of the composition. In some embodiments, the tissue sample derived from intervertebral disc includes at most about 30%, at most about 25%, at most about 20%, at most about 19%, at most about 18%, at most about 17%, at most about 16%, at most t about 15%, at most about 14%, at most about 13%, at most about 12%, at most about 11%, at most about 10% or less of the mass of the composition.

In some embodiments, the bone grinding includes at least about 70% of the mass of the composition. In some embodiments, the bone grinding includes at least 80% of the mass of the composition. In some embodiments, the bone grinding includes about 85% of the mass of the composition. In some embodiments, the bone grinding includes at least about 50%, at least about 55%, at least about 60%, at least about 65%, at least about 70%, at least about 75%, at least about 80%, at least about 85%, at least about 90% or more of the mass of the composition. In some embodiments, the bone grinding includes at most about 90%, at most about 85%, at most about 80%, at most about 75% of the mass of the composition or less.

In some embodiments, the tissue sample derived from intervertebral disc includes at least about 10% of the mass of the composition, and bone grinding includes at least about 70% of the mass of the composition. In some embodiments, the tissue sample derived from intervertebral disc includes about 15% of the mass of the composition, and the bone grinding includes about 85% of the mass of the composition. In some embodiments, a mass ratio of the tissue sample derived from the intervertebral disc to that of the bone grinding is from about 0.17 to about 0.18. In some embodiments, the tissue sample derived from intervertebral disc confers structural integrity to the composition. In some embodiments, the tissue sample derived from intervertebral disc includes collagen.

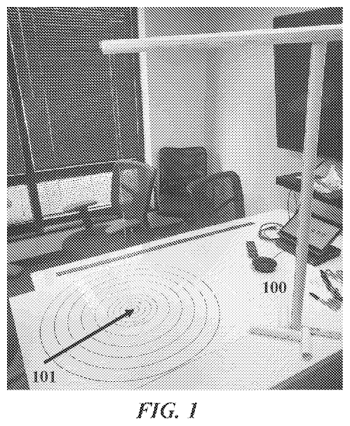

In some embodiments, the structural integrity of the composition is higher than the structural integrity of a similar composition which contains bone grinding but does not contain a significant amount of a tissue sample derived from intervertebral disc. In some embodiments, the structural integrity is characterized by measuring a scatter distance in a drop test. Examples 1-2 and FIGS. 1, 2A-2B, 3A-3E, and 4A-4D demonstrate this point.

In some embodiments, a scatter distance of the composition is at most about 20 centimeters (cm), at most about 18 cm, at most about 15 cm, at most about 10 cm, at most about 5 cm, at most about 3 cm, at most about 2 cm or less. In some embodiments, the sample includes a substantial structural integrity characterized by measuring a scatter distance in a drop test. cm the scatter distance of the composition is at most about 20 centimeters (cm), at most about 18 cm, at most about 15 cm, at most about 10 cm, at most about 5 cm, at most about 3 cm, at most about 2 cm or less.

In some embodiments, the composition includes bone cells and disc cells. In some embodiments, the composition further includes cell growth media. In some embodiments, the composition further includes an electrolyte solution. In some embodiments, the electrolyte solution is provided at a 1:4 ratio relative to the tissue sample derived from intervertebral disc. In some embodiments, the electrolyte solution includes or is Plasmalyte.

In some embodiments, the composition further includes a tissue homogenizer. In some embodiments, the composition further includes the tissue sample derived from intervertebral disc is substantially homogenized. In some embodiments, the composition is substantially homogenized. In some embodiments, the composition further includes a cryoprotectant. In some embodiments, the composition further includes a dimethyl sulfoxide (DMSO), propylene glycol (PG), ice recrystallization inhibitor (IRI), or any combination thereof. In some embodiments, the composition includes at least about 1%, at least about 2%, at least about 3%, at least about 5%, at least about 8%, at least about 10% or more DMSO.

Also provided herein is a therapeutic composition including the composition of any one of the preceding embodiments. In some embodiments, the therapeutic is intended for use in cell therapy. In some embodiments, the composition of the present disclosure acts as a matrix to build a cell therapy product thereon. The therapeutic or cell therapy product may be an implantable product made of the composition of the present disclosure (e.g., a composition including bone grinding and intervertebral disc as described throughout the disclosure). The composition may be configured for implantation into the body of a subject.

Also provided herein is a method of treating a bone defect using the composition or the therapeutic of any one of the preceding embodiments. The method includes implanting the composition or an object made of the composition in a subject and/or in a tissue or body part thereof. In some embodiments, the subject is a human. In some embodiments, the method is performed to treat cancer in the subject. In some embodiments, the method includes performing cell therapy on the subject via the cells in the composition.

Also provided herein is a kit including the composition and instructions for performing the method. Also provided herein is a kit that includes the therapeutic and instructions for performing the method. In some embodiments, the composition and/or the therapeutic is provided in a vial which may be a part of the kit. In some embodiments, the kit further includes a tool for implanting the composition into a body of a subject for cell therapy. In some embodiments, the composition is stored/preserved at a temperature of at most about −50 degrees Celsius (° C.), at most about −60° C., at most about −70° C., at most about −80° C., at most about −85° C., at most about −100° C., at most about −150° C. or lower.

In some embodiments, the composition further includes one or more antibiotics, antimycotics, or both. In some embodiments, the antibiotic and/or antimycotic contribute to the preservation of the composition. In some embodiments, the composition includes a mixture or cocktail of one or more antibiotics and/or antimycotics with an orthogonal kill profile.

In some embodiments, the composition further comprises at least one of: BMP-2, BMP-4, BMP-7, BMP-9, FGF1, FGF2, osteopontin, osteoactivin, VEGF, TNFa, PDGF-BB, or any combination thereof. In some embodiments, BMP-2 is present in an amount ranging from about 30 to about 120 pg/gram of tissue, for example, at least about 30, 75, 100, or 120 pg per gram of tissue, or an amount within a range defined by any two of the aforementioned values. In some embodiments, BMP-4 is present in an amount ranging from about 0.5 to about 1,000 pg/gram of tissue, for example, at least about 0.5, 1, 5, 10, 25, 50, 100, 500, or 1000 pg per gram of tissue, or an amount within a range defined by any two of the aforementioned value. In some embodiments, BMP-7 is present in an amount ranging from about 1500 to about 3500 pg/gram of tissue, for example, at least 1500, 1600, 1800, 2000, 2300, 2500, 3000, or 3500 pg per gram of tissue, or an amount within a range defined by any two of the aforementioned value. In some embodiments, BMP-9 is present in an amount of about 0.5 to about 2.5 pg/gram of tissue, for example, at least about 0.5, 0.75, 1, 1.25, 1.5, 1.75, 2, 2.25, or 2.5 pg per gram of tissue, or an amount within a range defined by any two of the aforementioned value. In some embodiments, FGF1 is present in an amount of about 250 to about 500 pg/gram of tissue, for example, at least about 250, 300, 350, 400, 450, or 500 pg per gram of tissue, or an amount within a range defined by any two of the aforementioned value. In some embodiments, FGF2 is present in an amount of about 1 to about 10 pg/gram of tissue, for example, at least about 1, 3, 5, 7, 9, or 10 pg per gram of tissue, or an amount within a range defined by any two of the aforementioned value. In some embodiments, osteopontin is present in an amount ranging from about 500 to about 7500 pg/gram of tissue, for example, at least about 500, 1000, 2000, 4000, 5000, or 7500 pg per gram of tissue, or an amount within a range defined by any two of the aforementioned value. In some embodiments, osteoactivin is present in an amount of about 1500 to about 5000 pg/gram of tissue, for example, at least about 1500, 2000, 3000, 4000, or 5000 pg per gram of tissue, or an amount within a range defined by any two of the aforementioned value. In some embodiments, VEGF is present in an amount of about 5000 to about 25000 pg/gram of tissue, for example, at least about 5000, 10000, 15000, 20000, or 25000 pg per gram of tissue, or an amount within a range defined by any two of the aforementioned value. In some embodiments, TNFa is present in an amount of about 2 to about 7 pg/gram of tissue, for example, at least about 2, 3, 4, 5, 6, or 7 pg per gram of tissue, or an amount within a range defined by any two of the aforementioned value. In some embodiments, PDFG-BB is present in an amount of about 50 to about 200 pg/gram of tissue, for example, at least about 50, 75, 100, 125, 150, 175, or 200 pg per gram of tissue, or an amount within a range defined by any two of the aforementioned value.

Also provided herein is a method of extracting a cell from a tissue such as a disc, the method including: (a) recovering a tissue from an organ of from a body of a subject under aseptic conditions, thereby generating a recovered tissue, wherein recovering under aseptic conditions includes: (i) placing the recovered tissue in a sterile container and hydrating the recovered tissue with an electrolyte solution; (ii) homogenizing the recovered tissue using a homogenizing solution; (iii) slicing and grinding the recovered tissue in presence of treatment media including an antibiotic and antimycotic cocktail with orthogonal kill profile and a cryoprotectant solution, wherein the treatment media is present before, during, and/or after the slicing and grinding; and (iv) rinsing the antibiotic and antimycotic cocktail with the cryoprotectant solution. (a) includes mixing or rotating the sample in the container. In some embodiments, the tissue includes or is disc. In some embodiments, the tissue (e.g., disc) is initially processed in a laboratory blender to generate a homogenate. In some embodiments, the tissue (e.g., disc) is processed using a homogenizer.

The method further includes (b) extracting the cell from the recovered tissue, thereby generating an extracted cell; and (c) storing the extracted cell in presence of the cryoprotectant, in a temperature at which biological activity in the cell is minimized, and the cell is preserved for the long term. In some embodiments, the cell is provided in a composition provided anywhere in the present disclosure.

In some embodiments, the tissue from which the cell is derived includes or is intervertebral disc of an animal, such as a mammal, such as a human. In some embodiments, the method may further include assessing the tissue (e.g., disc) in terms of potential degeneration, and selecting non-degenerated disc for performing the method thereon and extracting the cell therefrom. In some embodiments, the slicing and grinding generates a plurality of tissue pieces. In some embodiments, each of the plurality of tissue pieces is smaller than about 4 centimeters (cm), about 3 cm, about 2 cm, about 1 cm, about 0.5 cm or smaller.

In some embodiments, the electrolyte solution is provided in a mass ratio of about 1:4 relative to the mass of the tissue. In some embodiments, the electrolyte solution is a solution sold under brand name Plasmalyte. In some embodiments, the homogenizer solution includes a solution sold under brand name ProScientific PRO250.

Some embodiments provided herein relate to making the compositions described herein. In some embodiments, the methods include several steps, including (i) marrow production, (ii) invertebrate disc preparation, (iii) bone grinding, and/or (iv) tissue combination. Detailed descriptions of non-limiting example methodologies for each step follow.

### HPC, Marrow Production Steps

Debridement & Decontamination: Vertebral bodies, sourced from deceased organ donors, are debrided to remove the surrounding tissue and pedicles. The intervertebral discs are cut away from the vertebral bodies and set aside for production of the compositions. The isolated vertebral bodies undergo decontamination, which includes treatment with sodium hypochlorite solution and hydrogen peroxide solution.

Grind: Decontaminated vertebral bodies are turned into bone grindings containing bone marrow using a bone mill and Grind Medium.

Bone Marrow Extraction: The bone grindings containing bone marrow undergo a series of three washes to elute off the bone marrow from the bone grindings. The grindings are agitated on a shaker while submerged in Grind Medium once (1000 mL) and Rinse Medium twice (500 mL each), decanting in between each wash to separate the bone marrow from the bone grindings. The bone grindings are set aside for VBX production.

### Intervertebral Disc Production

Inspection: The intervertebral discs set aside from debridement of the vertebral bodies are inspected for degeneration, cartilaginous endplates, and other debris. If any of these are observed, the affected discs or sections of discs are removed from continued processing.

Decontamination: The remaining discs, with a total mass between (or equal to) 40 and 100 grams, are submerged in 300 mL of 70% isopropyl alcohol (IPA) and agitated on a shaker for 15 minutes. The 70% IPA is then decanted from the discs.

Washes: After decontamination, the discs are washed three times, each time with 300 mL of Plasma-Lyte A. Each wash is agitated on a shaker and then the Plasma-Lyte A is decanted. The first and second washes are agitated for 1 minute, while the third wash is agitated for 5 minutes.

Homogenization: After decanting the final Plasma-Lyte A wash, fresh Plasma-Lyte A is added to the discs at a volume of 1.5 mL per gram of disc. To the now combined discs and Plasma-Lyte A, an equal volume of 20% DMSO Wash Medium is added relative to the combined mass and volume of discs and Plasma-Lyte A (1 g=1 mL). Thus, the final mixture of discs, Plasma-Lyte A, and 20% DMSO Wash Medium is 20% discs by weight and 10% DMSO. The disc mixture is then homogenized via a heavy-duty blender by repeating the following blend sequence three times: low speed for 30 seconds, medium speed for 30 seconds, high speed for 90 seconds, then rest for 30 to 60 seconds. If after the three blend cycles, any disc pieces larger than 0.5 cm remain, they are removed from continued processing. Finally, the disc suspension container is surrounded by ice packs to expedite cooling the disc suspension and set aside until needed later in the process.

### Bone Grindings

Regrind: The bone grindings set aside after bone marrow extraction, with a total mass of 500 g or less, are reground to a finer grind size using a Fortios Bone Mill with XXXF milling drum and Rinse Medium (an equal volume to the mass of bone grindings).

Treatment with Antibiotics/Antimycotic: After being reground, the bone grindings are submerged in an equal volume, relative to the mass of bone grindings, of Treatment Medium containing antibiotics/antimycotic (250 mg Vancomycin, 50 mg Voriconazole, 50 mg Polymyxin B, and 50 mg Gentamicin). The bone grindings in Treatment Medium are agitated on a shaker for 60 minutes. In some embodiments, a sterile magnet is added during the shaking step to remove metal particulate that may be present from the grinding process. The Treatment Medium is then decanted from the bone grindings.

Washes: The bone grindings are then washed five times. Each wash uses twice the volume of Plasma-Lyte A relative to the mass of bone grindings, which is decanted after each wash. The first, second, third, and fifth washes are agitated on a shaker for 1 minute, while the fourth wash is agitated on a shaker for 20 minutes.

### Combined Tissues

Combination of Tissues: Once both the disc suspension (cooled to ≤25° C.) and reground bone grindings are ready, they are combined to yield 85% bone grindings and 15% intervertebral discs by mass.

Treatment with Cryoprotectant: To the combined disc suspension and bone grindings, an equal volume of 10% DMSO Wash Medium containing IRI is added relative to the mass of combined tissue. The combined tissue in 10% DMSO Wash Medium is agitated on a shaker for 10 minutes. After the 10-minute cryoprotectant treatment, the combined tissue in solution is centrifuged at 1000×g for 5 minutes and the solution is decanted.

Through collaboration with PanTHERA CryoSolutions, the IRI manufacturer, and development testing, an IRI concentration of 2.86 mg/mL in the Treatment Medium was established. For example, to achieve this concentration, 2.99 g of IRI is reconstituted in 115 mL of 90% DMSO. Then, 110 mL of this IRI in 90% DMSO (2.86 g IRI) is combined with 890 mL of Plasma-Lyte A to yield 1 L of Treatment Medium. This IRI concentration gets diluted to 2 mg/mL in the final cryoprotective solution due to diluting the Treatment Medium 1:1 with the combined tissue, which is 37.5% liquid (10% DMSO) via the disc suspension.

Homogenization: After decanting the cryoprotectant solution, the tissue is mixed for at least 3 minutes with a high torque stirrer (set at 1000 RPM) until homogenous, generating the final tissue product.

Package & Freeze: The final tissue product is aliquoted by mass, based on a density of 1.3 gram per cc, into product packaging to yield 2.5 cc, 3 cc, 5 cc, 10 cc, and/or 15 cc units. The units are then transferred into an ultra-low temperature freezer (≤−70° C.), arranged flat in a single layer directly on the freezer shelves. The final product units are frozen for a minimum of 12 hours prior to being relocated for long-term frozen storage.

### Terms

In the present disclosure, reference is made to the accompanying drawings, which form a part hereof. In the drawings, similar symbols typically identify similar components, unless context dictates otherwise. The illustrative embodiments described in this disclosure, including the drawings and claims, are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Unless defined otherwise, all technical and scientific terms used herein have the same meaning as is commonly understood by one of ordinary skill in the art to which the disclosed subject matter belongs when read in light of the current disclosure.

The section headings used herein are for organizational purposes only and are not to be construed as limiting the subject matter described.

The articles “a” and “an” are used herein to refer to one or to more than one (for example, at least one) of the grammatical object of the article, unless the context dictates otherwise. By way of example, “an element” means one element or more than one element.

By “about” is meant a quantity, level, value, number, frequency, percentage, dimension, size, amount, weight or length that is approximately the recited value. Where it is not clear from the context what is encompassed by “about,” it will mean the value recited +/−10%.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The terms “individual”, “subject”, or “patient” as used herein have their plain and ordinary meaning as understood in light of the specification, and mean a human or a non-human mammal, e.g., a dog, a cat, a mouse, a rat, a cow, a sheep, a pig, a goat, a non-human primate, or a bird, e.g., a chicken, as well as any other vertebrate or invertebrate. The term “mammal” is used in its usual biological sense. Thus, it includes, but is not limited to, primates, including simians (chimpanzees, apes, monkeys) and humans, cattle, horses, sheep, goats, swine, rabbits, dogs, cats, rodents, rats, mice, guinea pigs, or the like.

As used herein, the term “isolated” has its plain and ordinary meaning as understood in light of the specification, and refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from equal to, about, at least, at least about, not more than, or not more than about, 10%, about 20%, about 30%, about 40%, about 50%, about 60%, about 70%, about 80%, about 90%, about 95%, about 98%, about 99%, substantially 100%, or 100% of the other components with which they were initially associated (or ranges including and/or spanning the aforementioned values). In some embodiments, isolated agents are, are about, are at least, are at least about, are not more than, or are not more than about 80%, about 85%, about 90%, about 91%, about 92%, about 93%, about 94%, about 95%, about 96%, about 97%, about 98%, about 99%, substantially 100%, or 100% pure (or ranges including and/or spanning the aforementioned values). As used herein, a substance that is “isolated” may be “pure” (e.g., substantially free of other components). As used herein, the term “isolated cell” may refer to a cell not contained in a multi-cellular organism or tissue.

As used herein, “in vivo” has its plain and ordinary meaning as understood in light of the specification and refers to the performance of a method inside living organisms, usually animals, mammals, including humans, and plants, as opposed to a tissue extract or dead organism.

As used herein, “ex vivo” has its plain and ordinary meaning as understood in light of the specification and refers to the performance of a method outside a living organism with little alteration of natural conditions.

As used herein, “in vitro” has its plain and ordinary meaning as understood in light of the specification and refers to the performance of a method outside of biological conditions, e.g., in a petri dish or test tube.

As used herein, the term “allograft” has its plain and ordinary meaning as understood in light of the specification, and refers to a tissue, organ, or cell graft from a donor of the same species as the recipient that is not genetically identical.

As used herein, the term “annulus fibrosus (AF)” has its plain and ordinary meaning as understood in light of the specification, and refers to the peripheral region of the intervertebral disc, consisting mostly of collagen fibers that create concentric rings of rigid fibrils that hold the nucleus pulposus in place.

As used herein, the term “bone grinding” has its plain and ordinary meaning as understood in light of the specification, and refers to the substance including bone generated during the HPC, Marrow production process; grindings are produced from debrided, decontaminated vertebral bodies passed through a bone mill, and then eluted of bone marrow in accordance with the HPC, Marrow filtration process.

As used herein, the term “calcein” has its plain and ordinary meaning as understood in light of the specification, and refers to a cell permanent dye that fluoresces at a specific wavelength when metabolized by living cells.

As used herein, the term “cartilaginous endplate” has its plain and ordinary meaning as understood in light of the specification, and refers to the superior and inferior regions of the intervertebral disc, consisting of firm hyaline cartilage connecting the disc to the vertebral bodies above and below the disc.

As used herein, the term “cluster of differentiation (CD)” has its plain and ordinary meaning as understood in light of the specification, and refers to the protocol used for the identification and investigation of cell surface molecules providing targets for immunophenotyping of cells.

As used herein, the term “carboxyfluorescein succinimidyl ester (CFSE)” has its plain and ordinary meaning as understood in light of the specification, and refers to a stain that covalently binds to intracellular proteins and emits a wavelength in the FITC channel after it is metabolized. It is commonly used to track generations of cell proliferation, as each new generation in a culture contains half of the stain, and thus shows lower emission than the previous generation on flow cytometry.

As used herein, the term “cryoprotective agent (CPA)” has its plain and ordinary meaning as understood in light of the specification, and refers to any molecule or composition that protects a substance from damage, deterioration, or otherwise harm associated with freezing. In some embodiments, the CPA protects from short-term damage. In some embodiments, the CPA protects from long-term damage.

As used herein, the term “dexamethasone” has its plain and ordinary meaning as understood in light of the specification, and refers to a small molecule drug intended to decrease lymphocyte colony proliferation.

As used herein, the term “gentamicin” has its plain and ordinary meaning as understood in light of the specification, and refers to a broad-spectrum antibiotic.

As used herein, the term “nucleus pulposus (NP)” has its plain and ordinary meaning as understood in light of the specification, and refers to the central region of the intervertebral disc, consisting of a gelatinous proteoglycan rich matrix designed to provide flexibility and absorb compression forces.

As used herein, the term “Polymyxin B” has its plain and ordinary meaning as understood in light of the specification, and refers to an antibiotic with unique mechanism of action against bacterial cell membranes; particularly efficacious against resistant Gram-negative bacteria.

As used herein, the term “Rinse Media” has its plain and ordinary meaning as understood in light of the specification, and refers to media created for multiple production and manufacturing processes, including Plasma-Lyte A with 2.5% HSA, which serves to optimize pH, osmolarity, and osmolality for cells of interest during processing.

As used herein, the term “Stemulate hPL” has its plain and ordinary meaning as understood in light of the specification, and refers to Human Platelet Lysate manufactured by BioLife Solutions; used in this context to bind collagenase and protease to stop the primary digest reaction that breaks down extracellular bone matrix for cell viability testing.

As used herein, the term “vancomycin” has its plain and ordinary meaning as understood in light of the specification, and refers to a broad spectrum antibiotic with efficacy against resistant Gram-positive bacteria.

As used herein, the term “voriconazole” has its plain and ordinary meaning as understood in light of the specification, and refers to a broad-spectrum antifungal.

As used herein, the term “Grind Medium” has its plain and ordinary meaning as understood in light of the specification, and refers to Plasma-Lyte A with 2.5% HSA and 10 U/mL Benzonase.

As used herein, the term “Ice Recrystallization Inhibitor (IRI)” has its plain and ordinary meaning as understood in light of the specification, and refers to a small molecule glycoprotein compound that controls ice crystal size and growth during cryopreservation and transient warming events (TWE).

As used herein, the term “treatment medium” has its plain and ordinary meaning as understood in light of the specification, and refers to Plasma-Lyte A with 2.5% HSA, 0.5 mg/mL Vancomycin, 0.1 mg/mL Voriconazole, 0.1 mg/mL Polymyxin B, and 0.1 mg/mL Gentamicin.

As used herein, the term “transient warming event (TWE)” has its plain and ordinary meaning as understood in light of the specification, and refers to a situation in which a cryopreserved sample expriences a sudden, short-term increase in temperature, such as when a sample is packaged/unpackaged during distribution.

As used herein, the term “ultra-low temperature freezer” has its plain and ordinary meaning as understood in light of the specification, and refers to a mechanical freezer capable of maintaining a temperature of ≤−70° C., used for the freezing and storage of VBX.

As used herein, the term “10% DMSO Wash medium” has its plain and ordinary meaning as understood in light of the specification, and refers to Plasma-Lyte A with 10% DMSO and 2.86 mg/mL IRI.

As used herein, the term “20% DMSO Wash medium” has its plain and ordinary meaning as understood in light of the specification, and refers to Plasma-Lyte A with 20% DMSO.

AFGP: Antifreeze Glycoproteins

B/F: Bacteriostasis/Fungistasis sterility test used to assess whether a test sample is inhibitory to the growth of microorganisms

CBM: Cellular Bone Matrix; the former name for VBX

DMSO: Dimethyl Sulfoxide

EBR: Electronic Batch Record

GMP: Good Manufacturing Practice

HSA: Human Serum Albumin

IFU: Instructions For Use

IPA: isopropyl alcohol

IRI: Ice Recrystallization Inhibitors

IVD: intervertebral discs

LN2: Liquid Nitrogen

MLR: Mixed Lymphocyte Reaction

MSC: Mesenchymal Stem/Stromal Cells

MSC Culture Medium: MSC Growth Medium with 10% Human Platelet Lysate, 2 ng/mL EGF, and 2 ng/ml FGF2

OB: Osteoblasts

OPO: Organ Procurement Organization

PBMC: Peripheral Blood Mobilized Cells

QC: quality control

RPM: revolutions-per-minute

RPMI-FBS: A cell culture medium containing fetal bovine serum (FBS)

VBX: Viable Bone Matrix; a cryopreserved viable bone allograft (formerly referred to as Cellular Bone Matrix, CBM).

Some embodiments provided herein are described by way of the following numbered alternatives:
1. A composition comprising bone grinding and a tissue sample derived from intervertebral disc, wherein the tissue sample derived from intervertebral disc comprises at least about 10% of the mass of the composition.
2. The composition of alternative 1, wherein the tissue sample derived from intervertebral disc comprises about 15% of the mass of the composition.
3. The composition of alternative 1 or 2, wherein the bone grinding comprises at least about 70% of the mass of the composition.
4. The composition of any one of alternatives 1-3, wherein the bone grinding comprises at least about 80% or more of the mass of the composition.
5. The composition of any one of alternatives 1-4, wherein the bone grinding comprises about 85% of the mass of the composition.
6. The composition of any one of alternatives 1-5, wherein the tissue sample derived from intervertebral disc comprises about 15% of the mass of the composition, and the bone grinding comprises about 85% of the mass of the composition.
7. The composition of any one of alternatives 1-6, wherein a mass ratio of the tissue sample derived from intervertebral disc to that of the bone grinding is from about 0.17 to about 0.18.
8. The composition of any one of the preceding alternatives, wherein the tissue sample derived from intervertebral disc confers structural integrity to the composition.
9. The composition of any one of the preceding alternatives, wherein the tissue sample derived from intervertebral disc comprises collagen.
10. The composition of alternative 8 or 9, wherein the structural integrity of the composition is higher than the structural integrity of a similar composition which contains bone grinding but does not contain a significant amount of a tissue sample derived from intervertebral disc.
11. The composition of any one of alternatives 8-10, wherein the structural integrity is characterized by measuring a scatter distance in a drop test.
12. The composition of alternative 11, wherein a scatter distance of the composition is at most about 20, at most about 18, at most about 15, at most about 10, at most about 5, at most about 3, at most about 2 centimeters (cm) or less.
13. The composition of alternative 11 or 12, wherein the sample comprises a substantial structural integrity characterized by measuring a scatter distance in a drop test.
14. The composition of alternative 13, wherein the scatter distance of the composition is at most about 20, at most about 18, at most about 15, at most about 10, at most about 5, at most about 3, at most about 2 centimeters (cm) or less.
15. The composition of any one of the preceding alternatives, wherein the composition comprises bone cells and disc cells.
16. The composition of alternative 15, further comprising cell growth media.
17. The composition of any one of the preceding alternatives, further comprising an electrolyte solution.
18. The composition of alternative 17, wherein the electrolyte solution is provided at a 1:4 ratio relative to the tissue sample derived from intervertebral disc.
19. The composition of alternative 17 or 18, wherein the electrolyte solution comprises or is Plasmalyte.
20. The composition of any one of the preceding alternatives, further comprising a tissue homogenizer.
21. The composition of any one of the preceding alternatives, wherein the tissue sample derived from intervertebral disc is substantially homogenized.
22. The composition of any one of the preceding alternatives, wherein the composition is substantially homogenized.
23. The composition of any one of the preceding alternatives, further comprising a cryoprotectant.
24. The composition of any one of the preceding alternatives, further comprising dimethyl sulfoxide (DMSO), propylene glycol (PG), ice recrystallization inhibitor (IRI), or any combination thereof.
25. The composition of alternative 24, wherein the composition comprises at least about 1%, at least about 2%, at least about 3%, at least about 5%, at least about 8%, at least about 10% or more DMSO.
26. A therapeutic comprising the composition of any one of the preceding alternatives.
27. The therapeutic of alternative 26 intended for use in cell therapy.
28. The therapeutic of alternative 26, wherein the therapeutic is an implantable therapeutic configured to be implanted in a body of a patient, and wherein the composition acts as a matrix for constructing the implantable therapeutic.
29. A method of treating a bone defect using the composition or the therapeutic of any one of the preceding alternatives.
30. The method of alternative 29, wherein the method comprises implanting the composition or an object made of the composition in a tissue of a subject.
31. The method of alternative 30, wherein the subject is a human.
32. The method of any one of alternatives 29-31, wherein the method is performed to heal or alleviate the symptoms of a bone defect in the subject.
33. The method of any one of the alternatives 29-32, comprising performing cell therapy on the subject via the cells in the composition of any one of the alternatives 1-25.
34. A kit comprising the composition of any one of the alternatives 1-25 and instructions for performing the method of any one the alternatives 29-33.
35. A kit comprising the therapeutic of any one of the alternatives 26 or 27 and instructions for performing the method of any one the alternatives 29-33.
36. The kit of alternative 34 or 35, wherein the composition or the therapeutic is provided in a vial.
37. The kit of alternative 34 or 35, further comprising a tool for implanting the composition into a body of a subject for cell therapy.
38. The composition of any one of the preceding alternatives preserved at a temperature of at most about −50 degrees Celsius (C), at most about −60° C., at most about −70° C., at most about −80° C., at most about −84° C., at most about −85° C., at most about −86° C., at most about −100° C., at most about −150° C. or lower.
39. The composition of any one of the preceding alternatives, further comprising one or more antibiotics or antimycotics.
40. The composition of alternative 39, wherein the antibiotic or antimycotic contributes to the preservation of the composition.
41. The composition of any one of alternatives 38-40, wherein the antibiotics or antimycotics are part of a mixture or cocktail with an orthogonal kill profile.
42. The composition of any one of alternatives 1-41, further comprising at least one of: BMP-2, BMP-4, BMP-7, BMP-9, FGF1, FGF2, osteopontin, osteoactivin, VEGF, TNFa, PDGF-BB, or any combination thereof.
43. The composition of alternative 42, wherein BMP-2 is present in at least about 30, 75, 100, or 120 pg per gram of tissue.
44. The composition of alternative 42 or 43, wherein BMP-4 is present in at least about 0.5, 1, 5, 10, 25, 50, 100, 500, or 1000 pg per gram of tissue.
45. The composition of any one of alternatives 42-44, wherein BMP-7 is present in at least 1500, 1600, 1800, 2000, 2300, 2500, 3000, or 3500 pg per gram of tissue.
46. The composition of any one of alternatives 42-45, wherein BMP-9 is present in at least about 0.5, 0.75, 1, 1.25, 1.5, 1.75, 2, 2.25, or 2.5 pg per gram of tissue.
47. The composition of any one of alternatives 42-46, wherein FGF1 is present in at least about 250, 300, 350, 400, 450, or 500 pg per gram of tissue.
48. The composition of any one of alternatives 42-47, wherein FGF2 is present in at least about 1, 3, 5, 7, 9, or 10 pg per gram of tissue.
49. The composition of any one of alternatives 42-48, wherein osteopontin is present in at least about 500, 1000, 2000, 4000, 5000, or 7500 pg per gram of tissue.
50. The composition of any one of alternatives 42-49, wherein osteoactivin is present in at least about 1500, 2000, 3000, 4000, or 5000 pg per gram of tissue.
51. The composition of any one of alternatives 42-50, wherein VEGF is present in at least about 5000, 10000, 15000, 20000, or 25000 pg per gram of tissue.
52. The composition of any one of alternatives 42-51, wherein TNFa is present in at least about 2, 3, 4, 5, 6, or 7 pg per gram of tissue.
53. The composition of any one of alternatives 42-52, wherein PDFG-BB is present in at least about 50, 75, 100, 125, 150, 175, or 200 pg per gram of tissue.
54. A method of extracting a cell from a tissue, the method comprising:
   recovering a tissue from a body of a subject under aseptic conditions, thereby generating a recovered tissue, wherein recovering under aseptic conditions comprises:
   placing the recovered tissue in a sterile container and hydrating the tissue with an electrolyte solution;
   homogenizing the tissue using a homogenizing solution;
   slicing and grinding the tissue in presence of treatment media comprising a cocktail of antibiotics and antimycotics with orthogonal kill profile and a cryoprotectant solution, wherein the treatment media is present before, during, or after the slicing and grinding;
   rinsing the cocktail with the cryoprotectant solution;
   extracting the cell from the recovered tissue, thereby generating an extracted cell; and
   storing the extracted cell in presence of the cryoprotectant, in a temperature at which biological activity in the cell is minimized, and the cell is preserved for the long term.
55. The method of alternative 54, wherein the tissue comprises or is intervertebral disc of a mammal.
56. The method of alternative 54 or 55, further comprising assessing the tissue in terms of potential degeneration, and optionally selecting non-degenerated tissue for performing the method thereon.
57. The method of any one of alternatives 54-56, wherein the slicing and grinding generates a plurality of tissue pieces.
58. The method of any one of alternatives 54-57, wherein each of the plurality of tissue pieces is smaller than about 4, 3, 2, 1, 0.5 centimeters (cm) or smaller.
59. The method of any one of alternatives 54-58, wherein the electrolyte solution is provided in a mass ratio of about 1:4 relative to the mass of the tissue.
60. The method of any one of alternatives 54-59, wherein the electrolyte solution is a solution sold under brand name Plasmalyte.
61. The method of any one of alternatives 54-60, wherein the homogenizer solution comprises a solution sold under brand name ProScientific PRO250.
62. The method of any one of alternatives 54-61, wherein recovering comprises mixing or rotating the sample in the container.
63. A method of preparing an intervertebral disc suspension, the method comprising:
   removing a vertebral disc from a spine;
   weighing the vertebral disc, wherein the vertebral disc is about 40, 45, 50, 55, 60, 65, 70, 75, 80, 85, 90, 95, 100, or any integer that is between 40 and 100, grams in weight;
   decontaminating the vertebral disc; and
   blending the vertebral disc with a Plasma-Lyte solution to create a vertebral disc suspension.
64. The method of alternative 63, wherein the decontamination step comprises administering an antibiotic and/or antimycotic agent.
65. The method of alternative 63 or 64, wherein the decontamination step comprises administering isopropyl alcohol (IPA).
66. The method of alternative 65, wherein the decontamination step comprises administering a solution of 70% IPA.
67. The method of any one of alternatives 63-66, wherein the decontamination step is about 10, 11, 12, 13, 14, 15, 16, 17, 18, 19, 20, 21, 22, 23, 24, 25, 26, 27, 28, 29, 30, or any integer that is between 10 and 30, minutes in length.
68. The method of alternative 67, wherein the decontamination step is about 15 minutes in length.
69. The method of any one of alternatives 63-68, wherein decontamination is performed while agitating/shaking the invertebrate disc.
70. The method of any one of alternatives 63-69, further comprises 1, 2, 3, 4, or 5 washes between decontamination and blending.
71. The method of alternative 70, wherein the 1, 2, 3, 4, or 5 washes is about 0.5, 1, 2, 3, 4, 5, 6, 7, 8, 9, 10, or any integer that is between 0.5 and 10, minutes in length.
72. The method of alternative 70 or 71, wherein the 1, 2, 3, 4, or 5 washes comprise administering Plasma-Lyte A.
73. The method of any one of alternatives 70-72, wherein the 1, 2, 3, 4, or 5 washes are performed while agitating/shaking the invertebrate disc.
74. The method of any one of alternatives 72-73, wherein the Plasma-Lyte A is removed through decanting.
75. The method of any one of alternatives 63-74, wherein the Plasma-Lyte solution comprises about 0.5, 1, 1.5, 2, 2.4, 5, or any integer between 0.5 and 5, mL of Plasma-Lyte A per gram of vertebral disc.
76. The method of alternative 75, wherein the Plasma-Lyte solution comprises 1.5 mL Plasma-Lyte A per gram of vertebral disc.
77. The method of any one of alternatives 63-76, wherein the vertebral disc solution is generated through use of a blender.
78. The method of any one of alternatives 63-77, wherein the vertebral disc solution comprises a final concentration of about 10, 15, 20, 25, 30, 40, or any integer between 10 and 40, percent disc by weight.
79. The method of alternative 78, wherein the vertebral disc solution comprises a final concentration of about 20% disc by weight.
80. The method of any one of alternatives 63-79, wherein the vertebral disc solution comprises a final concentration of 5, 10, 15, 20, 25, 30, 40, or any integer between 4 and 40, percent DMSO.
81. The method of alternative 70, wherein the vertebral disc solution comprises a final concentration of 10% DMSO.
82. The method of any one of alternatives 63-79, wherein the vertebral disc solution does not comprise any vertebral disc components at least 0.25, 0.5, 1, 2, 3, or any integer between 0.25 and 3, cm in diameter.
83. The method of alternative 82, wherein the vertebral disc solution does not comprise any vertebral disc components at least 0.5 cm in diameter.
84. A method of preparing a ground bone composition, the method comprising:
   isolating a vertebral body comprising bone marrow from a spine;
   decontaminating the vertebral body;
   grinding the vertebral body into a bone grinding;
   removing the bone marrow from the bone grinding;
   regrinding the bone grinding;
   treating the bone grinding with an antibiotic and/or an antimycotic agent;
   washing the bone grinding with a wash buffer, and removing the wash buffer to result in a final ground bone composition.
85. The method of alternative 84, wherein decontamination comprises administering sodium hypochlorite solution and/or hydrogen peroxide.
86. The method of alternative 84 or 85, wherein grinding is performed using a grind mill.
87. The method of any one of alternatives 84-86, wherein grinding further comprises administering a Grind Medium.
88. The method of alternative 87, wherein the Grind Medium comprises Plasma-Lyte A, HSA, and/or benzonase.
89. The method of alternative 88, wherein HSA is at 0.5, 1, 1.5, 2, 2.5, 3, 3.5, 4, 4.5, 5, or any integer that is between 0.5 and 5 percent.
90. The method of alternative 89, wherein HSA is at 2.5%.
91. The method of any one of alternatives 88-90, wherein benzonase is at 1, 5, 6, 7, 8, 9, 10, 11, 12, 13, 14, 15, 20, 25, or any integer that is between 1 and 25, U/mL.
92. The method of alternative 91, wherein benzonase is at 10 U/mL.
93. The method of any one of alternatives 84-92, wherein the XXF bone grinding comprises bone that is at most 1, 2, 3, 4, 5, or any integer between 1 and 5, mm in diameter.
94. The method of any one of alternatives 84-93, wherein the bone marrow extraction comprises at least 1, 2, 3, 4, or 5 washes of the XXF bone grinding.
95. The method of alternative 94, wherein the XXF bone grinding is washed 3 times.
96. The method of any one of alternatives 94-95, wherein the 1, 2, 3, 4, or 5 washes are performed while agitating/shaking the XXF bone grinding.
97. The method of any one of alternatives 94-96, wherein the marrow and wash is separated from the bone griding by decanting.
98. The method of any one of alternatives 94-97, wherein at least the first wash comprises administering the Grind Medium of any one of alternatives 76-80.
99. The method of any one of alternatives 94-98, wherein at least the second, third, fourth, fifth, or final wash comprises administering a Rinse Medium.
100. The method of alternative 99, wherein the Rinse Medium comprises Plasma-Lyte A.
101. The method of alternative 98 or 99, wherein the Rinse Medium comprises a final concentration of 0.5, 1, 1.5, 2, 2.5, 5, 10, 15, 20, 25, or any integer between 0.5 and 25, percent HSA.
102. The method of alternative 100, wherein the Rinse Medium comprises 2.5% HSA.
103. The method of any one of alternatives 84-102 wherein the XXF bone grinding following bone marrow extraction has a total weight that is not more than 300, 400, 500, 600, 700, or any integer between 300 to 700, grams.
104. The method of alternative 103, wherein the XXF bone grinding has a total weight that is less than 500 grams following bone marrow extraction.
105. The method of any one of alternatives 84-104, wherein the regrinding is performed with a Fortios Bone Mill and/or a XXXF milling drum.
106. The method of any one of alternatives 84-105, wherein the regrinding further comprises administering the Rinse Medium of any one of alternatives 100-102.
107. The method of any one of alternatives 84-106, wherein the XXXF bone grinding comprises bone that is at most 0.1, 0.2, 0.4, 0.6, 0.8, 1, 2, 3, 4, 5, or any integer between 0.1 and 5, mm in diameter.
108. The method of any one of alternatives 84-107, wherein the treatment with an antibiotic and/or an antimycotic is at least 0.5, 0.75, 1, 1.5, 2, or any integer between 0.5 and 2, hours long.
109. The method of any one of alternatives 84-108, wherein the treatment with an antibiotic and/or an antimycotic is performed while shaking/agitating the XXXF bone grinding.
110. The method of any one of alternatives 84-109, wherein the antibiotic and/or antimycotic is removed from solution through decanting.
111. The method of any one of alternatives 84-110, wherein the antibiotic and/or antimycotic is administered to the XXXF bone grinding along with the Rinse Medium of any one of alternatives 100-102.
112. The method of any one of alternatives 84-111, wherein the antibiotic and/or antimycotic is selected from the list consisting of: Vancomycin, Voriconazole, Polymyxin B, and Gentamicin, or any combination thereof.
113. The method of any one of alternatives 84-112, wherein washing the XXXF bone grinding in wash buffer is repeated at least once, twice, three, four, or five times.
114. The method of alternative 113, wherein each wash buffer is administered for at least 0.5, 1, 2, 3, 4, 5, 6, 7, 8, 9, 10, 15, 20, or any integer between 0.5 and 20, minutes.
115. The method of any one of alternatives 84-114, wherein the wash buffer is removed via decanting.
116. The method of any one of alternatives 84-115, wherein the wash buffer comprises Plasma-Lyte A.
117. A method of producing a bone tissue product, the method comprising:
   combining the intervertebral disc suspension of any one of alternatives 63-83 with the ground bone composition of any one of alternatives 84-116;
   treating the combination with a cryoprotectant agent;
   removing any supernatant from the combination; and
   drying the combination, resulting in the bone tissue product.
118. The method of alternative 117, wherein the intervertebral disc suspension is cooled prior to combining with the ground bone composition.
119. The method of alternative 118, wherein the intervertebral disc suspension is cooled to not more than 0, 5, 10, 15, 20, or 25° C.
120. The method of any one of alternatives 117-119, wherein the ground bone is about 50, 55, 60, 65, 70, 765, 80, 85, 90, or any integer between 50 and 90, percent of the combination by mass.
121. The method of any one of alternatives 117-120, wherein the combination comprises about 85% ground bone composition, and about 15% intervertebral disc suspension by mass.
122. The method of any one of alternatives 117-121, wherein the combination is treated with the cryoprotectant for at least 1, 5, 6, 7, 8, 9, 10, 11, 12, 13, 14, 15, 20, 25, 30, or any integer between 1 and 30, minutes.
123. The method of any one of alternatives 117-122, wherein the combination is treated with the cryoprotectant while shaken/agitated.
124. The method of any one of alternatives 117-123, wherein treatment with the cryoprotectant comprises administering 10% DMSO Wash Medium.
125. The method of any one of alternatives 117-124, wherein the cryoprotectant is an ice recrystallization inhibitor (IRI).
126. The method of any one of alternatives 117-125, wherein the supernatant is removed through centrifugation followed by decanting.
127. A method of storing the bone tissue product of any one of alternatives 105-114, the method comprising freezing the bone tissue product.
128. The method of alternative 127, wherein the bone tissue product is stored in a −80° C. freezer.
129. The method of alternative 127-128, wherein the bone tissue product is stored in a sealed, sterile package.

### Examples

The following illustrative examples are representative of embodiments of the methods and compositions described herein and are not meant to be limiting in any way.

### Example 1: Tissue Processing to Generate Implants

Described herein is an example protocol for tissue processing to yield an implant. In some embodiments, the tissue being processed can vertebral discs. In some embodiments, the tissues being processed can be vertebral discs and bones.

### A) Disc Homogenization

1) Whole intervertebral discs sliced from between vertebral bodies during debridement.
2) Discs assessed for degeneration. Degeneration, cartilaginous endplate, and other debris cut from full discs. [Note: This might be done instead using the mini tissue blender from Xiogenix]
3) Discs sliced into pieces between 0.5 and 1.0 cm in diameter using meat shearers.
4) Sliced discs weighed out.
5) Discs added to a sterile jar with Plasmalyte at a 1:4 disc to Plasmalyte ratio by mass.
6) Discs homogenized in the jar using ProScientific PRO250 tissue homogenizer, with 20 mm head, at 5000 RPM for 3 minutes or until all disc chunks are homogenized. This step requires rotating the jar under the homogenizer head slowly.

### B) Bone Grinding:

1) Post-filtration XXF bone grindings scooped into Fortios Bone Mill with XXXF blade head.
2) Bone grindings milled to XXXF size, while being washed through the bone mill using 500 mL Rinse Media.
3) XXXF bone grindings supernatant decanted.
4) XXXF bone grindings weighed out.

### C) Combining Discs and Bone Grindings

1. Enough suspended disc added to the sterile bowl of bone grindings to create a final product with 15% disc and 85% bone grindings by mass.
   i) The amount of disc needed equals [mass of bone grinding]*[0.15/0.85].
   ii) Pure disc accounts for 20% of disc suspension, so the final mass of suspension needed is 5× the amount of disc from Step 1i.
   iii) Final equation comes out to [mass of disc suspension]=[mass of bone grinding]*[15/17].
2. Sterile bowl containing disc suspension and bone grindings placed on shaker for 5 minutes at 150 RPM.
3. 500 mL of Treatment media containing antibiotics/antimycotics and 10% DMSO in Rinse Media added to bone grindings and disc.
4. Sterile bowl with media, disc suspension, and bone grindings placed on shaker at 150 RPM for 1 hour.
5. After 1 hour, media, disc suspension, and bone grindings poured into multiple 500 mL conical tubes.
6. Conical tubes centrifuged at 1000×g for 10 minutes.
7. Supernatant decanted from tubes.
8. VBX pulled from conical tubes using forceps and placed in a fresh sterile bowl. 500 mL of Plasmalyte with 10% DMSO poured into bowl. VBX and media swirled by hand for 1 minute. VBX and media poured into fresh 500 ml conical tubes. Conical tubes centrifuged at 1000×g for 5 minutes. Supernatant decanted from tubes.
9. Repeat Step 8 two more times.
10. Final product VBX Gen. 2 pulled from tubes and combined in a fresh sterile bowl.
11. VBX Gen. 2 aliquoted into pouches/vials and placed in the −86° C. freezer.

### Example 2: Assessment of Structural Integrity of Implants

The scope of this experiment encompassed the testing of the structural integrity of implants generated following the sample protocol in Example 1.

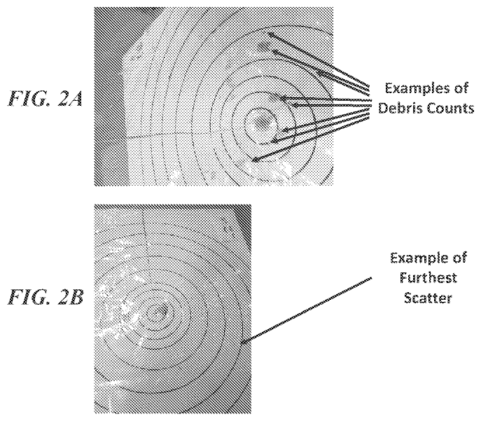

In order to test the structural integrity of implants were measured out into 5 gram balls and dropped from a predetermined height onto a target spot 101 on a flat surface using an apparatus 100 shown in FIG. 1. As shown in FIGS. 2A and 2B, the furthest radius of scatter and the number of debris particles were quantified. Higher structural integrity is evidenced by lower radius of scatter and fewer particles of debris.

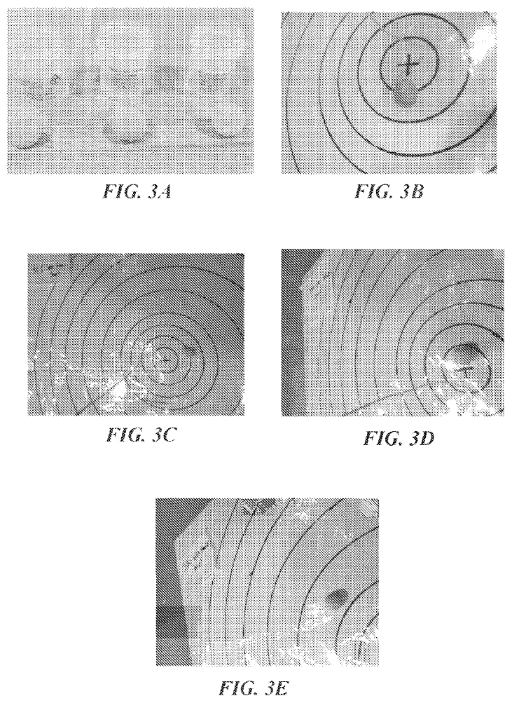

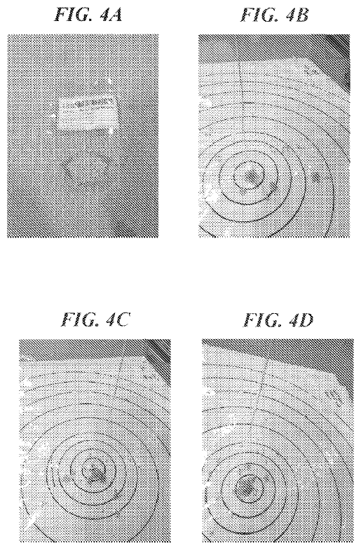

Structural integrities of compositions including bone grinding and 15-20% intervertebral discs were tested. Images of these tests are shown in FIGS. 3A-3E. Similar tests were performed for samples not including any intervertebral disc, and the results are shown in FIGS. 4A-4D.

FIG. 4A shows the composition not including intervertebral disc prior to being dropped. As shown in this image, even before dropping, this composition does not show structural coherence and integrity, compared to samples including intervertebral disc shown in FIG. 3A.

Table 1 summarize the furthest scatter distance (cm) and debris counts for a composition including bone grinding but not intervertebral disc.

TABLE 1
Structural Integrity of Bone Grindings with No Vertebral Disc Suspension
Bone Grindings Alone

| | Vertebral Disc | | Furthest | Debris |
|---|---|---|---|---|
| Donor ID | Suspension Ratio | Drop # | Scatter (cm) | Count |
| W23-269 | 0% | 1 | 15 | 14 |
| W23-269 | 0% | 2 | 20 | 31 |
| W23-269 | 0% | 3 | 20 | 35 |

| | | |
|---|---|---|
| AVERAGE | 18.3 | 26.7 |

Structural integrities of compositions including bone grinding with various ratios of vertebral disc suspension ratios were tested. These ratios included 10%, 15%, and 20% vertebral disc suspension. As shown in Table 2, implants with 10% vertebral disc suspension demonstrated reduced particle debris compared to bone grindings alone, despite a similar scatter radius. However, implants with 15% or 20% vertebral disc suspension demonstrated no scatter and no debris following the drop. This clearly demonstrates high structural integrity of implants Generated using the methods described herein. The images shown in FIGS. 3A-3E illustrate the same point. The composition balls shown in FIG. 3A, even before dropping show better structural integrity and coherence compared to the composition not including any intervertebral disc (shown in FIG. 4A).

TABLE 2
Structural Integrity of Bone Grindings with Vertebral Disc Suspension
Bone Grindings with Vertebral Disc Suspension

| | Vertebral Disc | | Furthest | Debris |
|---|---|---|---|---|
| Donor ID | Suspension Ratio | Drop # | Scatter (cm) | Count |
| W23-269 | 10% | 1 | 2.5 | 3 |
| W23-269 | 10% | 2 | 30 | 4 |
| W23-269 | 10% | 3 | 20 | 6 |
| AVERAGE | | | 17.5 | 4.3 |
| W23-269 | 15% | 1 | 0 | 0 |
| W23-269 | 15% | 2 | 0 | 0 |
| W23-269 | 15% | 3 | 0 | 0 |
| AVERAGE | | | 0 | 0 |
| W23-315 | 15% | 1 | 0 | 0 |
| W23-315 | 15% | 2 | 0 | 0 |
| W23-315 | 15% | 3 | 0 | 0 |
| AVERAGE | | | 0 | 0 |
| W23-330 | 15% | 1 | 0 | 0 |
| W23-330 | 15% | 2 | 0 | 0 |
| W23-330 | 15% | 3 | 0 | 0 |
| AVERAGE | | | 0 | 0 |
| W23-333 | 15% | 1 | 0 | 0 |
| W23-333 | 15% | 2 | 0 | 0 |
| W23-333 | 15% | 3 | 0 | 0 |
| AVERAGE | | | 0 | 0 |
| W23-269 | 20% | 1 | 0 | 0 |
| W23-269 | 20% | 2 | 0 | 0 |
| W23-269 | 20% | 3 | 0 | 0 |

| | | |
|---|---|---|
| AVERAGE | 0 | 0 |

### Example 3: Production of Graft Compositions

Orthopedic surgeons and other professionals using bone allograft products desire a product with improved cohesion and moldability. Competitors' products utilize demineralized cortical fibers tangled within the bone grindings to improve the cohesiveness of their products. To replicate and even exceed the cohesiveness of other competitors' bone allograft products, without the need for sourcing cortical bone and processing it to demineralized fibers, a product that combines the vertebral bone grindings and the intervertebral discs was proposed.

Three donors were processed for proof-of-concept testing (Table 3). The cohesiveness of each group tested was subjectively analyzed by observing particulates or chunks of the product flaking off while compressing and rolling the product in the hands. Each group was also analyzed by dropping the product onto a lab table covered in a sterile table cover, to observe how the product held up against impact forces.

TABLE 3
Adding Discs to Bone Grindings

| Study | | | | Experimental |
|---|---|---|---|---|
| # | Donor ID | Tested Variables | Experimental Groups | Outcomes |
| 1 | W437523000174 | 1. Bone Grinding vs. | A. XXF Bone Grinding Control | Product containing |
| | | Bone Grindings + | B. XXXF Bone Grinding Control | disc was the most |
| | | Disc | C. XXXF Bone and Disc | cohesive |
| | | | combined (15% Disc) | |
| 2 | W437523000178 | 1. Bone Grindings vs. | A. XXF Bone Grinding Control | Product with 15% |
| | | Bone Grindings + | B. XXXF Bone and Disc | disc was the most |
| | | Disc | combined (5% Disc) | cohesive, followed |
| | | 2. Disc Concentration | C. XXXF Bone and Disc | by 25% disc, then |
| | | in Final Product | combined (15% Disc) | 5% disc |
| | | | D. XXXF Bone and Disc | |
| | | | combined (25% Disc) | |
| 3 | W437523000185 | 1. Bone Grindings vs. | A. XXF Bone Grinding Control | Product containing |
| | | Bone Grindings + | B. XXXF Bone Grinding Control | disc was the most |
| | | Disc | C. XXXF Bone and Disc | cohesive |
| | | 2. Disc Concentration | combined (15% Disc) | |
| | | in Final Product | | |

The first donor spine tested (W437523000174) was divided in two. Half of the spine was debrided, decontaminated, and milled (via the Fortios Bone Mill) with the vertebral discs still attached to the vertebral bodies. This created a viscous solution during rinsing and filtration that prevented Production personnel from being able to properly rinse out bone marrow cells and decant Rinse Media since the ground vertebral discs retained a significant amount of the media. A portion of this material was then squeezed to remove the fluid and tested for cohesiveness in comparison to bone grindings milled with the XXF blade (1-3 mm grind size) and XXXF blade (0.5-1 mm grind size). The product exhibited good handling. It was also evident that future processing tests would need to be done with the bone and discs separated during the first bone milling with the XXF blade, so that the bone marrow could be rinsed out of the product for continued HPC, Marrow processing. The subsequently washed XXF bone grindings would then be ground again with vertebral discs using the XXXF milling drum.

The next donor spine that was used for proof-of-concept testing (W437523000178) was processed with the discs separated from the vertebral bodies. The vertebral bodies were then ground, and the bone marrow was eluted. The vertebral discs were then added to the bone grindings prior to being placed through the bone mill to produce a smaller grind size. This donor was processed with four groups: an XXF control with just bone grindings and three groups of bone grindings combined with a specific amount of sliced disc material. Each of the disc groups had 100 grams of total material. The first group contained 95 g of bone grindings and 5 g of vertebral disc; the second group contained 85 g of bone grindings and 15 g of vertebral disc; and the third group contained 75 g of bone grindings and 25 g of vertebral disc. These groups were each washed twice after grinding to remove excess bone marrow cells. During cohesiveness testing, it was clear that the group with 85 g of bone grindings and 15 g of vertebral disc had the best handling. The group with 5 g of disc was not quite as cohesive as the 15 g group, while the group with 25 g of disc was slimy and difficult to wash. Samples from each group were frozen in a −86° C. freezer, followed by cryopreservation in the LN2 cryo-tank. Upon thawing, each of the samples demonstrated similar cohesiveness to the fresh samples from the same groups. All three disc groups were difficult to re-grind as the bone grindings were originally made using the CCF grinder. It took more than 30 minutes to re-grind each of the groups (over 1.5 hours total to process all three disc groups), which would cause a significant increase in the processing time. It was concluded that grindings made with the XXF blade, instead of the CCF grinder, would be more rapidly reground by the XXXF blade.

The third donor spine that was used for proof-of-concept testing (W437523000185) was processed with three groups. The first group was an XXF bone grindings control, the second group was XXXF reground bone grindings, and the third group consisted of 42.5 g of XXF bone grindings with 7.5 g of sliced vertebral disc that was reground using the XXXF blade (15% disc). All three groups went through a treatment soak for 60 minutes followed by three washes in Plasma-Lyte A with 10% DMSO. Two samples from each group were frozen in a −86° C. freezer, followed by cryopreservation in the LN2 cryo-tank. The remainder of the fresh samples were tested for cohesiveness. The product containing 15% disc had the highest level of cohesiveness when analyzing both the fresh and post-thaw samples. The XXXF re-grind took less than two minutes for the 50 g sample of XXF bone grindings and disc, which was much faster than the previous donor that used CCF bone grindings.

### Example 4: Optimization of Disc Cutting Methods

When attempting to put full discs through the bone mill with bone grindings, the discs were unable to be ground by the bone mill. The annulus fibrosus (AF) section of the discs was too rigid and fibrous. Softer portions of the disc, such as the nucleus pulposus (NP), passed through the blade but remained stuck in the blade openings. This caused the AF to pile up in the mill housing. A weight is included in the bone mill kit, and it is necessary for forcing material into the milling blades. Placing this weight on top of the remaining AF portions caused the material to compact into a rigid and dry mass that was unable to be ground. No material was able to pass through the milling blades when this happened.

An idea to shred or cut the discs into smaller pieces was proposed. Several methods of cutting were tested on multiple donors' discs, including a frozen and thawed donor spine (W437522000582) and a fresh donor spine (W437523000266) (Table 4). Surgical scissors, a scalpel, and paper scissors were all tested but could not effectively cut through the AF without a great deal of force and time. A pizza cutter and an onion dicer were also tested. The pizza cutter was completely ineffective with all the discs, while the onion dicer was able to cut through very thin discs (1 to 2 mm thickness), but not larger discs (>3 mm thickness). The best option, which was incorporated into the process, was a pair of meat shearers. These effectively cut through all the disc material. A full disc could be cut in 30 seconds, with pieces approximately 0.5 to 1.0 cm in diameter.

TABLE 4
Disc Cutting Method Comparison

| Study | | Tested | Experimental | Experimental |
|---|---|---|---|---|
| # | Donor ID | Variables | Groups | Outcomes |
| 4 | W437522000582 | 1. Method | A. Surgical scissors | Stainless steel |
| | | of disc | B. Paper scissors | meat shears were |
| | | cutting | C. Scalpel | most efficient and |
| | | | D. Pizza cutter | effective to cut |
| | | | E. Onion dicer | through disc |
| | | | F. Meat shears | |
| 5 | W437523000266 | 1. Method | A. Surgical scissors | Stainless steel |
| | | of disc | B. Paper scissors | meat shears were |
| | | cutting | C. Scalpel | most efficient |
| | | | D. Pizza cutter | and effective to cut |
| | | | E. Onion dicer | through disc |
| | | | F. Meat shears | |

### Example 5: Disc-to-Bone Ratio Optimization

After the initial proof-of-concept testing, which demonstrated the ability of the bones and discs to be milled concurrently in the Fortios Bone Mill, several process parameters needed to be analyzed to determine what would create the most cohesive product.

The first process parameter to analyze was the amount of disc that would be mixed with the bone grindings. To test this, different ratios of disc material to bone grindings were proposed, based on what had been observed during the proof-of-concept testing. Three groups with different disc-to-bone ratios were tested: 1:9, 3:17, and 1:4, which correspond to a final product with 10% disc, 15% disc, and 20% disc, respectively. A fourth group, XXXF bone grindings with no disc, served as the control.

Initial conversations with the Development team concluded that a small grind size would be preferable for this product; therefore, the Fortios Bone Mill XXXF blade, which can grind material to sizes between 0.5 and 1 mm in diameter, was to be used in the process testing. The first iteration of the process started by mixing the discs (cut to 1 cm) with the previously milled XXF bone grindings using forceps, then this combined tissue was run through the bone mill at the same time and ground by the XXXF blade. Rinse Media was used to wash the material through the bone mill. Following the creation of each test group's product from the bone mill, each group was processed according to a similar procedure that is used to produce compositions described herein: they were all put into different sterile bowls with a 500 mL solution that consisted of 2.5% HSA, 10% dimethyl sulfoxide (DMSO), and Plasma-Lyte A. Each bowl was placed on a shaking incubator for 1 hour at a speed of 150 RPM. After shaking, the samples were decanted and underwent three washes in 10% DMSO in Plasma-Lyte A. For each wash, 125 mL of the 10% DMSO solution was poured into each bowl, the bowl was briefly shaken by hand, and then decanted. After the washes, each group was aliquoted into 2 to 3 15-mL cryovials with 6 to 8 g of sample each and cryopreserved in a −86° C. freezer.

In this initial testing, it was observed that the disc, regardless of concentration, continually remained lodged in between the XXXF mill blade and the mill housing due to its gelatinous and fibrous nature. It prevented material from passing through the milling blades and increased processing time by a large margin, compared to the control group. This was observed on every donor processed with this procedure: donors W437523000269, W437523000272, W437523000276, and W437523000278 (Table 5). Additionally, higher ratios of disc to bone yielded increased processing times (>60 minutes for a 100 g sample), with greater amounts of disc material clogging the milling blade. Different methods of unclogging the milling blade were attempted, including pouring Rinse Media into the mill, but a noticeable amount of disc material was still unable to be dislodged from between the blade and housing. This could pose many issues when the process is scaled up. One issue would be that concentration of disc material in the final product would be lower than the calculated amount due to the amount of disc material lost in the mill from clogging. Another issue for scaling up is that this method of processing would increase process times further when an entire donor is processed.

TABLE 5
Disc-to-Bone Ratio Testing

| Study # | Donor ID | Tested Variables | Experimental Groups | Experimental Outcomes |
|---|---|---|---|---|
| 6 | W437523000269 | 1. Disc-to-bone | A. XXXF Bone grindings | 15% and 20% disc products were |
| | | ratio | B. 10% Disc product | more cohesive than 10% disc |
| | | | C. 15% Disc product | product. All groups more |
| | | | D. 20% Disc product | cohesive than XXXF bone |
| | | | | grindings |
| 7 | W437523000272 | 1. Disc-to-bone | A. XXXF Bone grindings | 15% and 20% disc products were |
| | | ratio | B. 10% Disc product | more cohesive than 10% disc |
| | | | C. 15% Disc product | product. All groups more |
| | | | D. 20% Disc product | cohesive than XXXF bone |
| | | | | grindings |
| 8 | W437523000276 | 1. Disc-to-bone | A. XXXF Bone grindings | 15% and 20% disc products were |
| | | ratio | B. 10% Disc product | more cohesive than 10% disc |
| | | | C. 15% Disc product | product. All groups more |
| | | | D. 20% Disc product | cohesive than XXXF bone |
| | | | | grindings |
| 9 | W437523000278 | 1. Disc-to-bone | A. XXXF Bone grindings | 15% and 20% disc products were |
| | | ratio | B. 10% Disc product | \|more cohesive than 10% disc |
| | | | C. 15% Disc product | product. All groups more |
| | | | D. 20% Disc product | cohesive than XXXF bone |
| | | | | grindings |

Frozen samples from all four donors were thawed and placed through a series of cohesiveness tests. Observations for each group were collected and compared to the control group. These tests included a drop test from 1 meter, a bottle shake test, and a handling/moldability test. The drop test was quantified by mass loss, where the largest portion of product remaining is weighed and compared to the pre-drop mass. The shake test was analyzed by particulate count. The handling/moldability test was a subjective assessment to analyze the products' ability to hold together.

The control (XXXF bone grindings) samples for all 4 donors crumbled on impact after being dropped and particulates from the product were released everywhere in the bottle after being shaken vigorously. The product can stick together and contains moldable flexibility but still has a level of fragility. The overall texture is coarse.

The 10% disc group samples exhibited increased cohesiveness due to the addition of disc material. This group performed well in the drop test, losing only 0.1 grams of material on average likely due to moisture loss as there was not any product separation. However, the bottle shake test yielded a different result. This group did not undergo much particulate separation, but the product did split into multiple pieces on multiple sample shakes. The moldability of this group was better than the control, and product separation occurred when pulled apart with minimal force. There were a few flakes of bone grindings that came off while manipulating the product.

The 15% and 20% disc groups retained all their mass after being dropped and had no product separation during the shake tests. Upon observation, both could be molded more easily and did not have any flaking while handling the product. Both appeared to have a similar consistency and texture to dough, but the 20% group had a slimier texture. The 15% disc concentration was determined to be the best concentration to use because it was the lowest disc percentage that remained intact during the different tests.

### Example 6: Disc Cutting and Homogenization

Due to the clogging of the Fortios Bone Mill observed during processing with disc and bone grindings at the same time, it was decided that a new method for homogenizing the discs needed to be implemented.

The first processing alteration attempted was to grind the discs in the bone mill separate from the XXF bone grindings, in the hopes that isolating the materials would help prevent clogging. This method proved to be more effective in grinding the discs to smaller shreds and caused less clogging of the mill, which reduced processing times in comparison to the combined milling. However, the amount of disc being lost in the mill was still substantial, as observed by the remaining tissue on the bone mill blade. It also had variable results, with disc shredding processes taking anywhere from 30 minutes to an hour. Finally, the discs still came out in large shreds (>1.0 cm) at times and were not homogenous, which would affect the consistency and appearance of the final product. A new method for grinding the discs was needed, while the bone grindings could continue to be milled from XXF bone grindings into XXXF size grindings using the Fortios Bone Mill.

Alternative methods for pre-grinding the discs were proposed. Some ideas included different shredding devices, such as an herb grinder, a salt and pepper grinder, and a food processor. After searching for tissue grinding instruments that could be autoclaved, a laboratory grade homogenizer was purchased. The model obtained was the ProScientific Pro250 Homogenizer, along with its compatible 20 mm×200 mm Generator Probe. Homogenization proof-of-concept tests were started on discs from donor W437523000283 (Table 6). As instructed by the distributors of the homogenizer and probe, VWR, the homogenization should be conducted in liquid. In the initial tests full discs were first submerged in water within a 250 mL sterile jar. As this was proof-of-concept testing, there were no initial measured ratios of disc to media; water was added incrementally until an amount that would allow for full homogenization was used. The homogenizer struggled to shred a full disc at any of its speed settings. However, when cutting up the discs into pieces (~0.5 cm in diameter) with the meat shearers before putting them into water, the pieces of disc were homogenized by the ProScientific Homogenizer with minimal residual chunks of disc in suspension.

TABLE 6
Disc Homogenization Testing

| Study # | Donor ID | Tested Variables | Experimental Groups | Experimental Outcomes |
|---|---|---|---|---|
| 10 | W437523000283 | 1. ProScientific | A. Full discs | Cut discs were able to be |
| | | homogenization | B. Cut discs | homogenized with minimal |
| | | with whole and | | remaining disc pieces; whole |
| | | cut discs | | discs did not homogenize |
| 11 | W437523000286 | 1. Disc-to-liquid | A. Discs homogenized | Discs homogenized in Rinse |
| | | ratio | in Plasma-Lyte | Media created foaming whereas |
| | | 2. Plasma-Lyte vs. | B. Discs homogenized | there was none when done in |
| | | Rinse Media | in Rinse Media | Plasma-Lyte; disc to liquid |
| | | | C. Disc-to-liquid ratio of | ratio of 1:1 couldn't be fully |
| | | | 1:1 | homogenized whereas the 1:9 |
| | | | D. Disc-to-liquid ratio of | ratio group was able to be |
| | | | 1:9 | homogenized |
| 12 | W437523000288 | 1. Disc-to-liquid | A. Disc-to-liquid ratio of | Disc to liquid ratio of 1:1 |
| | | ratio | 1:1 | couldn't be fully homogenized |
| | | | B. Disc-to-liquid ratio of | whereas the 1:9 ratio group was |
| | | | 1:9 | able to be homogenized |

The disc and water mixture had a similar consistency to molasses with its viscous nature. Unlike the bone mill, the homogenizer created a smooth, uniform mixture of disc material. After grinding the XXF bone grindings to the XXXF size, the disc suspension was mixed with bone grindings, and the combined product was molded into a ball by hand. The resulting product held together without shedding bone grindings and showed remarkable cohesiveness. The product had a consistency similar to chewing gum and could be easily shaped and molded without any separation. The amount of disc suspension and bone grinding material was not recorded.

After this initial test, a ratio of disc to media by mass needed to be determined to be used for further testing of the homogenizer. Using discs from donors W437523000286 and W437523000288, several different concentrations of disc in Plasma-Lyte A were tried along with disc in Rinse Media. The disc and Rinse Media was abandoned after a couple attempts as the HSA created a significant amount of bubbling during homogenization. A ratio of 1:1 disc to Plasma-Lyte by mass was tried initially, and there was difficulty homogenizing the discs. A ratio of 1:9 disc to Plasma-Lyte by mass, or 10% disc by mass, was subsequently tried and settled upon, as it allowed for a more complete homogenization than some higher disc content groups, which had chunks of discs remaining.

### Example 7: Disc Region Testing

To determine what disc regions should be included to create the most cohesive final product, three donors were tested (W437523000288, W437523000296, and W437523000297) with three experimental groups each (along with a control of bone grindings with no disc) (Table 7). The three experimental groups consisted of products made with AF disc alone, NP disc alone, and whole discs. During preliminary testing, it was determined that cartilaginous endplate, fascia, fat, and muscle should be cut away from the NP and AF disc tissue, as these extraneous tissues would not increase the cohesiveness of the final product, and often caused noticeable debris in the final product when not removed during disc debriding. Each experimental group was processed by first debriding the discs, then cutting and separating the AF and NP from each other on some of the discs, and finally weighing out 15 g for each group of AF, NP, and whole disc. Each disc group was added to 135 mL of Plasma-Lyte A and homogenized using the ProScientific homogenizer. Then each group was added to 85 g of XXXF bone grindings. These were shaken to mix in a sterile bowl, and then all four groups went through a 1-hour Treatment Media shake with 125 mL of media added to each bowl (¼ of 500 mL each). Each group was decanted and then washed once with Plasma-Lyte and 10% DMSO. The final product was decanted, placed in vials, and frozen in a −86° C. freezer.

TABLE 7
Disc Region Testing

| Study # | Donor ID | Tested Variables | Experimental Groups | Experimental Outcomes |
|---|---|---|---|---|
| 12 (also | W437523000288 | 1. Disc regions | A. AF disc | Product with AF disc and with |
| used for disc | | used in the | B. NP disc | whole disc had similar |
| homogenization | | final product | C. Whole disc | cohesiveness, both were more |
| testing) | | | | cohesive than product with NP disc |
| 13 | W437523000296 | 1. Disc regions | A. AF disc | Product with AF disc and with |
| | | used in the | B. NP disc | whole disc had similar |
| | | final product | C. Whole disc | cohesiveness, both were more |
| | | | | cohesive than product with NP disc |
| 14 | W437523000297 | 1. Disc regions | A. AF disc | Product with AF disc and with |
| | | used in the | B. NP disc | whole disc had similar |
| | | final product | C. Whole disc | cohesiveness, both were more |
| | | | | cohesive than product with NP disc |
| 15 | W437523000303 | 1. Disc regions | A. AF disc | Product with whole disc had better |
| | | used in the | B. Whole disc | subjective handling and |
| | | final product | | cohesiveness than that with AF disc |
| | | | | according to surgeons and |
| | | | | distributors |
| 16 | W437523000305 | 1. Disc regions | A. AF disc | Product with whole disc had better |
| | | used in the | B. Whole disc | subjective handling and |
| | | final product | | cohesiveness than that with AF disc |
| | | | | according to surgeons and |
| | | | | distributors |
| 17 | W437523000313 | 1. Disc regions | A. AF disc | Product with whole disc had better |
| | | used in the | B. Whole disc | subjective handling and |
| | | final product | | cohesiveness than that with AF disc |
| | | | | according to surgeons and |
| | | | | distributors; confirmed the use of |
| | | | | whole disc in the final product |

Test Methods: An official test to objectively determine cohesiveness had not yet been developed. Therefore, subjective analysis was conducted on thawed samples from each group of each donor. Some of the subjective tactile analysis consisted of multiple lab personnel ripping thawed samples with gloved hands to see how easily the samples could be pulled apart. The samples were also formed into different shapes to see how easily they could be formed and applied to surgical sites. Finally, the samples would be dropped and even tossed downward onto the lab bench with a sterile drape, to see how easily they crumbled, or if any flakes of bone grindings came off the main sample.

Results: As seen in previous proof-of-concept testing, the control group of XXXF bone grindings performed the worst in subjective handling testing as compared to the three groups with discs. It could not hold shape during the tactile testing and separated completely when dropping the sample from any height. Compared to the AP disc alone and whole disc groups, thawed samples from the group made with NP disc alone had the next lowest ability to hold together during subjective handling analysis. It crumbled rapidly in the hands of the lab personnel and broke apart more easily during the drop testing, frequently splitting apart into several pieces. The laboratory personnel doing the subjective analysis could not find a noticeable difference between the AP disc alone samples and whole disc samples from any of the donors. Samples from both groups held their shape during the drop testing and the handling analysis.

External, Blinded Analysis: With the initial subjective analysis showing the decreased cohesiveness in the NP disc alone samples, additional samples for this group were not created for further testing. Three more donors (W437523000303, W437523000305, and W437523000313) were processed as described above, except with only control (XXXF bone grindings), AP disc alone, and whole disc samples created. These samples were labeled as “Demo” vials, with specific numbering for each sample type, and sent to multiple distributors and hospital systems. The samples were looked at by surgeons and distributors, who unanimously informed our team that the samples matching the numbers for our whole disc product had the best handling and cohesiveness properties. With this information, along with the additional justification that more whole disc could be used to create more product, whole discs would be used in all future process testing.

### Example 8: Centrifuge Liquid Removal

During the previous processes of six donors, it was observed while doing multiple decanting procedures with several groups that it takes a significant amount of time (>15 minutes for each decant), and a lot of manipulation with forceps, to decant liquid from the combined disc and bone grindings product. The discs have proteoglycans which are very hydrophilic and prevent the liquid from easily being removed. Product was frequently lost during the decanting process. To remove liquid from the product, a few methods were suggested, including a screw-press and lap sponges. Additional testing methods were attempted on donors W437523000315, W437523000321, and W437523000322 (Table 8). The lap sponges proved quite effective, but were inefficient, and could potentially flake material off into the product and leave product behind attached to the lap sponges. Finally, centrifuging the product and media in conical tubes was tested at an initial speed of 500×g for 5 minutes. Disc suspension was centrifuged alone and then combined bone grindings and disc suspension centrifugation was also tested. The disc suspension did not show any separation at this speed, while the combined bone and disc suspension did not show a full separation from the media. The process was repeated at 1000×g for 5 minutes with a significant amount of liquid separation noticed. While processing the final donor tested with the 1000×g centrifugation step, the amount of liquid removed after each centrifugation was weighed. The first decanting mass was within 10 g of the liquid added to create the disc suspension and the additional media added. The additional decanting steps were all within 10 g of the liquid added to the product. This indicated that the centrifuge steps were removing approximately the same amount of liquid that was added to the product for washes. Therefore, a centrifugation step was incorporated into the process after any media addition.

TABLE 8
Liquid Removal Testing

| Study # | Donor ID | Tested Variables | Experimental Groups | Experimental Outcomes |
|---|---|---|---|---|
| 18 | W437523000315 | 1. Methods for | A. Lap sponges | Both methods were ineffective: Lap |
| | | liquid removal | B. Manual pressing | sponges clung to bone grindings and left |
| | | | | behind residue and could not be used; |
| | | | | manual pressing had to be done in the |
| | | | | open and on absorbent wipe to remove |
| | | | | liquid |
| 19 | W437523000321 | 1. Methods for | A. Centrifugation | Centrifugation at 500× g was somewhat |
| | | liquid removal | at 500× g | effective; at 1000× g a significant |
| | | | B. Centrifugation | amount of liquid was removed from |
| | | | at 1000× g | product |
| 20 | W437523000322 | 1. Methods for | A. Centrifugation | Centrifugation for 5 minutes at |
| | | liquid removal | at 1000× g | 1000× g was effective and chosen as |
| | | | (verification) | the method for removing liquid from |
| | | | | the combine product; 250 mL conical |
| | | | | tubes were difficult to remove product |
| | | | | from, 500 mL conical tubes were |
| | | | | chosen over 250 mL conical tubes |

With these initial attempts at centrifugation of the product, 250 mL conical tubes were used. However, after decanting the liquid from the 250 ml conical tubes, it was time consuming to pull the pellet of product out bit-by-bit using forceps. The 250 ml conical tubes were also limited in the amount of total product and media they could hold in any given centrifugation. Therefore, 500 mL conical tubes with a wider opening were used for the centrifugation steps and showed similar results to centrifuging the product in 250 ml conical tubes, but with increased ease of access post-decanting.

Example 9: Disc-to-Bone Ratio Retesting with Homogenized Disc

Since the initial testing of disc-to-bone ratios was conducted using the Fortios Bone Mill instead of the ProScientific homogenizer, which produces different disc consistency, the disc-to-bone ratios were retested with the new disc homogenizing method. Donor W437523000301 was processed according to the established procedure at the time and divided into three different final product disc percentages: 10%, 15%, and 20%. Samples from these groups were frozen and later thawed. Using the handling and drop tests described above, it was seen that 15% and 20% disc product samples were more cohesive than the 10% disc product samples. The 20% disc sample had a slick feel to it, indicating it had more disc than was needed.

Three more donors (W437523000321, W437523000322, and W437523000326) were each processed with one control group (XXXF bone grindings alone) and two experimental groups: a 10% disc product and a 15% disc product (Table 9). The 20% disc product was abandoned, because it was already shown that the handling of 15% and 20% were similar, and it was reasoned that there was likely more disc in these products than in the bone mill products, where disc frequently got caught in the blade and did not end up in the final product. Therefore, each of these ProScientific homogenized samples should have more disc than their bone mill counterparts at the same calculated percentage of disc. It was reasoned that if 15% was already enough for the product processed in the bone mill, where some undetermined disc amount was lost (reducing the final disc percentage), it was possible that the 10% disc product could be the disc percentage needed when using the ProScientific homogenizer since all the disc is retained in the final product.

TABLE 9
Disc-to-Bone Ratio Re-Testing

| Study # | Donor ID | Tested Variables | Experimental Groups | Experimental Outcomes |
|---|---|---|---|---|
| 18 (also | W437523000315 | 1. Disc-to- | A. 10% Disc | 20% disc was re-affirmed to be more than |
| used for | | bone ratio | product | necessary; 15% disc product post-thaw |
| liquid | | | B. 15% Disc | was more cohesive than 10% disc product |
| removal | | | product | post-thaw; Confirmed validity of drop |
| testing) | | | C. 20% Disc | test by comparing post-thaw 15% disc |
| | | | product | product to three XXF bone grinding |
| | | | | product samples |
| 19 (also | W437523000321 | 1. Disc-to- | A. XXXF Bone | 15% disc product post-thaw was more |
| used for | | bone ratio | grindings | cohesive than 10% disc product post-thaw; |
| liquid | | | B. 10% Disc | both were more cohesive than XXXF |
| removal | | | product | grindings |
| testing) | | | C. 15% Disc | |
| | | | product | |
| 20 (also | W437523000322 | 1. Disc-to- | A. XXXF Bone | 15% disc product post-thaw was more |
| used for | | bone ratio | grindings | cohesive than 10% disc product post-thaw; |
| liquid | | | B. 10% Disc | both were more cohesive than XXXF |
| removal | | | product | grindings |
| testing) | | | C. 15% Disc | |
| | | | product | |
| 21 | W437523000326 | 1. Disc-to- | A. XXXF Bone | 15% disc product post-thaw was more |
| | | bone ratio | grindings | cohesive than 10% disc product post- |
| | | | B. 10% Disc | thaw; both were more cohesive than |
| | | | product | XXXF grindings; |
| | | | C. 15% Disc | Reaffirmed 15% disc was the best |
| | | | product | amount to use for the final product |
| 22 | W437523000330 | 1. Disc-to- | A. 15% Disc | Confirmed validity of drop test by |
| | | bone ratio | product | comparing post-thaw 15% disc product |
| | | confirmation | | to three XXF bone grinding product |
| | | | | samples |
| 23 | W437523000333 | 1. Disc-to- | A. 15% Disc | Confirmed validity of drop test by |
| | | bone ratio | product | comparing post-thaw 15% disc product |
| | | confirmation | | to three XXF bone grinding product |
| | | | | samples |

However, as with previous tests, the 15% disc product did better in the subjective handling and drop test than the 10% disc, while both performed better than the control. As before with the drop testing, the 15% disc product did not break apart in the drop test, but the 10% product split into multiple chunks. It has been concluded that 15% disc in the product was at or near a point of saturation with disc, and increased disc would not add to the cohesiveness, while decreased disc percentage created a product with reduced cohesiveness. All donors processed after donor W437523000326 were processed with 15% disc by mass.

Additional Data: A drop test was formalized after these donors had been tested. This test consisted of dropping the thawed sample in a ball from exactly 1 m onto a posterboard with a target to count the debris scattered and the distance the debris scattered from the main sample. There were not enough samples left from the previously processed groups to assess via this drop test, besides 15% disc samples from W437523000315. Two additional donors were processed with 15% disc from W437523000330 and W437523000333 and used for confirming the validity of a 1-m drop test. There were no pieces that broke off from any of these three samples during drop testing, in comparison to >25 pieces for every drop involving old samples of XXF bone grindings, as seen in Table 10. Finally, additional samples remained from the first disc-to-media ratio test (using the bone mill) with donor W437523000269; these samples were also used for the initial drop test verification. This donor had samples of XXXF bone grindings (control), and 10%, 15%, and 20% disc products. Three samples of bone grindings control scattered an average of 26.7 pieces of grindings to an average distance of 18.3 cm. Three samples of the 10% disc product scattered an average of 4.3 pieces of bone grindings over 17.5 cm (Table 11). The 15% and 20% disc product samples had 0 pieces of bone grindings break off the main sample when dropped. While this donor was not processed using the ProScientific homogenizer, it demonstrates similar outcomes were seen when performing the drop test on 15% and 10% disc product samples with discs fully homogenized by the ProScientific homogenizer.

TABLE 10
Drop Test Comparing XXF Bone Grindings with 15% Disc

| 3 Donors XXF Bone Grindings (No disc) | | | 3 Donors (15% Disc) | | |
|---|---|---|---|---|---|
| | Mean (n = 3) | Mean (n = 3) | | Mean (n = 3) | Mean (n = 3) |
| Donors | Furthest Scatter | Debris Count | Donors | Furthest Scatter | Debris Count |
| W-23-140 | 28.3 ± 2.9 | 75.7 ± 20.0 | W-23-315 | 0 ± 0 | 0 ± 0 |
| W-23-146 | 33.3 ± 2.9 | 45.3 ± 17.5 | W-23-330 | 0 ± 0 | 0 ± 0 |
| W-23-207 | 31.7 ± 2.9 | 53.3 ± 5.5 | W-23-333 | 0 ± 0 | 0 ± 0 |
| Average | 31.1 ± 2.6 * | 58.1 ± 15.8 ** | Average | 0 ± 0 | 0 ± 0 |

* indicates statistically significant difference between means with t-test (p < 0.001)
** indicates statistically significant difference between means with t-test (p < 0.005)

TABLE 11
Drop Test Comparing Four Disc-to-Bone Ratio Groups from One Donor
Donor W-23-269 Experimental Groups

| | | Mean (n = 3) | |
|---|---|---|---|
| | | Furthest | Mean (n = 3) |
| Donors | Group | Scatter (cm) | Debris Count |
| W-23-269 | XXXF Bone Grinding | 18.3 ± 2.9 | 26.7 ± 11.2 |
| | OssiGraft | | |
| W-23-269 | 10% Disc OssiGraft | 17.5 ± 13.9 | 4.3 ± 1.5 |
| | Prime Groups | | |
| W-23-269 | 15% Disc OssiGraft | 0 ± 0 | 0 ± 0 |
| | Prime Groups | | |
| W-23-269 | 20% Disc OssiGraft | 0 ± 0 | 0 ± 0 |
| | Prime Groups | | |

### Example 10: Bone Grinding Size

In some embodiments, the methods of making the compositions include a product with XXXF bone grindings, as the disc and bone grindings were going to be re-ground together after bone marrow had been eluted from the bone grindings. After switching to a process that grinds the discs separate from the bone grindings, a process that does not re-grind the bone grindings from XXF to XXXF bone grinding size was considered, to save on processing time and resources. Although it had already been established that XXXF bone grindings alone were more cohesive than XXF bone grindings alone, it was proposed that either grind size might have similar cohesiveness when combined with homogenized disc. Initially, two donors were processed using the bone mill with two groups: homogenized discs with XXF bone grindings and homogenized discs with XXXF bone grindings; an additional donor was processed with the same groups, but with discs homogenized using the ProScientific homogenizer.

During the initial phase of process development, when using the Fortios Bone Mill to both re-grind bone grindings and shred discs, cohesiveness testing was done for XXF bone grindings combined with disc (15% disc), and compared to XXXF bone grindings combined with disc (15% disc), and a control of XXF bone grindings without disc (Table 12). The cohesiveness testing was conducted on samples for two donors (W437523000276 and W437523000278). The control for both donors showed the lowest cohesiveness, falling apart while handling, and unable to be molded into shapes. The XXF bone grindings combined with homogenized disc samples demonstrated better cohesiveness than the control but had noticeably worse handling than the samples of XXXF bone grindings combined with homogenized disc. Bone grinding flakes came off readily when handling samples of the XXF grindings combined with disc, whereas the XXXF bone grindings combined with disc were able to be easily shaped and did not flake off any bone grindings.

TABLE 12
Bone Grinding Size Testing

| Study # | Donor ID | Tested Variables | Experimental Groups | Experimental Outcomes |
|---|---|---|---|---|
| 8 (also | W437523000276 | 1. XXF vs. | A. XXF bone grindings | XXXF bone grindings with disc in the |
| used for | | XXXF bone | B. XXF bone grindings | final product was more cohesive than |
| disc-to- | | grind size in | with disc | the other two groups |
| bone ratio | | final product | C. XXXF bone | |
| testing | | | grindings with disc | |
| 9 (also | W437523000278 | 1. XXF vs. | A. XXF bone grindings | XXXF bone grindings with disc in the |
| used for | | XXXF bone | B. XXF bone grindings | final product was more cohesive than |
| disc-to- | | grind size in | with disc | the other two groups |
| bone ratio | | final product | C. XXXF bone | |
| testing) | | | grindings with disc | |
| 25 | W437523000369 | 1. XXF vs. | A. XXF bone grindings | XXXF bone grindings with disc in the |
| | | XXXF bone | B. XXF bone grindings | final product was more cohesive than |
| | | grind size in | with disc | the other two groups; Confirmed the |
| | | final product | C. XXXF bone | use of XXXF bone grindings with |
| | | | grindings with disc | disc in the final product |

With the change in disc homogenization methods to the ProScientific, it was determined that a comparison of XXF bone grindings with homogenized discs to XXXF bone grindings with homogenized discs should be done with an additional donor, since the disc homogenization by the Fortios Bone Mill and the ProScientific homogenizer were different in consistency. Donor W437523000369 spine was processed with an XXF bone grindings control, an experimental group of XXF bone grindings combined with homogenized disc (15% disc), and an experimental group of XXXF bone grindings combined with homogenized disc (15% disc). Like the results with the bone mill tests described in the previous paragraph, the control had the worst cohesiveness. The samples from the XXXF bone grinding combined with disc had much greater cohesiveness than the XXF bone grindings with homogenized disc and the control samples. With these results, all donors processed for testing besides these three donors, were produced with bone grindings that had been reground to the XXXF size.

Additional Note about Grind Size: During HPC, Marrow process testing with different Fortios Bone Mill blades, the XXF blade was selected for creating a grind size that allowed the most marrow to elute from the bone. The XXXF blade creates smaller grind size; however, when using it to grind whole vertebral bodies down to bone grindings, it caused an increase in temperature that could affect cell viability. Additionally, it caused clogging of the bone marrow filter kit and created grind sizes so small that it affected the QC cell counting machine. For this reason, XXF bone grindings can have marrow eluted from them, and then can be reground to XXXF grindings, without the negative impacts that would affect the HPC, Marrow process. The temperature increase seen when grinding vertebral bodies to XXXF grindings is not observed when grinding XXF grindings to XXXF grindings. Furthermore, there is no concern for clogging the bone marrow filtration kit or causing issues with HPC, Marrow cell counting, as the marrow was eluted prior to the regrinding step. Therefore, additional validation of the use of the XXXF blade is not necessary, as none of the initial concerns for use of it with HPC, marrow apply.

### Example 11: Alternative Disc Homogenization Methods

Rationale for New Method: Using the ProScientific homogenizer created more consistently homogenized discs than the Fortios bone mill; however, the ProScientific homogenizer was very user dependent. It entailed maneuvering the homogenizer generator head through a jar filled with disc and media, which often lead to inconsistent homogenization. There were often a few large pieces that never were homogenized with each donor processed. For instance, large pieces of disc were observed in the final product during the first full process, when using a process that resembled a planned final process, with donor W437523000354 (Table 13). Another downside to the ProScientific homogenizer is that the discs had to be pre-cut with meat shears before homogenization, which would expose them to extra handling from Production personnel. With all these considerations, multiple other attachments and machines were demoed to find an alternative device that could homogenize whole discs and do it consistently in a closed system.

TABLE 13
Alternate Disc Homogenization Testing

| Study # | Donor ID | Tested Variables | Experimental Groups | Experimental Outcomes |
|---|---|---|---|---|
| 24 | W437523000354 | 1. Full process | A. Homogenization via | Confirmed an entire process |
| | | using | ProScientific | could be completed with discs |
| | | ProScientific for | homogenizer | homogenized by the |
| | | homogenization | | ProScientific homogenizer |
| 26 | W437523000388 | 1. Homogenization | A. Homogenization with | Confirmed use of |
| | | with Waring | 10% disc and 90% | homogenization with Waring |
| | | blender | Plasma-Lyte | blender produced consistent |
| | | | B. Homogenization with | homogenized discs in a closed |
| | | | 20% disc and 80% | container at multiple ratios of |
| | | | Plasma-Lyte | disc to Plasma-Lyte |
| 27 | W437523000396 | 1. Full process | A. Homogenization of | Confirmed an entire process |
| | | using Waring | whole discs with | could be completed with discs |
| | | blender for | Waring blender | homogenized by the Waring |
| | | homogenization | | lab blender, with final product |
| | | | | similar to previous processes |
| | | | | using the ProScientific |
| 29 | W437523000426 | 1. Homogenization | A. Homogenization of | Whole discs were unable to be |
| | | with Silverson | whole discs with | homogenized by the Silverson |
| | | Lab mixer | Silverson lab mixer | lab mixer even at multiple |
| | | | | speeds and volumes |

ProScientific: The first alternative was another product from ProScientific called a Safety Sealed Chamber Assembly with Blade Assembly. The homogenizer motor from ProScientific could be attached to this chamber assembly instead of the previously used generator probe. The container had an interior spinning blade attached to a rotating rod that came out of the lid of the closed container and attached to the motor. This device proved entirely ineffective at homogenizing whole or cut discs at any of the speeds attempted and with multiple donor's discs

Silverson: Another alternative was a Silverson Model LS4U Sealed Laboratory Mixer with a 1 L glass vessel and slotted disintegrating head. Discs from Donor W437523000426 were used to test the Silverson machine during the demo. While this model was capable of homogenizing pre-cut discs, it was incapable of cutting full discs at any speed setting or media volume and was not used for further testing.

Waring: The third alternative for homogenization was the Waring 3 Speed 4-Liter Blender with a 1 L adaptor and 1 L blending container. The blender was able to homogenize whole discs in a closed container without any manipulation. The first trial with discs from Donor W437523000388 showed consistent homogenization at 20% and 10% disc-to-media concentrations in the blending container. This blender with a 1 L blending container was used for all subsequent donors. Any process parameter testing done while homogenizing the discs with the ProScientific homogenizer (Disc-to-Bone Ratio; Disc Region; Bone Grindings Size; and Centrifuge Liquid Removal) was not repeated with the Waring blender, as the machines produced similar consistency of disc suspension, and it was concluded that any testing with the Waring blender would create similar results to the testing already done while using the ProScientific homogenizer. This was verified when performing the entire process, using Donor W437523000396 and homogenizing discs with the Waring blender; the final post-thaw samples had similar consistency and cohesiveness to previous samples made using the ProScientific homogenizer.

Waring Blender w/Eberbach Blending Container: The 1 L blending container that was provided by Waring's distributor was not secure and would leak material out the top. As Waring had discontinued manufacture of their screw-top 1 L stainless steel blending container, an alternative custom product made by Eberbach company, that was also a 1 L screw-top all stainless-steel blending container, was purchased. This blending container could be used on the Waring blender already in-house. The amount of time needed for consistent blending and the high temperatures produced during blending, when using the Waring blender, were tested and discussed further below.

### Example 12: Disc Decontamination

To reduce the likelihood of final product microbial contamination, multiple common decontaminants were tested on the intervertebral discs. An added decontamination step for the discs was considered to be consistent with the vertebral bodies, which go through a bleach wash prior to grinding.

An initial proof-of-concept run was conducted by running a full process using donor W437523000415, in which one half of the discs were decontaminated in bleach (0.52% sodium hypochlorite) for 15 minutes on a shaker before being homogenized, and the other half of the discs were homogenized with no decontamination to serve as the control (Table 14). It was observed that bleach caused some disintegration and discoloration of the disc tissue during processing. The final post-thaw product with bleach as the decontaminant was similar to the control product in regard to subjective cohesiveness and handling. The discoloration and flaking of the disc during bleach decontamination prompted testing other decontaminants.

TABLE 14
Disc Decontamination Testing

| Study # | Donor ID | Tested Variables | Experimental Groups | Experimental Outcomes |
|---|---|---|---|---|
| 28 | W437523000415 | 1. Disc | A. 0.52% Sodium | There was no observable |
| | | decontamination | Hypochlorite (Bleach) | difference between post-thaw |
| | | | B. No decontamination | samples from each group; |
| | | | (Control) | bleach caused discoloration |
| | | | | and disintegration of the whole |
| | | | | discs during decontamination |
| 30 | W437523000441 | 1. Disc | A. 0.52% Sodium | Drop test particle count: |
| | | decontamination | Hypochlorite (Bleach) | IPA = Control = Bleach > H₂O₂; |
| | | | B. 3% Hydrogen Peroxide | Shake test particle count: |
| | | | C. 70% IPA | Bleach > H₂O₂ > IPA = Control |
| | | | D. No decontamination | |
| | | | (Control) | |
| 31 | W437523000447 | 1. Disc | A. 0.52% Sodium | Drop test particle count: |
| | | decontamination | Hypochlorite (Bleach) | H₂O₂ > IPA = Bleach > Control; |
| | | | B. 3% Hydrogen Peroxide | Shake test particle count: |
| | | | C. 70% IPA | Bleach > H₂O₂ > IPA > Control |
| | | | D. No decontamination | |
| | | | (Control) | |
| 32 | W437523000449 | 1. Disc | A. 0.52% Sodium | Drop test particle count: |
| | | decontamination | Hypochlorite (Bleach) | H₂O₂ > IPA = Control = Bleach; |
| | | | B. 3% Hydrogen Per\|oxide | Shake test particle count: |
| | | | C. 70% IPA | H₂O₂ > Bleach > IPA > Control |
| | | | D. No decontamination | |
| | | | (Control) | |
| 33 | W437523000465 | 1. Disc | A. 0.52% Sodium | Drop test particle count: |
| | | decontamination | Hypochlorite (Bleach) | H₂O₂ > Bleach > Control > IPA; |
| | | | B. 3% Hydrogen Peroxide | Shake test particle count: |
| | | | C. 70% IPA | IPA = Control > H₂O₂ > Bleach |
| | | | D. No decontamination | |
| | | | (Control) | |
| 34 | W437523000476 | 1. Disc | A. 0.52% Sodium | Drop test particle count: |
| | | decontamination | Hypochlorite (Bleach) | Bleach = H₂O₂ > IPA > Control; |
| | | | B. 3% Hydrogen Peroxide | Shake test particle count: |
| | | | C. 70% IPA | Bleach > H₂O₂ = Control > IPA; |
| | | | D. No decontamination | IPA and Control had lower |
| | | | (Control) | particle counts for both tests |
| | | | | than H₂O₂ and Bleach on |
| | | | | average, but with no |
| | | | | statistical significance for any |
| | | | | group difference. With the |
| | | | | potential damage (and |
| | | | | visually observed damage) |
| | | | | caused by H₂O₂ and bleach to |
| | | | | the discs, these methods were |
| | | | | ruled out for |
| | | | | decontamination. |

Five donors were processed (W437523000441, W437523000447, W437523000449, W437523000465, and W437523000476) with three experimental groups and one control group for each donor. The donors were all processed according to the established process at the time, except for the experimental group variables. The whole discs were divided into four groups with equal disc mass. The experimental groups consisted of discs placed on the shaker for 15 minutes in 200 mL of either 3% hydrogen peroxide (H2O2), 70% isopropyl alcohol (IPA), or 0.52% sodium hypochlorite (Bleach). The control group discs were not soaked in anything. After the 15-minute shake, each experimental groups' decontaminant was decanted. The discs were then soaked in 200 mL of Plasma-Lyte, shaken for 30 seconds, and then decanted again. This Plasma-Lyte wash and decant was repeated two more times for each group. The 0.52% sodium hypochlorite (Bleach) group also had hydrogen peroxide added for 30 seconds after the first Plasma-Lyte wash, to neutralize the hypochlorite. After the final decanting step, the discs were homogenized in Plasma-Lyte via the Waring lab blender, and then processed as usual. All the samples from these five donors were then placed in a −86° C. freezer. Several days after freezing, the samples were thawed and a drop test and shake test were conducted for each sample.

Test Methods: The drop test conducted was the same as that described in other examples disclosed herein. The shake test was newly implemented for these samples and consisted of shaking a 5 cc sample, rolled into a ball, in a 1 L sterile jar for 15 seconds. After shaking, the number of pieces that had separated from the main sample ball were counted. Pieces were determined as separate chunks of material; this did not include individual bone grinding flakes.

Results: The IPA and Control groups produced the lowest number of pieces/debris in both the shake test and the drop test. For the drop test, the average sample produced 0.60 pieces for the IPA group, 0.67 pieces for the Control group, 1.13 pieces for the Bleach group, and 1.53 pieces for the Hydrogen Peroxide group, as shown in Table 15. For the shake test, on average, the Control group produced 6.2 pieces, the IPA group produced 6.6 pieces, the Hydrogen Peroxide group produced 9.6 pieces, and the Bleach group produced 11.0 pieces (Table 16). However, none of these differences in means were statistically significant. Even without statistically significant differences, the IPA group was selected over the other two experimental groups because it had the lowest average amount of debris, and because it did not cause any noticeable disintegration of disc tissue, while the H2O2 and Bleach groups did. Also, during testing, it was observed that the drop test is less effective to distinguish differences within samples of the compositions described herein, than it is to distinguish differences between the present compositions and compositions known in the art. This is because most samples did not break or only had one piece of debris break off the main sample. However, even with just the data from the shake tests, the IPA group is still the most comparable to the control. Therefore, IPA was chosen as the decontaminant for the discs.

TABLE 15
Drop Test Comparing Disc Decontamination Methods

| Bleach Discs | | | Control Discs | | |
|---|---|---|---|---|---|
| | Mean (n = 3) | | | Mean (n = 3) | |
| | Furthest | Mean (n = 3) | | Furthest | Mean (n = 3) |
| Donors | Scatter (cm) | Debris Count | Donors | Scatter (cm) | Debris Count |
| W-23-441 | 6.7 ± 11.5 | 0.67 ± 1.15 | W-23-441 | 6.7 ± 5.8 | 0.67 ± 0.58 |
| W-23-447 | 3.3 ± 3.5 | 0.67 ± 0.58 | W-23-447 | 5.0 ± 8.7 | 0.33 ± 0.58 |
| W-23-449 | 5.0 ± 8.7 | 0.33 ± 0.58 | W-23-449 | 1.7 ± 2.9 | 0.33 ± 0.58 |
| W-23-465 | 15.0 ± 8.7 | 2.33 ± 0.58 | W-23-465 | 10.8 ± 3.8 | 1.67 ± 0.58 |
| W-23-476 | 18.3 ± 10.4 | 1.66 ± 1.15 | W-23-476 | 10.0 ± 17.3 | 0.33 ± 0.58 |
| Average | 9.7 ± 6.6 | 1.13 ± 0.83 | Average | 6.8 ± 3.73 | 0.67 ± 0.58 |
| IPA Discs | | | H₂O₂ Discs | | |
| | Mean (n = 3) | | | Mean (n = 3) | |
| | Furthest | Mean (n = 3) | | Furthest | Mean (n = 3) |
| Donors | Scatter (cm) | Debris Count | Donors | Scatter (cm) | Debris Count |
| W-23-441 | 11.7 ± 12.6 | 0.66 ± 0.58 | W-23-441 | 0.0 ± 0.0 | 0.00 ± 0.00 |
| W-23-447 | 6.7 ± 7.6 | 0.66 ± 0.58 | W-23-447 | 3.3 ± 5.8 | 1.00 ± 1.73 |
| W-23-449 | 3.3 ± 5.8 | 0.33 ± 0.58 | W-23-449 | 13.3 ± 7.6 | 1.67 ± 1.15 |
| W-23-465 | 2.5 ± 4.3 | 0.33 ± 0.58 | W-23-465 | 12.5 ± 4.3 | 3.33 ± 1.53 |
| W-23-476 | 7.5 ± 7.5 | 1.00 ± 1.00 | W-23-476 | 10.8 ± 7.2 | 1.67 ± 0.58 |
| Average | 6.3 ± 3.68 | 0.60 ± 0.28 | Average | 8.0 ± 5.97 | 1.53 ± 1.21 |

Note:
No statistically significant difference between Debris Count means of Furthest Scatter means of any groups based on ANOVA and Tukey HSD tests (significant p-value is <0.05)

TABLE 16
Shake Test on 5 Post-Thaw Samples Comparing Disc Decontamination Methods

| | Control Group | IPA Group | H₂O₂ Group | Bleach Group |
|---|---|---|---|---|
| Donors | Debris Count | Debris Count | Debris Count | Debris Count |
| W437523000441 | 3 | 3 | 6 | 9 |
| W437523000447 | 1 | 2 | 3 | 10 |
| W437523000449 | 1 | 3 | 15 | 8 |
| W437523000465 | 16 | 16 | 14 | 11 |
| W437523000476 | 10 | 9 | 10 | 17 |
| Average | 6.2 ± 6.6 | 6.6 ± 5.9 | 9.6 ± 5.1 | 11.0 ± 3.5 |

Note:
No statistically significant difference between Debris Count means of any groups based on ANOVA and Tukey HSD tests (significant p-value is <0.05)

Additional Data: To ensure that these results still applied when the process was scaled up for each group, three more donors (W437523000482, W437523000483, and W437523000484) were processed, with the discs divided in half to create an IPA experimental group and a non-decontaminated Control group. The IPA group and Control group were treated with the same methodology as described in other examples disclosed herein. With the consideration that the drop test was not effective at making distinctions between sample types, only the shake test was conducted. The IPA group split into 3.0 pieces on average, while the Control group split into 4.3 pieces on average (Table 17). This meant that samples from the IPA group were at least comparable, if not more cohesive than those from the Control group. All future donors processed after W437523000484 were processed with discs that were decontaminated with 70% IPA.

TABLE 17
Shake Test on Post-Thaw Samples Comparing
IPA Disc Decontamination to Control

| | Control Group | IPA Group |
|---|---|---|
| Donors | Debris Count | Debris Count |
| W437523000482 | 7.5 | 5.5 |
| W437523000483 | 3.0 | 2.5 |
| W437523000484 | 2.5 | 1.0 |
| Average | 4.3 ± 2.8 | 3 ± 2.3 |

Note:
No statistically significant difference between Debris Count means based on t-test (significant p-value is <0.05)

### Example 13: Disc-to-Media Ratio During Disc Homogenization

From the beginning of utilizing the Waring lab blender to homogenize disc, the discs were homogenized with Plasma-Lyte at a 1:4 ratio of disc to Plasma-Lyte, with 20% of the homogenized disc suspension mass consisting of disc material. However, during the disc decontamination testing (dividing the discs into four groups), to submerge the blending blades, the volume of Plasma-Lyte had to be increased. For the five donors, this resulted in a ratio of either 1:9 or 1:19 with 10% or 5%, respectively, of the disc suspension mass consisting of disc mass. While the IPA and Control groups were more cohesive than the Hydrogen Peroxide and Bleach groups, they were still less cohesive compared to previous donors processed. While these groups shed a couple flakes of bone grindings with the drop test or disintegrated with the shake test, previous donors held up completely with no debris in either test, although there is no formal data to verify this-just observational information. One of the major process differences between the decontamination groups and previous tests was that the discs were being ground with more liquid, which could affect the fibers or the disc retention in the final product upon centrifugation. Therefore, it was suggested to maintain a disc to Plasma-Lyte ratio in the disc suspension at 1:4 for all future runs.

No further official testing of the 1:4 disc to Plasma-Lyte ratio was conducted; however, the Eberbach 1 L stainless steel lab blending container has a recommended optimal volume maximum of 500 mL. It was observed that discs homogenized in any volume below 200 mL would often contain disc material with diameters greater than 0.5 cm due to the blades of the blending container not being completely submerged. With this range of 200 mL to 500 mL of material (assuming 1:1 mass: volume), the range of discs that can be added with a 1:4 ratio of disc to Plasma-Lyte is 40 g to 100 g. Most donors have a disc mass within this range, which means the 1:4 ratio will yield optimal use of the blending container. All donors processed after W437523000484 had discs that were homogenized with a 1:4 ratio of disc to Plasma-Lyte by mass.

With the implementation of disc decontamination with 70% IPA, it was observed that the discs often soaked up Plasma-Lyte in the post-IPA Plasma-Lyte washes. This additional Plasma-Lyte caused the post-decontamination disc mass to be higher than it was when weighed before the decontamination steps. Therefore, the starting disc mass prior to decontamination is to be used to calculate the total mass necessary to get 20% disc by mass. To provide an example to clarify this point, see the following breakdown.

### Equation:

(a) [Original mass of debrided discs]*5=[Total mass of disc and Plasma-Lyte]
(b) [Total weight of disc and Plasma-Lyte]−[Original mass of debrided discs+residual Plasma-Lyte mass post-decontamination]=[Total mass of Plasma-Lyte to add]

### Example

(a) There were 50 g of disc debrided from a donor.
(b) [50 g of original debrided disc]*5=[250 g of disc and Plasma-Lyte in final suspension]
(c) After decontamination, the discs retained some Plasma-Lyte, and the mass of disc and residual Plasma-Lyte was 80 g total.
(d) [250 g of disc and Plasma-Lyte in final suspension]−[80 g original mass of debrided discs+residual Plasma-Lyte mass post-decontamination]=[170 g Total mass of Plasma-Lyte to add]
(e) 170 g of Plasma-Lyte would be added to the discs and residual Plasma-Lyte prior to homogenization

All donors used for disc decontamination testing, along with all donors processed through Final Development process runs, used this method to calculate the media added to the discs for homogenization.

### Example 14: Disc Temperature Testing

From the beginning of disc homogenization testing, it was noted the homogenization caused the disc suspension temperature to rise to levels (30 to 55° C.) that could negatively impact bone cell viability when adding the disc suspension to the bone grindings. These elevated temperatures were observed with both the ProScientific homogenizer and the Waring lab blender. For early-stage processing, in order to cool the disc suspension down prior to adding it to the bone grindings, the disc suspension was added to a 1 L sterile jar and placed in the fridge until the temperature was at or below 25° C. Cooling the disc suspension in the fridge was not feasible for clinical processes, so alternative options were proposed. Initially, donor W437523000507 discs were used to test how long it would take for the discs to cool at ambient temperature, which took over 65 minutes (Table 18). After this initial testing, multiple solutions were tested, including the use of pre-cooled Plasma-Lyte, placing the suspension on a shaker, and using two different cooling containers that could be wiped with disinfectant and brought into the cleanroom. Discs from donors W437523000511, W437523000514, W437523000503, W437523000526, W437523000527, W437523000532, and W437523000534 were used in the testing. During testing, the cooling containers were removed from the −20° C. freezer just prior to use, while the disc suspension was placed in 1 L sterile jars and these jars were placed in the cooling containers. For some donors, pre-cooled Plasma-Lyte (5° C.) was added to the discs just prior to homogenization. After homogenization and placing the disc suspension in a new jar for all groups, the temperature of disc suspension in the jar was checked at intervals until the disc suspension was at or below 25° C. After the disc suspension cools to ≤25° C., the disc suspension would be added to the completed re-ground bone grindings. Pre-cooled Plasma-Lyte was rejected as an option because it was logistically problematic to pre-filter Plasma-Lyte, take it to a fridge outside the cleanroom, and then bring it back into the cleanroom for disc homogenization. Ultimately, a wine cooling sleeve was selected for the cooling method, as it lowered the temperature of the disc suspension at a faster rate than other cooling containers. However, due to supplier concerns, two flexible medical gel ice packs wrapped around the jar holding the disc suspension would be used for cooling instead.

TABLE 18
Disc Suspension Cooling Testing

| Study # | Donor ID | Tested Variables | Experimental Groups | Experimental Outcomes |
|---|---|---|---|---|
| 40 | W437523000507 | 1. Disc cooling | A. Ambient temperature | 330 g of disc suspension cooled to ≤25° |
| | | methods | cooling | C. after 65 minutes |
| 41 | W437523000511 | 1. Disc cooling | A. Pre-cooled Plasma-Lyte | 368 g of disc suspension at 1:9 disc to |
| | | methods | (5° C.) used during disc | Plasma-Lyte ratio cooled ≤25° C. after |
| | | | homogenization | 45 minutes |
| 42 | W437523000514 | 1. Disc cooling | A. Pre-cooled Plasma-Lyte | 234.5 g of disc suspension at 1:4 disc to |
| | | methods | (5° C.) used during disc | Plasma-Lyte ratio cooled ≤25° C. after |
| | | | homogenization | 45 minutes |
| 43 | W437523000503 | 1. Disc cooling | A. Pre-cooled Plasma-Lyte | 467.5 g of disc suspension at 1:4 disc to |
| | | methods | (5° C.) used during disc | Plasma-Lyte ratio, on the shaker for 60 |
| | | | homogenization; disc | minutes after homogenization, had not |
| | | | suspension placed on | cooled ≤25° C. after 120 minutes |
| | | | shaker | |
| 44 | W437523000526 | 1. Disc cooling | A. Pre-cooled Plasma-Lyte | 200 g of disc suspension for each group |
| | W437523000527 | methods | (5° C.) used during disc | at a 1:4 ratio of disc to Plasma-Lyte. |
| | W437523000534 | | homogeni\|zation | Wine cooling gel cooled the fastest of |
| | (Discs from | | B. Wine cooling sleeve | the three groups reaching ≤25° C. after |
| | donors combined) | | over jar with pre-cooled | 20 minutes. |
| | | | Plasma-Lyte (5° C.) | |
| | | | C. Wine cooling vacuum- | |
| | | | sealed container around | |
| | | | jar with pre-cooled | |
| | | | Plasma-Lyte (5° C.) | |
| 45 | W437523000532 | 1. Disc cooling | A. Wine cooling sleeve | 222.5 g of disc suspension at 1:4 disc to |
| | | methods | over jar with ambient | Plasma-Lyte ratio cooled ≤25° C. after |
| | | | temperature Plasma- | 40 minutes. Ambient temperature |
| | | | Lyte | Plasma-Lyte to be used to avoid the |
| | | | | need to refrigerate Plasma-Lyte. Wine |
| | | | | cooling sleeve initially chosen as |
| | | | | method for cooling disc suspension. |
| | | | | Replaced by similar concept ice gel |
| | | | | packs for quality reasons. |

A significant amount of the disc suspension was left behind in the 1 L sterile jars used for cooling due to the narrow bottleneck, so the container to hold the cooling disc suspension was switched to a 1.25 L stainless steel jar. This jar has an opening as wide as the container base and allows for disc suspension to be scraped out with a spatula more easily. The stainless steel also allows for more heat transfer from the disc suspension to the cooling sleeve. All Development donors processed after W437523000544 used the wine cooling sleeve to cool the disc suspension within a stainless-steel jar.

Disc Cooling Time: There is no set time limit as a point of control for how long the disc suspension can sit waiting (post-homogenization) until the next step of disc processing. The justification for having no time limit is that the cells and growth factors present in the disc suspension are not of value for the overall function of the graft. Therefore, viability and functionality of the cells and growth factors in the disc suspension, which may decrease the longer it sits, is of no concern for this process. The cells and growth factors present in the bone grindings are considered the source of cells and growth factors found in the final product. The homogenized disc is solely present in the graft to increase the cohesiveness and moldability of the product. The time the discs sit before homogenization and the time the suspension sits after homogenization have no impact on the cohesiveness and moldability of the product. Several tested donors had discs waiting for up to 5 hours prior to being used and the disc suspension frequently sat for up to an additional 4 hours prior to being combined with bone grindings in any given Development-stage process. These increased processing times and delays never appeared to have any influence on the ability of the homogenized discs to yield a cohesive and moldable final bone allograft product. Additionally, these delays in Development-stage processing runs go well beyond the amount of time discs or disc suspension would sit at ambient temperature while waiting to be processed for clinical production.

### Example 15: Mixing Prior to Packaging

During processing, after centrifugation of the final combined disc and bone grindings and decanting off the supernatant, it was observed that the resulting tissue needed to be mixed and recombined a final time to create a homogenous final product. This would ensure the disc and bone grinding are uniformly distributed throughout the product. During centrifugation, the disc material settles on the top of the tissue material within the conical tubes, while hard packed bone grindings form clumps at the bottom of the conical tubes. Several ideas were proposed for mixing the final combined tissue. Ultimately, the most feasible option was to use a standing over-head lab stirrer with an attachable mixing head to mix the material. The product material is stirred in a container that is manipulated by the operator to ensure full mixing. The selected mixer is a Heavy Duty Stirrer from INDCO, which has the high torque necessary to mix the material well.

Several prototypes of attachment heads were created to see which design would most effectively recombine the tissue without flinging the tissue around in the mixing container. More than a dozen mixing heads were trialed. The mixing heads were subjectively analyzed by three Development personnel based on their ability to remove bone grinding clumps and create a visually consistent final product. Originally, a mixing head designed to shred pulled pork was selected; however, the stainless-steel head was scraping the glass jar mixing container. Therefore, the team trialed multiple plastic-coated mixing heads and settled on a 4-blade mixing attachment from INDCO with a stainless-steel core covered in Teflon.

Originally, a 2-qt glass jar was selected as the mixing container because glass is non-immunogenic and if small debris ended up in the product, it would not be as much of a safety concern as if metal or plastic ended up in the final product. The glass jar was also transparent, which allowed the user to see the tissue more easily as it was being mixed. However, during a cleanroom processing run, the glass jar broke during mixing, so the team considered other options. Ultimately, a disposable plastic bowl (which is already used elsewhere in the process) was selected to hold the tissue while mixing. Mixing in the sterile plastic bowl showed similar results to mixing in the glass jar.

Several mixing speeds were tested with tissue from the same donor. 1000 RPM effectively mixed the tissue in under 3 minutes without flinging material out of the mixing container. As this speed mixed tissue consistently across multiple donors, it was selected as the speed for mixing.

Since the Heavy Duty Stirrer cannot be autoclaved, it is wiped with 70% IPA before placing into the BSC. Therefore, an ophthalmic clear plastic surgical drape was identified as it has an opening for the stirrer attachment and the rest of the drape covers the stirrer, preventing exposed portions of the stirrer from resting over the tissue during mixing.

### Example 16: Bone Re-Grind Media Volume

For all the donors processed before donor W437523000495, 500 mL of Rinse Media was used to wash the bone grindings through the bone mill during the re-grind from XXF size to XXXF size bone grindings. However, to be in line with AATB guidelines on volumes used in tissue processing, the volume of Rinse Media used for the wash was changed to be a 1:1 ratio of bone grindings mass to Rinse Media mass. This process change had no noticeable impact on the ability of the operator to effectively wash all the bone grinding material through the bone mill inlet. All donors processed after W437523000495 used the 1:1 ratio of bone grinding mass to Rinse Media mass when washing the bone grindings through the bone mill.

### Example 17: VBX Mass to Media Wash Ratio (Final Development Runs—Process 1)

During development testing, after the disc suspension and the bone grindings were combined, they were shaken in Treatment Media with 10% DMSO for 1 hour, and then decanted and washed three times. Each wash consisted of adding 500 mL of Plasma-Lyte with 10% DMSO, placing the combined tissue and media on the shaker at 150 RPM for 1 minute, centrifuging at 1000×g for 5 minutes, and then decanting.

By the time the first set (“Process 1”) of Final Development runs (donors W437523000544, W437523000546, and W437523000558) were started, the procedure was changed to leave DMSO out of the 1-hr shake with Treatment Media in order to prevent the cells from spending extra time in DMSO, in line with changes to the production process (Table 19). The time spent on the shaker was also increased to 5 minutes for the first and third washes, and 30 minutes for the second wash. This was altered to give the antimicrobials from the Treatment Media more time to elute out of the bone grindings, so that residual antimicrobials would not affect the external B/F (Bacteriostasis/Fungistasis) method validation testing. For the first two donors, W43752300544 and W437523000546, a 1:1 tissue mass to media mass ratio was used for the post-Treatment Media washes, to be in line with the most up-to-date iteration of the production process. When processing donor W437523000558, the volume of 10% DMSO in Plasma-Lyte used for the post-Treatment Media washes was increased to allow for an even greater dilution; the ratio of tissue mass to media mass added for the washes was 1:4. This increase was due to a measurement that determined additional media should be added to successfully dilute the Treatment Media enough to match the dilution done by the five washes performed during production. These plans and calculations were changed as described in the “Process 2 Alterations” section below.

TABLE 19
Final Development Run (Process 1) Testing

| Study # | Donor ID | Process Changes | Testing Performed | Experimental Outcomes |
|---|---|---|---|---|
| 46 | W437523000544 | 1. Minor process | A. Primary digest | Confirmed presence of viable cells; |
| | | alterations | viability | conditionally confirmed non- |
| | | 2. Washes: 1:1 solid | B. MLR | immunogenicity with the need to re- |
| | | mass to media ratio | C. Growth | test: confirmed presence of target |
| | | | factors | growth factors |
| 47 | W437523000546 | 1. Minor process | A. Primary digest | Confirmed presence of viable cells; |
| | | alterations | viability | conditionally confirmed non- |
| | | 2. Washes: 1:1 solid | B. MLR | immunogenicity with the need to re- |
| | | mass to media ratio | C. Growth | test: confirmed presence of target |
| | | | factors | growth factors |
| 48 | W437523000558 | 1. Minor process | A. Primary digest | Confirmed presence of viable cells; |
| | | alterations | viability | conditionally confirmed non- |
| | | 2. Washes: 1:4 solid | B. MLR | immunogenicity with the need to re- |
| | | mass to media ratio | C. Growth | test: confirmed presence of target |
| | | | factors | growth factors; external testing lab |
| | | | D. B/F testing | confirmed passing B/F growth |
| | | | | testing |

### Example 18: Process 2 Alterations (Final Development Runs—Process 2)

The process alteration that was considered and implemented after the first three Final Development runs (Process 1), was to wait until after the final wash of the bone grindings to add the disc suspension. There were two main reasons for implementing this change. The first is that external B/F testing method validation testing had failed several times due to too much residual antimicrobials in the final product. The residual antimicrobials can prevent microbes from growing and give a false negative, when a test sample may otherwise have positive sterility results. To reduce the likelihood of failing B/F testing, it was thought the volume in the washes and the number of washes might need to be increased. However, it had also been noted while processing several donors that even one wash after Treatment Media caused some disc material to be washed off during decanting, resulting in a less cohesive final product. The addition of extra washes and higher wash volumes could decrease the cohesiveness even further. Therefore, it was proposed to wait until after the washes to add the disc suspension to the treated and washed bone grindings. The second main reason for implementing this change was to decrease the processing time and additional training needed for Production personnel. By waiting to add the disc suspension until after the washes, the bone grindings can be decanted after each wash instead of centrifuged since the bone grindings do not retain as much media as the combined bone grindings and disc. By avoiding multiple centrifugation steps, Production personnel decants from the bone grindings for each wash step, which are familiar processing steps already performed. The time saved in the process by making this change could be as much as 10 minutes per wash, totaling 50 minutes saved during processing.

The one concern with this process change is that the disc material will no longer be washed in the Treatment Media containing antibiotics and antimycotic. However, the discs do go through an IPA decontamination shortly before homogenizing and placing in a jar for cooling. Therefore, there is minimal manipulation between decontamination and combining the discs with bone grindings, thereby negating the need for the Treatment Media soak.

The Process 2 alterations were first performed using donor W437523000564 to test the feasibility of performing the updated process (Table 20). In this process, the discs were homogenized in Plasma-Lyte with 10% DMSO. The XXXF bone grindings were washed three times in 10% DMSO in Plasma-Lyte after the Treatment Media shake. The washes were performed with a 1:2 ratio of bone grinding mass to 10% DMSO in Plasma-Lyte mass. After this, the disc suspension was added to the bone grindings, followed by the addition of 10% DMSO in Plasma-Lyte, which was added at a 1:1 ratio of the combined mass of disc suspension and bone grindings to the mass of 10% DMSO in Plasma-Lyte. All this material was shaken for 10 minutes at 150 RPM before being centrifuged at 1000×g for 10 minutes. This centrifugation step was switched back to 5 minutes for the Mock runs and final cleanroom process Verification runs (the centrifugation during Development runs was given extra time as a failsafe to ensure the success of centrifugation, even though it had already been performed many times at 5 minutes; the switch back to 5 minutes for future runs was made to save time while processing). The final product was then decanted, removed from the centrifuge tubes, mixed with the high torque stirrer, and packaged.

TABLE 20
Final Development Run (Process 2) Testing

| Study # | Donor ID | Process Changes | Testing Performed | Experimental Outcomes |
|---|---|---|---|---|
| 49 | W437523000564 | 1. Process 2 Feasibility | A. Post-thaw | Confirmed cohesive post-thaw |
| | | | cohesiveness | product with new process |
| 50 | W437523000578 | 1. Disc suspension | A. Primary digest | Confirmed presence of viable |
| | | added to bone | viability | cells; conditionally confirmed non- |
| | | grindings after 5 | B. Outgrowth | immunogenicity with the need to |
| | | post-treatment | C. MLR | re-test; confirmed presence of |
| | | washes | D. Growth factors | target growth factors; external |
| | | 2. DMSO added only in | E. B/F testing | testing lab confirmed passing B/F |
| | | final two washes | | growth testing |
| 51 | W437523000592 | 1. Disc suspension | A. Primary digest | Confirmed presence of viable |
| | | added to bone | viability | cells; conditionally confirmed non- |
| | | grindings after 5 | B. Outgrowth | immunogenicity with the need to |
| | | post-treatment | C. MLR | re-test; confirmed presence of |
| | | washes | D. Growth factors | target growth factors; external |
| | | 2. DMSO added only in | E. B/F teshing | testing lab confirmed passing B/F |
| | | final two washes | | growth testing |
| 52 | W437523000595 | 1. Disc suspension | A. Primary digest | Confirmed presence of viable |
| | | added to bone | viability | cells; conditionally confirmed non- |
| | | grindings after 5 | B. Outgrowth | immunogenicity with the need to |
| | | post-treatment | C. MLR | re-test; confirmed presence of |
| | | washes | D. Growth factors | target growth factors; external |
| | | 2. DMSO added only in | E. B/F testing | testing lab confirmed passing B/F |
| | | final two washes | | growth testing |

Three additional donors (W437523000578, W437523000592 and W437523000595) were processed with the Process 2 alterations, but with some changes from the feasibility test. These changes included a better way to calculate DMSO added to the discs when homogenizing (explained below in section IV.C.15.e), 5 total washes of the bone grindings post-Treatment Media shake (with shake times of 1, 1, 1, 20, and 1 minutes respectively), and 10% DMSO with Plasma-Lyte added only in the final two of the five washes, while just Plasma-Lyte was added in the first three. Similar to what was stated with the process alterations for the Process 1 Development runs, the longer wash in the middle (20 minutes) exists to allow more time for the antibiotics and antimycotic to elute from the product, so that samples are able to pass B/F testing and not cause false negatives during sterility testing.

For Process 2, the discs need to be homogenized to create a suspension with 10% DMSO. This is because the final wash needs to be in liquid at 10% DMSO, which was shown in processing to create conditions to give the highest viability for cells post-thaw. If the disc suspension without DMSO was added to the bone grindings, along with 10% DMSO in Plasma-Lyte, it would dilute the 10% DMSO in Plasma-Lyte solution, causing a decrease in the DMSO concentration. By having the media added at 10% DMSO along with the disc suspension at 10% DMSO with the bone grindings, the DMSO concentration remains at 10%. The best way to get the disc suspension to 10% DMSO, without simply adding a calculated amount of 100% DMSO straight into the discs in the blender, is to first add Plasma-Lyte to the discs to get the mass up to half the mass needed, and then add 20% DMSO in Plasma-Lyte for the remaining half of the total mass needed. The procedure was built this way so that the Plasma-Lyte and 20% DMSO could be prepared during media preparation. Below is an example to explain how this would look in the process.

### Equation:

(a) [Original mass of debrided discs]*2.5=[Total mass of disc and Plasma-Lyte]
(b) [Total weight of disc and Plasma-Lyte]−[Original mass of debrided discs+residual Plasma-Lyte mass post-decontamination]=[Total mass of Plasma-Lyte to add]
(c) [Original mass of debrided discs]*2.5=[Total mass of 10% DMSO in Plasma-Lyte to add]

### Example

(a) There were 50 g of disc debrided from a donor.
(b) [50 g of original debrided disc]*2.5=[125 g of disc and Plasma-Lyte in final suspension]
(c) After decontamination, the discs retained some Plasma-Lyte, and the mass of disc and residual Plasma-Lyte was 85 g total.
(d) [125 g of disc and Plasma-Lyte in final suspension]−[85 g original mass of debrided discs+residual Plasma-Lyte mass post-decontamination]=[40 g Total mass of Plasma-Lyte to add]
(e) 40 g of Plasma-Lyte would be added to the discs and residual Plasma-Lyte
(f) [50 g of original debrided disc]*2.5=[125 g Total mass of 10% DMSO in Plasma-Lyte to add]
(g) 125 g of 10% DMSO in Plasma-Lyte would be added to the 125 g of combined disc and Plasma-Lyte, for a total mass of 250 g of disc suspension.

One final change was made to Process 2 after the final Development Runs. During Mock and Verification runs conducted by Production personnel, Plasma-Lyte without DMSO was used for all the posttreatment media washes. 10% DMSO was only used for the final media added to the combined disc suspension (also with 10% DMSO) and bone grindings. This was done to minimize the amount of time the final product sits in DMSO prior to cryopreservation, to maximize cell viability.

### Example 19: Cryoprotective Agents

Existing graft products are cryopreserved using a 10% DMSO cryoprotective solution. Unless otherwise noted, the compositions described herein utilized 10% DMSO for continuity. However, an experiment was performed to test alternative CPA solutions for embodiments of the compositions described herein. The alternative CPAs included 10% propylene glycol (PG) as well as 10% DMSO or 10% PG solutions containing ice recrystallization inhibitors (IRI) (provided by PanTHERA CryoSolutions). IRIs inhibit the growth of ice crystals, which can improve the stability of cryopreserved cells.

Two (2) independent donors (W437523000495, W437523000500) were utilized for this experiment in which tissue was processed (Table 21). When ready for Treatment Medium, the tissue was divided into 6 groups each containing 25 g of tissue. A different cryosolution was added to each group at a ratio of 1.5 mL/g. The 6 groups were treated with their respective cryosolution for 60 minutes on a shaker (150 RPM) at ambient temperature. After treatment, the groups were centrifuged for 5 minutes at 1000×g and the cryosolutions were decanted. Each group of tissue was manually mixed prior to aliquoting into 5 mL tubes and frozen in a −86° C. freezer. After 4 to 6 days in the freezer, a sample from each group was thawed for primary digest to enumerate (via flow cytometry) the viable cells enzymatically digested from the 5 g/group tissue sample.

TABLE 21
CPA with IRA Testing

| Study # | Donor ID | Tested Variables | Experimental Groups | Experimental Outcomes |
|---|---|---|---|---|
| 38 | W437523000495 | 1. DMSO vs. | A. 10% DMSO with high IRI | 10% DMSO groups showed higher |
| | | PG | B. 10% DMSO with low IRI | viability post-thaw than all PG |
| | | 2. With and | C. 10% PG with high IRI | groups. 10% DMSO groups with |
| | | without IRI | D. 10% PG with low IRI | IRI showed higher viability than |
| | | | E. 10% DMSO | 10% DMSO alone group at time |
| | | | F. 10% PG | zero, but were comparable at 1- |
| | | | | month post-thaw |
| 39 | W437523000500 | 1. DMSO vs | A. 10% DMSO with high IRI | 10% DMSO groups showed higher |
| | | PG | B. 10% DMSO with low IRI | viability post-thaw than all PG |
| | | 2. With and | C. 10% PG with high IRI | groups. 10% DMSO groups with |
| | | without | D. 10% PG with low IRI | IRI showed higher viability than |
| | | IRI | E. 10% DMSO | 10% DMSO alone group at time |
| | | | F. 10% PG | zero, but were comparable at 1- |
| | | | | month post-thaw |

The 6 different cryosolutions were: 10% DMSO (Control 1), 10% PG (Control 2), and PanTHERA CPA/IRI solutions A, B, C, and D. Two (2) of the CPA/IRI solutions contained 10% DMSO with the 2 solutions containing different concentrations of IRIs, while the other 2 solutions contained 10% PG, similarly with 2 different concentrations of IRIs. The identities of CPA/IRI solutions A, B, C, and D were blinded. Additionally, a seventh group of tissue, which was treated with only Plasma-Lyte A (no CPA), was processed alongside the 6 cryosolution groups to serve as a negative control.

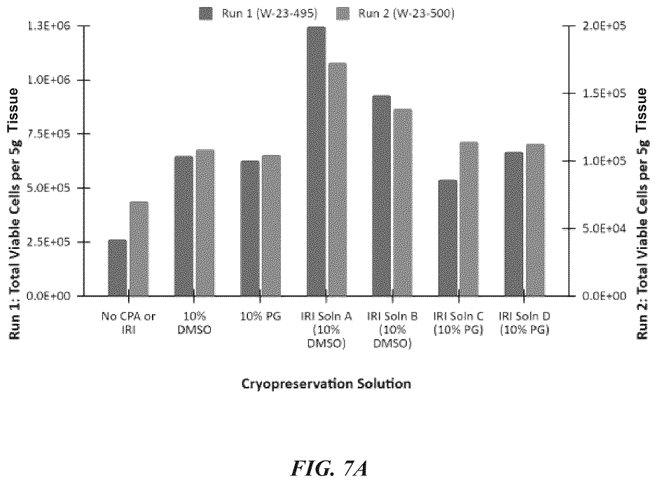

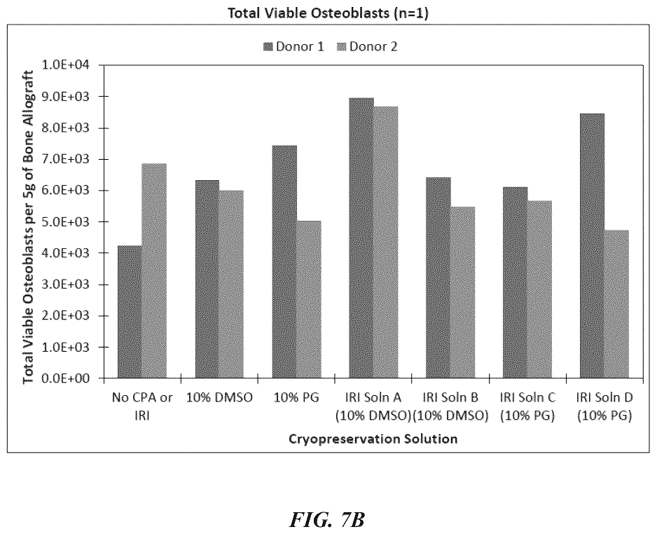

Time Zero: The post-thaw samples underwent primary digest (5 g/group) to enzymatically digest the cells off the bone and tissue. The resulting cells were assessed via flow cytometry to enumerate total viable cells and osteoblasts (Table 22 and FIGS. 7A-7B). The trends across cryosolution groups were consistent between both donors tested. 10% PG was comparable to 10% DMSO. IRI solutions A and B, which were later revealed to contain 10% DMSO, showed marked improvement compared to 10% DMSO alone. IRI solutions C and D, which were later revealed to contain 10% PG, showed little improvement compared to 10% PG alone.

TABLE 22
Time Zero, Post-Thaw Total Viable Cells and Osteoblasts (per 5 g)
Following Treatment with Different Cryopreservation Solutions.

| | Run 1 (W-23-495) | | | | Run 2 (W-23-500) | | | |
|---|---|---|---|---|---|---|---|---|
| | | | | Total | | | | Total |
| Cryo- | | | | Viable | | | | Viable |
| preservation | Total Viable Cells per 5 g | | | Osteoblasts | Total Viable Cells per 5 g | | | Osteoblasts |
| Solution | Aliquot 1 | Aliquot 2 | Average | per 5 g | Aliquot 1 | Aliquot 2 | Average | per 5 g |
| No CPA or | 3.0E+5 | 2.2E+5 | 2.6E+5 | 4.2E+3 | 7.7E+4 | 6.4E+4 | 7.0E+4 | 6.9E+3 |
| IRI | | | | | | | | |
| 10% DMSO | 6.3E+5 | 6.7E+5 | 6.5E+5 | 6.3E+3 | 1.3E+5 | 8.8E+4 | 1.1E+5 | 6.0E+3 |
| 10% PG | 6.2E+5 | 6.3E+5 | 6.3E+5 | 7.4E+3 | 1.2E+5 | 9.2E+4 | 1.0E+5 | 5.0E+3 |
| IRI Solution | 1.3E+6 | 1.2E+6 | 1.2E+6 | 9.0E+3 | 1.8E+5 | 1.7E+5 | 1.7E+5 | 8.7E+3 |
| A | | | | | | | | |
| (10% DMSO) | | | | | | | | |
| IRI Solution B | 9.5E+5 | 9.1E+5 | 9.3E+5 | 6.4E+3 | 1.4E+5 | 1.4E+5 | 1.4E+5 | 5.5E+3 |
| (10% DMSO) | | | | | | | | |
| IRI Solution | 5.2E+5 | 5.6E+5 | 5.4E+5 | 6.1E+3 | 1.3E+5 | 1.0E+5 | 1.1E+5 | 5.7E+3 |
| C | | | | | | | | |
| (10% PG) | | | | | | | | |
| IRI Solution | 6.9E+5 | 6.5E+5 | 6.7E+5 | 8.5E+3 | 1.4E+5 | 8.7E+4 | 1.1E+5 | 4.7E+3 |
| D | | | | | | | | |
| (10% PG) | | | | | | | | |

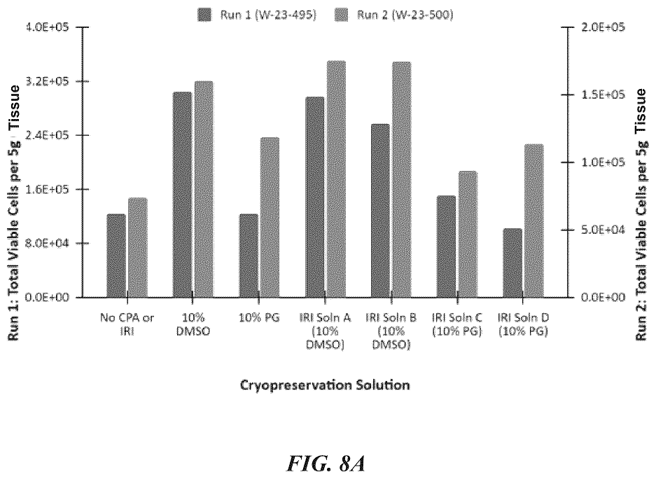

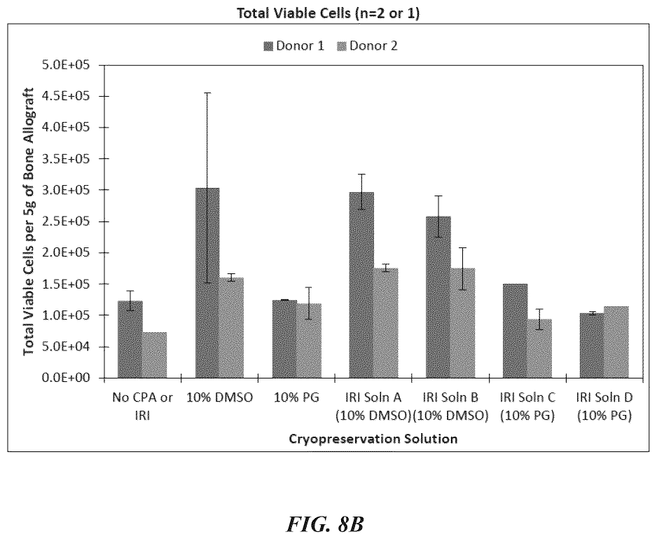

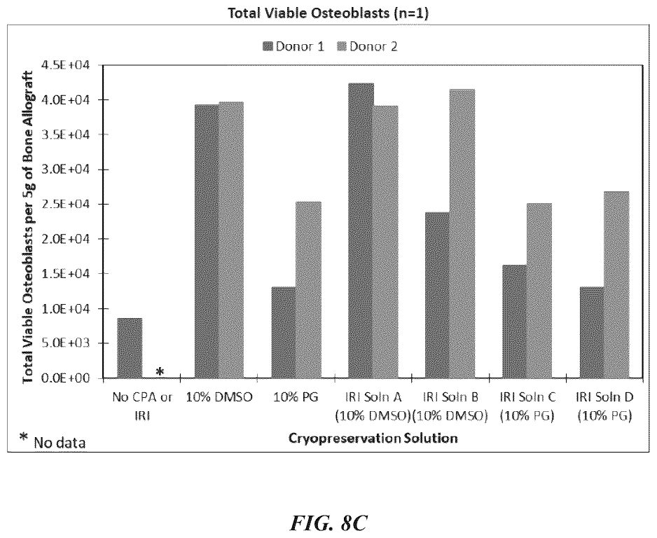

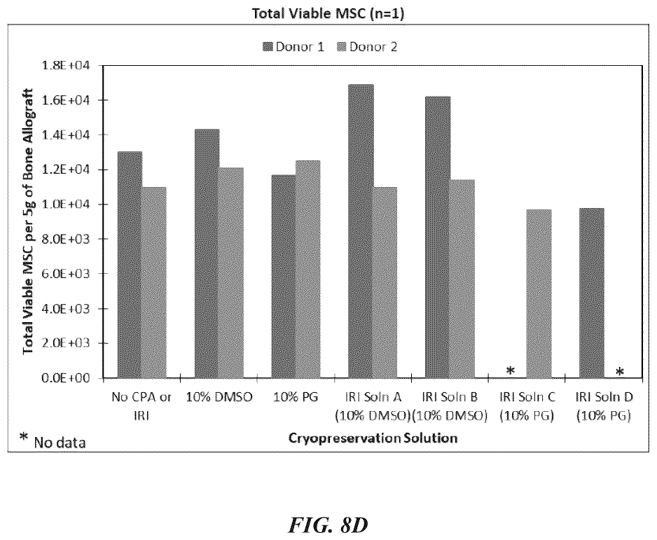

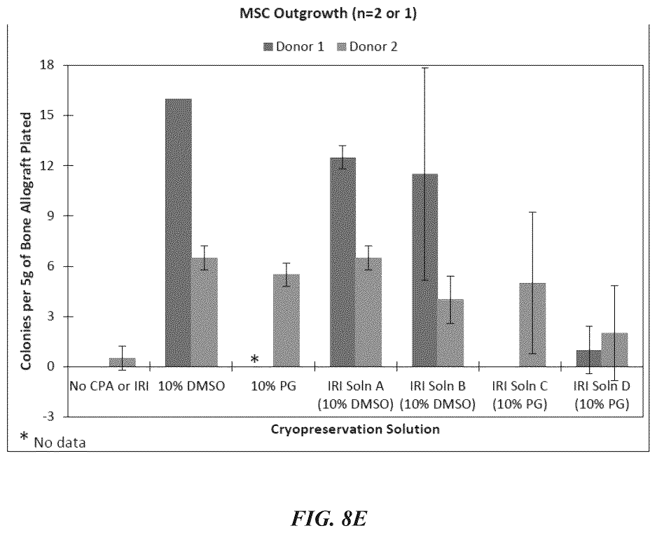

1-Month: Duplicate samples from the same donors and experimental groups were thawed and tested after 1 month of −86° C. storage to reassess cryosolution impact after a longer storage duration. The 1-month samples were processed the same as the Time Zero samples, but for each set of duplicate samples, one sample was stained for osteoblasts while the other sample was stained for MSC (Table 23 and FIGS. 8A-8E). After 1 month of storage, the 10% DMSO groups (with and without IRI) demonstrated higher post-thaw cell viability compared to the 10% PG groups (with and without IRI). Interestingly, IRI solutions A and B, which contained 10% DMSO, did not have the same marked improvement over 10% DMSO alone that was seen at Time Zero. Total viable cells and total viable osteoblasts were most impacted by the different cryosolutions; total viable MSC showed less change across groups.

TABLE 23
One Month, Post-Thaw Total Viable Cells and Osteoblasts (per 5 g)
Following Treatment with Different Cryopreservation Solutions.

| | Run 1 (W-23-495) | | | | | Run 2 (W-23-500) | | | | |
|---|---|---|---|---|---|---|---|---|---|---|
| | | | | Total | | | | | Total | |
| | | | | Viable | Total | | | | Viable | Total |
| Cryo- | Total Viable Cells per 5 g | | | Osteo- | Viable | Total Viable Cells per 5 g | | | Osteo- | Viable |
| preservation | Sample | Sample | | blasts | MSC | Sample | Sample | | blasts | MSC |
| Solution | 1 | 2 | Average | per 5 g | per 5 g | 1 | 3 | Average | per 5 g | per 5 g |
| No CPA or | 1.4E+5 | 1.1E+5 | 1.2E+5 | 8.6E+3 | 1.3E+4 | NA | 7.4E+4 | 7.4E+4 | NA | 1.1E+4 |
| IRI | | | | | | | | | | |
| 10% DMSO | 2.0E+5 | 4.1E+5 | 3.0E+5 | 3.9E+4 | 1.4E+4 | 1.7E+5 | 1.6E+5 | 1.6E+5 | 4.0E+4 | 1.2E+4 |
| 10% PG | 1.2E+5 | 1.3E+5 | 1.2E+5 | 1.3E+4 | 1.2E+4 | 1.0E+5 | 1.4E+5 | 1.2E+5 | 2.5E+4 | 1.3E+4 |
| IRI Solution | 2.8E+5 | 3.2E+5 | 3.0E+5 | 4.2E+4 | 1.7E+4 | 1.8E+5 | 1.7E+5 | 1.8E+5 | 3.9E+4 | 1.1E+4 |
| A (10% | | | | | | | | | | |
| DMSO) | | | | | | | | | | |
| IRI Solution | 2.4E+5 | 2.8E+5 | 2.6E+5 | 2.4E+4 | 1.6E+4 | 2.0E+5 | 1.5E+5 | 1.8E+5 | 4.2E+4 | 1.1E+4 |
| B (100% | | | | | | | | | | |
| DMSO) | | | | | | | | | | |
| IRI Solution | NA | 1.5E+5 | 1.5E+5 | 1.6E+4 | NA | 1.1E+5 | 8.2E+4 | 9.4E+4 | 2.5E+4 | 9.7E+3 |
| C (10% PG) | | | | | | | | | | |
| IRI Solution | 1.3E+5 | 1.3E+5 | 1.3E+5 | 1.3E+4 | 9.8E+3 | 1.0E+5 | NA | 1.0E+5 | 2.7E+4 | NA |
| D (10% PG) | | | | | | | | | | |

With the mixed results from testing only two donors for a limited amount of time, additional testing has been planned to verify the ability of IRIs to increase post-thaw cell viability. Since the PG groups did not demonstrate improved post-thaw viability compared to DMSO groups, additional testing on more donors will focus on 10% DMSO with varying concentrations of IRIs.

Example 20: Aliquoting into Pouches

To reduce variability and ensure sufficient product is packaged into each pouch, VBX will be aliquoted by mass. Referencing a product density maximum of 1.3 g per cc, each pouch will be weighed as it is being filled to achieve the target fill volume (e.g., ≥6.5 g for 5 cc). Additionally, setting an acceptable upper limit (e.g., ≤7.0 g for 5 cc) will minimize product waste while maintaining a narrow range of fill volumes.

### Example 21: Packaging Hold Time Stability

VBX packaging hold time stability was previously evaluated. Compositions subjected to post-decanting hold times of up to 3 hours still met all established acceptance criteria for quality, safety, and potency (OHC-VALS-QC-0006 Cellular Bone Matrix Hold Time Post-Decanting Stability Study Report). Some differentiating features between current graft compositions (100% VB) and compositions described herein includes the addition of IVD (85% VB, 15% IVD). The addition of the non-viable IVD component does not impact the existing viable VB component. Therefore, the presence of IVD would have negligible impact on post-decanting packaging hold time stability. As a result, the acceptable time between decanting the final wash and transferring packaged compositions into the freezer will be kept the same, thus minimizing processing differences between the two VBX products.

### Example 22: Methods of Use

MSC outgrowth was tested using the same procedure used for VBX compositions: 0.3 g/well of VBX in triplicate wells of a 6-well plate with 2 mL/well MSC Culture Medium. However, the compositions described herein increased cohesiveness made it difficult to disperse the VBX within a well. This may have caused the lack of outgrowth observed, even when up to 1.2 g/well of VBX were plated. To increase cell access to the plate, the cells within VBX were enzymatically digested off the tissue and the resulting primary digest was plated.

After transferring to plating digest, there were still issues with MSC colony formation. The Development team believed these continued issues to be caused by digest debris now covering the surface of the plate making it difficult for cells to adhere. To ensure this was digest debris, and not cell functionality issues, primary digest was plated at varying densities on 145 cm2 petri dishes to have approximately fifteen times the surface area for cells to adhere even in the presence of debris. The media volume used was adjusted proportionally to 30 mL to ensure cells were adequately covered. To each petri dish, 3 g, 6 g, and 9 g samples were plated alongside whole, undigested VBX samples of equal mass. All samples were from donor W437523000482 to avoid donor variability. Results are visible in Table 24 below, with digested samples averaging 3.44 with a standard deviation of 1.02 colonies per gram of sample. Undigested samples displayed no growth at every plating density.

TABLE 24
Whole vs Digested VBX Outgrowth Results
(Total Enumerated Colonies)

| | |
|---|---|
| ID: W437523000482 | Plating Density |

| Sample Type | 3 g/plate | 6 g/plate | 9 g/plate |
|---|---|---|---|
| Whole VBX | 0 | 0 | 0 |
| Digested VBX | 11 | 14 | 39 |

After growth was demonstrated utilizing a larger surface area, outgrowth of the primary digest was performed in a 6-well plate with donors: W437523000396 W437523000415, W437523000578, W437523000592, W437523000595, W437524000632, W437524000645. Results for these donors are in Table 25 below. The no growth result for donor W437523000592 is likely due to outgrowth assay limitations since this donor did not have reduced viable cell counts when tested via flow cytometry.

TABLE 25
Digested Outgrowth Results

| | Total | Average | |
|---|---|---|---|
| | Colonies | Colony | Standard |
| Donor ID | Counted | per Well | Deviation |
| W437523000396 | 11 | 3.67 | 1.15 |
| W437523000415 | 2 | 0.67 | 1.15 |
| W437523000578 | 9 | 3 | 1 |
| W437523000592 | 0 | 0 | 0 |
| W437523000595 | 7 | 2.33 | 1.15 |
| W437524000632 | 24 | 8 | 5.29 |
| W437524000645 | 6 | 2 | 1.73 |

### Example 23: Density (Mass to Volume Ratio)

Density testing was conducted to allow for additional verification methods to packaging volume by knowing equivalent mass of the product. Due to the capability of the compositions to retain water, both dry and wet density was measured. Wet density was measured to have an average of 1.207, and a standard deviation of 0.018 across seven samples (Table 26). Dry density was measured to have an average of 1.303, and a standard deviation of 0.049 across five samples (Table 27).

TABLE 26
Wet Density Measurements

| | Sample | Sample | |
|---|---|---|---|
| | Mass | Volume | Density |
| Donor ID | (g) | (mL) | (g/mL) |
| W437523000322 | 6.920 | 5.70 | 1.214 |
| W437523000315 | 4.917 | 4.00 | 1.229 |
| W437523000326 | 7.494 | 6.15 | 1.219 |
| W437523000354 | 7.485 | 6.15 | 1.217 |
| W437523000369 | 7.609 | 6.45 | 1.180 |
| W437523000396 | 7.694 | 6.40 | 1.202 |
| W437523000415 | 6.586 | 5.55 | 1.187 |

| | |
|---|---|
| Average | 1.207 ± 0.018 |

TABLE 27
Dry Density Measurements

| | Sample | Sample | |
|---|---|---|---|
| | Mass | Volume | Density |
| Donor ID | (g) | (mL) | (g/mL) |
| W437523000315 | 2.951 | 2.30 | 1.283 |
| W437523000354 | 5.120\| | 3.85 | 1.330 |
| W437523000369 | 3.691 | 3.00 | 1.230 |
| W437523000396 | 3.813 | 2.90 | 1.315 |
| W437523000415 | 4.009 | 2.95 | 1.359 |

| | |
|---|---|
| Average | 1.303 ± 0.049 |

For wet density testing, samples were thawed, then weighed immediately with no additional liquid removal. After this, samples were then weighed on an analytical balance, and placed into a 25 mL graduated cylinder in 10 mL of water to measure volume displacement. For dry density testing, thawed samples were flattened and pressed between dry wipes until no more liquid could be pressed out. After this, the same mass measurements on an analytical balance, and volume measurement was done by volume displacement in a 25 mL graduated cylinder with 10 mL of water.

### Example 24: Immunogenicity

To test whether the product is immunogenic or not (does or does not stimulate an immune response), mixed lymphocyte reaction (MLR) testing was conducted. The assay, originally structured by Xeno Diagnostics and further optimized in-house, consists of first isolating peripheral blood mobilized cells (PBMC) from donor whole blood, provided by Versiti Blood Center. These cells are stained with CFSE and frozen. When a sample of VBX is ready, the PBMC are thawed and plated in 24-well plates at 1.2×10⁶ cells/mL in RPMI-FBS medium. The test wells and controls are done in triplicate wells. There are three control groups: a positive control that has 150 ng/mL CD3 antibody, a negative control with 200 nM Dexamethasone (lymphocyte inhibitor), and a control with just cells in RPMI-FBS medium. The test wells have 0.25 g/well of sample that is pressed into the bottom of the well to cover the entire surface area of the bottom. The 1 mL of PBMCs with 1.2×10⁶ cells/mL are pipetted on top of the compositions in each well. The plates containing the controls and test groups were incubated (37° C., 5% CO₂) for 7 days to allow for the potential for T-cells to mount a response and proliferate.

The results are based on the percentage of proliferating and nonproliferating cells divided by the percentage of non-proliferating cells, for each control and test group. The final ratio is the result from each group compared to the result from the negative control. The results for the MLR test can be seen in Table 28.

TABLE 28
MLR Immunogenic Testing

| | | Ratio Compared to |
|---|---|---|
| Date Tested | Group Tested | Negative Control |
| 8 Nov. 2023 | No Stimulation Control | 1.0021 |
| (PBMC Donor #13) | Dexamethasone Negative Control | 1.0000 |
| | CD3 Positive Control | 9.9522 |
| | W-23-354 | NA* |
| | W-23-369 | 1.0011 |
| | W-23-396 | 1.0008 |
| | W-23-415 | 1.0003 |
| 8 Nov. 2023 | No Stimulation Control | 1.0023 |
| (PBMC Donor #3) | Dexamethasone Negative Control | 1.0000 |
| | CD3 Positive Control | 8.3800 |
| | W-23-354 (Repeat test) | 1.0006 |
| | W-23-396 (Verifying new batch of PBMC) | 1.0009 |
| 17 Jan. 2024 | No Stimulation Control | 1.0003 |
| | Dexamethasone Negative Control | 1.0000 |
| | CD3 Positive Control | 1.0024** |
| | W-23-483 | 1.0034 |
| | W-23-484 | 1.0020 |
| | W-23-544 | 1.0089 |
| | W-23-546 | 1.0034 |
| | W-23-558 | 1.0066 |
| | W-23-578 | 1.0057 |
| | W-23-592 | 1.0065 |
| | W-23-595 | 1.0047 |
| 23 Feb. 2024 | No Stimulation Control | 1.0005 |
| | Dexamethasone Negative Control | 1.0000 |
| | CD3 Positive Control | 1.0004** |
| | W-23-546 | 0.9995 |
| | W-23-578 | 1.0074 |
| | W-23-592 | 1.0013 |
| | W-23-595 | 1.0013 |
| | W-24-632 | 0.9999 |
| | W-24-645 | 1.0015 |

Note:
*indicates flow cytometer failure for multiple samples of this donor.
**indicates CD3 positive control failed to have increased replication. PBMC donor was shown to grow in CD3 positive control for test on 8 Nov. 2023. All donors tested with this PBMC donor with failed CD3 positive control will be re-tested.

While none of the samples showed a significant increase in proliferation, two of the tests had positive controls that did not grow, even when stimulated with CD3 antibody. This makes the data from all the samples tested from those two runs questionable because if the T-cells in the PBMC could not grow even when stimulated, then it would be difficult to say that they could have grown if one of the samples was immunogenic. Therefore, although it is highly unlikely since MLR test results were clear, there is the potential for false negatives in this data set. As such, the donor samples tested during the two tests with no significant CD3 Positive Control growth will be re-tested in future Development work.

### Example 25: Primary Digest and Flow Cell Viability

Primary digest and flow cytometry were performed on multiple post-thaw samples to determine cell viability and get specific viable cell counts of osteoblasts and MSC. For all the samples tested via primary digest and flow cytometry, samples were removed from cryopreservation, thawed in a 37° C. water bath, and 5 g samples were weighed out for each experimental group. All these samples were then digested in Whirl-Pak bags or 50 mL conical tubes, using collagenase and protease, while heated in a water bath for 40 minutes at 37° C. and subsequently shaken and heated in a shaking incubator for 80 minutes at 40° C. Stemulate hPL was used to neutralize the collagenase and protease, and then grindings were filtered and washed multiple times. The remaining cells were stained and tested for flow cytometry, using markers to test for live cells (Calcein+DAPI−), live osteoblast cells (Calcein+DAPI−CD45−CD90+TNAP+), and live MSC (Calcein+DAPI−CD45−CD90+CD166). Calcein staining occurred in a 37° C. incubator for 30 minutes, while all other staining occurred for 15 minutes at ambient temperature. There were multiple washes between stains and the final samples were resuspended with DAPI stain.

The data from these tests to confirm the presence of viable cells is shown below in Table 29.

TABLE 29
Primary Digest and Flow Viability Testing

| Process | | | Live Cell | Live | Live |
|---|---|---|---|---|---|
| Iteration | Disc Decon. | Donor | Count/g | Osteoblasts/g | MSC/g |
| Process 1 | No IPA Disc Decon. | W-23-330 | 3.70E+05 | 3.00E+04 | 6.50E+03 |
| | | W-23-333 | 1.40E+05 | 4.90E+04 | 4.50E+04 |
| | | W-23-354 | 4.10E+05 | 3.00E+04 | 7.80E+03 |
| | IPA Disc Decon. | W-23-482 | 1.51E+05 | 9.18E+03 | 6.63E+03 |
| | | W-23-483 | 2.66E+05 | 1.17E+04 | 7.80E+03 |
| | | W-23-484 | 7.29E+05 | 1.81E+04 | 8.45E+03 |
| | | W-23-544 | 1.43E+05 | 8.09E+03 | 9.32E+03 |
| | | W-23-546 | 2.69E+05 | 5.68E+03 | 3.79E+03 |
| | | W-23-558 | 1.79E+05 | 6.41E+03 | 1.60E+03 |
| Process 2 | | W-23-578 | 2.49E+05 | 1.57E+04 | 8.74E+03 |
| | | W-23-592 | 3.97E+05 | 1.80E+04 | 1.07E+04 |
| | | W-23-595 | 2.51E+05 | 7.21E+03 | 4.08E+03 |
| | | W-24-632 | 1.28E+05 | 8.52E+03 | 5.83E+03 |
| | | W-24-645 | 2.37E+05 | 2.16E+04 | 8.82E+03 |

| | | | |
|---|---|---|---|
| Average | 2.80E+05 ± | 1.71E+04 ± | 9.65E+03 ± |
| | 1.60E+05 | 1.23E+04 | 1.05E+04 |

### Example 26: Growth Factors

The compositions were evaluated for the presence of key growth factors that aid in the recruitment of resident cells for bone reformation and healing. Growth factors tested for included: BMP-2, BMP-4, BMP-7, BMP-9, FGF-1, FGF-2, osteoactivin, osteopontin, PDGF-BB, TNFa, and VEGF. For growth factor extraction, 330 mg of VBX were decalcified for 24 hours in extraction medium, centrifuged to remove large tissue, then the supernatant was filtered to remove fine tissue particulate. The supernatant was then washed multiple times through a 3 kDa filter to replace all extraction medium with Phosphate Buffered Saline (PBS) while the filter retained the target growth factor protein. Triplicate extractions were performed for each of 10 donors to produce 29 samples (one sample was lost in processing).

All extracted growth factors were tested using Luminex FLEXMAP 3D discovery assays. The Luminex reports results in picogram per milliliter (pg/mL) of sample tested; however, all data was converted to picogram of protein per gram of VBX used for the extraction. The averages and standard deviations for growth factors quantified are presented in Table 30, which notes sample accuracy for select growth factors.

TABLE 30
Growth Factors Averages and Standard Deviations

| | | Standard |
|---|---|---|
| | Average | Deviation |
| | (pg/g of | (pg/g of |
| Growth Factor | allograft) | allograft) |
| BMP-2 | 74.51 | 38.30 |
| BMP-7 | 2330.27 | 728.88 |
| BMP-9* | 1.45* | 0.67* |
| FGF-1 | 386.53 | 105.60 |
| FGF-2 | 6.58 | 2.77 |
| Osteoactivin | 3518.36 | 788.16 |
| Osteopontin | 4115.28 | 3297.36 |
| PDGF-BB | 147.25 | 49.46 |
| TNFα* | 4.71* | 1.72* |
| VEGF* | 13005.97* | 7297.47* |

*TNFα had 29 samples under the lowest standard, BMP-9 had 28 samples under the lowest standard, and VEGF had 25 samples that exceeded the highest standard

### Example 27: Thaw Time

Thaw Method for End Use: In alignment with similar existing commercial products, and for uniformity across the VBX product line, the end user thaw method is to preheat 2 L of sterile water or saline to 35 to 39° C. The preheated water/saline is then transferred into a sterile basin. The ported pouch containing the compositions is submerged in the water basin within 5 minutes from being removed from frozen storage (≤60° C.; e.g., dry ice, ultra-cold freezer.) If the end user needs to thaw more than one pouch, it is recommended to prepare a sterile basin for each pouch, and additional basins would need to be prepared.

Time Needed to Thaw: Time required to thaw the compositions in accordance with the thaw method for end user can vary. During development testing, 3 cc aliquots showed a range of 2 to 5 minutes, and 5 cc aliquots ranged from 3 to 5 minutes. For both aliquot sizes, times were extended by two key factors: excess air within packaging pouch, and how well distributed the product was across the package. For both aliquot sizes tested, thaw time did not exceed 5 minutes. The end user must confirm the allograft is completely thawed prior to use as exact thaw times will vary.

For the existing VBX product, excess air is removed from the packaging, and the product is to be evenly distributed within packaging. This means no additional packaging process changes are required to try and minimize thaw times when packaging.

Thawing in Electric Water Bath: Compared to a basin containing pre-heated water, a 35 to 39° C. electric water bath was shown to speed up thaw times, if anything, for the existing VBX product (OHCDEV-R-0003). A slightly accelerated thaw time does not pose a risk to the VBX product. Therefore, use of a 35 to 39° C. electric water bath is also acceptable for the compositions described herein.

### Example 28: Post-Thaw Benchtop Stability

Benchtop Stability was determined by allowing thawed samples to rest on the benchtop for set durations prior to testing. Benchtop stability was assessed using three development runs packaged at 5 cc, and tested at timepoints of 0-, 1-, 2-, and 3-hours post-thaw. Samples were thawed in accordance with the thaw method for end user. Stability was measured by enumerating viable cells via flow cytometry after digesting the post-thaw samples with DE-100 Collagenase.

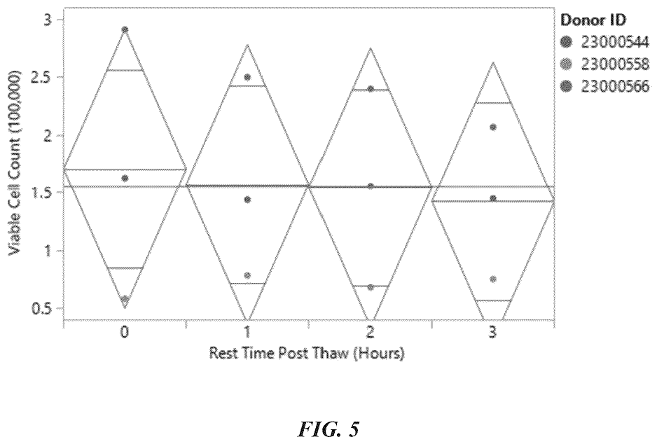

After data collection, cell viability counts at each time point were analyzed via a one-way ANOVA to check for statistical variance across the four different timepoints. A graph displaying the benchtop stability data is shown in FIG. 5. Across all time points no statistical difference was shown (p=0.9842) and individual sample viability is as shown in Table 31.

TABLE 31
Viable Cell Counts

| ID | Time 0 | Time 1 | Time 2 | Time 3 |
|---|---|---|---|---|
| W437523000558 | 5.88E+04 | 7.88E+04 | 6.84E+04 | 7.56E+04 |
| W437523000544 | 1.63E+05 | 1.44E+05 | 1.56E+05 | 1.45E+05 |
| W437523000546 | 2.91E+05 | 2.50E+05 | 2.40E+05 | 2.07E+05 |

### Example 29: Production Mock Runs

Three donor spines were processed (W437523000610, W437524000632, and W437524000645) (Table 32).

TABLE 32
Mock Runs Testing

| Study # | Donor ID | Process Changes | Testing Performed | Experimental Outcomes |
|---|---|---|---|---|
| 53 | W437523000610 | 1. Four post- | A. None (donor several | NA |
| | | treatment washes | days over CIT) | |
| | | of bone grindings | | |
| 54 | W437524000632 | 1. Four post- | A. Primary digest | Confirmed presence of viable |
| | | treatment washes | viability | cells; conditionally confirmed |
| | | of bone grindings | B. Outgrowth | non-ininnmogenicity with the |
| | | | C. MLR | need to re-test; confirmed |
| | | | D. Growth factors | presence of all necessary |
| | | | | growth factors |
| 55 | W437524000645 | 1. Four post- | A. Primary digest | Confirmed presence of viable |
| | | treatment washes | viability | cells; conditionally confirmed |
| | | of bone grindings | B. Outgrowth | non-immunogenicity with the |
| | | | C. MLR | need to re-test, confinned |
| | | | D. Growth factors | presence of all necessary |
| | | | | growth factors |

There were two differences between the process used for the Mock runs and the subsequent Verification runs and future Validation runs. The first difference is that only 4 washes were used post-treatment media soak. The fifth wash was removed for these three runs because a “fifth wash” was already happening when the disc suspension and bone grindings were combined with 10% DMSO in Plasma-Lyte. However, due to initial complications observed with B/F testing for the ongoing production, the additional wash was added back to the process for Verification runs and future Validation runs. The second difference was that each wash step included a shake on the shaker at 125 RPM, instead of 150 RPM. It was observed that with a 2:1 media mass to bone grindings mass ratio, with larger donors, that the bone grindings at the very bottom of the bowl did not move with minimal dead space in the container to allow for proper shaking. The speed was increased to 150 RPM for all the washes completed during Verification runs and future Validation runs.

### Example 30: Final Process

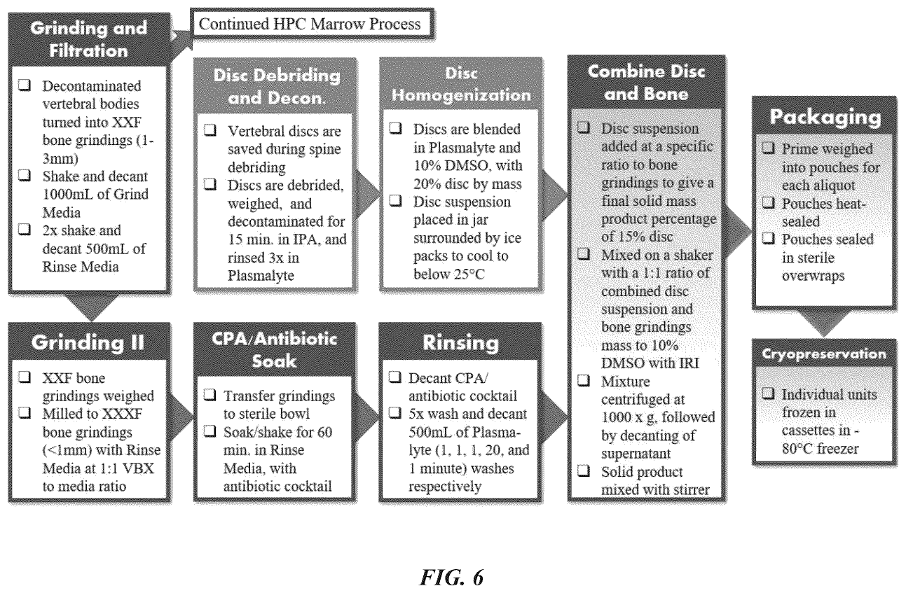

A non-limiting example final process is as outlined in FIG. 6. A summary of that process is as follows:

### Media Creation:

i. Prepare 500 mL of Treatment Medium, by adding 429 mL of Plasma-Lyte A to a bottle, along with 50 mL of 25% HSA. Continue by mixing 250 mg Vancomycin (¼ bottle of 1 g-5 mL), 50 mg Voriconazole (¼ bottle of 200 mg-5 mL), 50 mg Polymyxin B (1 full bottles of 500,000 units-10 mL), and 50 mg Gentamicin (1.25 mL from 40 mg/ml bottle). Run all media through a sterile media filter.
ii. Prepare 500 mL of Rinse Medium by combining 450 mL of Plasma-Lyte A with 50 mL of 25% HSA. Run all media through a sterile media filter.
iii. Prepare 6 L of Plasma-Lyte A by transferring each 1 L bag of Plasma-Lyte into a 1 L sterile bottle.
iv. Prepare 1 L of Plasma-Lyte A with 10% DMSO and 2.86 mg/mL IRI. First, prepare 90% DMSO in sterile water and then reconstitute 2.99 g of IRI in 115 mL of the 90% DMSO. Combine 110 mL of the IRI in 90% DMSO with 890 mL of Plasma-Lyte in a 1 L bottle. Sterile filter this solution via 0.2 um vacuum filtration.
v. Prepare 250 mL of 20% DMSO in Plasma-Lyte A, by pouring 200 mL of Plasma-Lyte A and 50 mL of DMSO into a 250 mL sterile bottle. Pour the bottle through a sterile media filter.

### Disc Homogenization:

vi. Production personnel slice whole intervertebral discs from between vertebral bodies during debridement.
vii. Place the discs in a rectangular stainless-steel pan. Assess discs for degeneration. Cut degenerated tissue, cartilaginous endplate, and other debris from full discs.
viii. Weigh out sliced and debrided discs. If there is less than 40 g of disc, do not proceed with processing. Do not process more than 100 g of disc.
ix. Place the discs in a sterile bowl and add an entire 300 mL of 70% isopropyl alcohol (IPA). Close the bowl and place it on the shaker for 15 minutes at 125 RPM at ambient temperature.
x. After the shake, decant the IPA over a sieve into a stainless-steel catch pan used as a waste container. Using forceps, remove the discs from the first sterile bowl and place them in a new sterile bowl.
xi. Add 300 mL of Plasma-Lyte A to the new bowl containing the discs. Place the lid on the bowl and place the bowl on the shaker for 1 minute at 125 RPM. Pour the Plasma-Lyte over the sieve into the circular steel pan. Repeat two more times, with an exception on the third shake: place the bowl on the shaker for 5 minutes at 125 RPM before decanting.
xii. Add discs to Eberbach stainless steel blending container (Heavy Duty Blender container) with Plasma-Lyte at a 1:1.5 disc to Plasma-Lyte ratio by mass. The total mass needed in the blending container can be found by multiplying the initial pre-rinse mass of discs by 2.5. After zeroing the scale with the blending container on it, and adding the discs and residual Plasma-Lyte to the blending container, pour additional Plasma-Lyte into the container until reaching the calculated total mass of disc and Plasma-Lyte. Then add 20% DMSO in Plasma-Lyte at a ratio of 1:2.5 disc to 20% DMSO in Plasma-Lyte ratio by mass. The total final mass added now should be 5 times the disc mass. Place the screw top lid on the container, remove from the BSC, and place the container on the Waring blender (Heavy Duty Blender).
xiii. Homogenize discs by running the Waring blender (Heavy Duty Blender) on “Low” for 30 seconds, “Medium” for 30 seconds, and then “High” for 90 seconds. Let sit for 30 to 60 seconds. Repeat the same blends of “Low” for 30 seconds, “Medium” for 30 seconds, and then “High” for 90 seconds. Repeat the same blend setting one more time. In the BSC, pour the disc suspension into a 1.25 L stainless steel sterile jar. Scrape out the blending container using a sterile stainless-steel spatula. Inspect the disc suspension as it is poured and scraped to ensure there are no large (≥0.5 cm diameter) pieces of disc. A point of reference is to ensure there are no disc pieces much larger than any of the bone grinding. If there are large pieces upon immediate inspection, remove large pieces and discard before proceeding. Place the lid on the 1.25 L jar. Remove the gel ice packs from the −20° C. freezer and place them around the jar. Place the jar into the back corner of the BSC.

### Further Bone Grinding: XXXF Bone Grindings

xiv. Production personnel take vertebral bodies and debride, grind (using XXF blade), rinse, and filter out bone marrow from them. The tissue team will do an additional decant and pipetting of any remaining liquid into a waste bowl. Place the decanted XXF bone grindings in a new sterile bowl and weigh the bone grindings.
xv. Mill bone grindings to XXXF size, while washing them through the bone mill into a sterile bowl, using 1:1 ratio of bone grindings mass in grams to Rinse Media (containing 2.5% HSA in Plasma-Lyte) mass in grams. Using a funnel, place ⅓ to ½ the grindings in the mill housing at a time. Pull material from the mill blade housing as it collects using a pair of forceps. Wash Rinse Media through the top of the mill housing only after all the material for each ⅓ or ½ has passed through the milling blade. Decant supernatant from XXXF bone grindings, pipette out any excess supernatant. Weigh out XXXF bone grindings into a new sterile bowl.
xvi. Add Treatment Media (Plasma-Lyte containing 500 ug/mL Vancomycin, 100 ug/mL Voriconazole, 100 ug/mL Gentamicin, 100 ug/mL Polymyxin B, and 2.5% HSA) at a 1:1 ratio of VBX mass in grams to Treatment Media mass to the bowl of bone grindings. Place the sterile bowl with combined media, disc, and bone grindings on the shaker at 125 RPM for 1 hour.
xvii. Decant and pipette out the supernatant. Place bone grindings in a new sterile bowl. Add Plasma-Lyte at a 1:2 ratio of VBX mass in grams to Plasma-Lyte mass in grams. Place on the shaker for 1 minute at 150 RPM.
xviii. Repeat Step 4 two more times.
xix. Decant and pipette out the supernatant. Add Plasma-Lyte at a 1:2 ratio of VBX mass in grams to Plasma-Lyte mass in grams. Place on the shaker for 20 minutes at 150 RPM.
xx. Decant and pipette out the supernatant. Place bone grindings in a new sterile bowl. Add Plasma-Lyte at a 1:2 ratio of VBX mass in grams Plasma-Lyte mass in grams. Place on the shaker for 1 minute at 150 RPM.
xxi. Decant and pipette out the supernatant. Weigh the bone grindings.

### Combining Disc and Bone Grindings

xxii. Remove the cooling packs from around the disc suspension jar. Using an infrared thermometer, check the temperature of the disc suspension prior to use to ensure the temperature is at or below 25° C. Pour the disc suspension into a sterile bowl. Scrape out any remaining disc suspension from the jar using a sterile spatula. Weigh out the disc suspension.
xxiii. Add enough suspended disc to the sterile bowl of bone grindings to create a final product with 15% disc and 85% bone grindings by mass.
   (a) The amount of disc needed equals [mass of bone grinding]*[0.15/0.85].
   (b) Pure disc accounts for 20% of disc suspension, so the final mass of suspension needed is 5× the amount of disc from Step b. iii.
   (c) Final equation comes out to [mass of disc suspension]=[mass of bone grinding]*[15/17].
   (d) Note: If this calculation comes out to be more than the disc suspension measured, then a calculation to determine how much bone grindings should be added to the whole disc suspension must be done. In this case, add the bone grindings to the bowl with the disc suspension, instead of the other way around.
   (e) This equation is [mass of disc suspension]*[17/15]−[mass of bone grinding].
iii. Add 10% DMSO in Plasma-Lyte at a 1:1 ratio of total combined disc suspension and bone grindings mass to 10% DMSO in Plasma-Lyte. Place the bowl on the shaker for 10 minutes at 150 RPM.
iv. Using a funnel, pour the combined disc suspension and bone grindings equally into two or four 500 mL conical tubes (four conical tubes will be used if total mass of disc, bone grindings, and solution are over 1000 g). Make sure the liquid and solid material is evenly distributed between tubes (do not put all the media in one tube and all the solid material in the other(s)). Weigh the tubes to ensure they have mass within 5 g of each other.
v. Centrifuge the conical tubes at 1000× g for 5 minutes. Decant the supernatant into a waste container.
vi. Pull final product from 500 ml conical tubes and place in a sterile plastic bowl.
vii. Using the stand mixer (High Torque Stirrer) with the appropriate mixing head, mix the bone grindings and disc in the bowl at 1000 RPM for at least 3 minutes, until homogenous. Using a stainless-steel spatula, scrape off the mixing head and the walls of the jar to retain as much product as possible. Analyze the final product for large clumps or inconsistent or unmixed tissue.
viii. Using a forceps, aliquot into pouches, confirm the weight achieves the desired volume utilizing a density of 1.3 g/cc, seal the inner pouch, place the inner pouch inside the outer pouch, seal the outer pouch, and place final product units in an ultra-low temperature (≤−70° C.) freezer.

Conclusions: Development testing generated the production process and verified process quality for the product. Key process steps and parameters were studied and identified. Viable cells were confirmed in the final product via outgrowth and flow cytometry testing. Lack of immunogenicity was confirmed in the final product via MLR testing. Growth factors responsible for osteoinduction were confirmed in the final product via immunoassay testing. Product density was confirmed for proper packaging within the process. Additionally, final product thawing and post-thaw stability were assessed to support the IFU generation for the end user. Based on the testing performed, Production will validate their production process. Additional development testing will be performed to generate data that will inform marketing materials and further process improvements for the product.

### Example 31: Process Parameters

A non-limiting example process parameter is summarized in the below Table 33.

TABLE 33
Non-limiting Example Process Parameters

| Step | Parameter | Value |
|---|---|---|
| Intervertebral | Intervertebral Discs Mass | ≥40 g and ≤100 g |
| Discs | | |
| Decontamination | 70% IPA Volume | 300 mL |
| | Shaker Speed & Duration | 125 RPM for 15 min |
| Washes | Number of Washes | 3 |
| | Plasma-Lyte A Volume | 300 mL |
| | Shaker Speed & Duration | 125 RPM for 1 min (Wash #1 & 2) |
| | | or 5 min (Wash #3) |
| Homogenization | Plasma-Lyte A Volume | 1.5X of Intervertebral Disc Mass |
| | 20% DMSO Wash Medium | 1X of (Intervertebral Disc Mass + |
| | Volume | Plasma-Lyte A Volume) |
| | Number of Blend Cycles | 3 |
| | Blend Cycle Blender Speeds & | Low for 30 sec, Medium for 30 sec, |
| | Times | High for 90 sec, and Rest for 30 to |
| | | 60 sec |
| Bone Grindings | Bone Grindings Mass | ≤500 g |
| Regrind | Regrind Size | XXXF (0.5 to 1 mm) |
| | Rinse Medium Volume | 1X of Bone Grindings Mass |
| Treatment with | Antibiotic/Antimycotic | Vancomycin: 0.5 mg/mL |
| Antibiotics/ | Concentrations in Treatment | Voriconazole: 0.1 mg/mL |
| Antimycotic | Medium | Polymyxin B: 0.1 mg/mL |
| | | Gentamicin: 0.1 mg/mL |
| | Treatment Medium Volume | 1X of Bone Grindings Mass |
| | Shaker Speed & Duration | 125 RPM for 60 min |
| Washes | Number of Washes | 5 |
| | Plasma-Lyte A Volume | 2X of Bone Grindings Mass |
| | Shaker Speed & Duration | 150 RPM for 1 min (Wash #1, 2, 3 |
| | | & 5) or 20 min (Wash #4) |
| Combined Tissue | Tissue Composition | 85% Bone Grindings and |
| | | 15% Intervertebral Discs |
| Treatment with | IRI Concentration in 10% | 2.86 mg/mL |
| Cryoprotectant | DMSO Wash Medium | |
| | 10% DMSO Wash Medium | 1X of Combined Tissue Mass |
| | Volume | |
| | Shaker Speed & Duration | 150 RPM for 10 min |
| | Centrifuge Speed & Duration | 1000× g for 5 min |
| Homogenization | High Torque Stirrer Speed & | 1000 RPM for ≥3 min |
| | Duration | |
| Package & Freeze | Freezer Temperature | ≤−70° C. |
| | Initial Freeze Duration | ≥12 hours |

The main sources of process variation can include:

Masses of Tissues: The masses of intervertebral discs and bone grindings available for VBX production are variable due to donor size and intervertebral disc condition based on OPO recovery, donor health, and/or debridement.

Homogeneity of Tissues: Variability in the homogeneity of the final product can result from donor variability and variability in the homogenization of intervertebral discs, the regrind of bone grindings, and the combining of tissues.

Aliquoting Final Product: Aliquoting the VBX into final product packaging utilizes a manual scooping method, which introduces operator variability.

Packaging Time: The variable masses of tissues result in a variable number of final product units to be packaged, thus resulting in variable packaging times (time between treating the combined tissue with cryoprotectant and transferring the packaged units into the freezer).

In some embodiments, steps in the methods may include preparation & combination of tissues, decontamination and treatment with antibiotics/antimycotic, washes, and/or packaging. Details for each are as follows.

### Preparation & Combination of Tissues

Intervertebral Disc Inspection: To control the quality of the intervertebral discs used, operators are trained on how to inspect and isolate healthy discs for processing (specifically, the nucleus pulposus and anulus fibrosus). In addition to removing any disc degeneration, the cartilaginous endplates are removed due to their hardness. Removing these sections yields a higher quality and more consistent final product. In addition to the initial disc inspection, a visual inspection of the homogenized discs is performed, which helps to identify and remove any unblended cartilaginous endplates not previously removed.

Intervertebral Disc Homogenization: To control variability in the homogenization of intervertebral discs, a minimum of 40 g and a maximum of 100 g discs are processed. For blending, the discs are combined with solution at a set ratio and a specific blending protocol is followed. Following blending, a visual inspection is performed to remove any pieces larger than or equal to 0.5 cm in the disc suspension. This same inspection is performed again after the tissues have been combined just prior to final product packaging.

Bone Grindings Regrind: To control variability in the regrind of bone grindings, a maximum of 500 g grindings is processed. The same milling drum size is utilized along with a set ratio of medium relative to the mass of bone grindings. Additionally, a visual inspection of the reground bone grindings is performed to remove any pieces larger than or equal to 0.5 cm and/or red in color. This same inspection is performed again after the tissues have been combined just prior to final product packaging.

Combination of Tissues: To control variability in the combining of tissues, the disc suspension and reground grindings are combined in a set ratio. Additionally, a high torque stirrer is utilized to mix the final product prior to packaging, minimizing operator variability.

Decontamination and Treatment with Antibiotics/Antimycotic

Intervertebral Disc Decontamination: To control variability in the decontamination of intervertebral discs, a minimum of 40 g and a maximum of 100 g discs are processed. As a result, the decontamination with 70% IPA is performed with a volume at least three times the disc mass.

Bone Grindings Treatment with Antibiotics/Antimycotic: To control variability in the antibiotics/antimycotic treatment of bone grindings, a maximum of 500 g grindings is processed. The bone grindings are submerged in a volume of Treatment Medium relative to the mass of bone grindings. This ensures the quantities of antibiotics/antimycotic are scaled proportionally to the quantity of bone grindings.

### Washes

Intervertebral Discs: To control variability in the effectiveness of the post-decontamination washes, a minimum of 40 g and a maximum of 100 g discs are processed. As a result, the Plasma-Lyte A washes are performed with a volume at least three times the disc mass.

Bone Grindings: To control variability in the effectiveness of the post-treatment washes, a maximum of 500 g is processed. Furthermore, the Plasma-Lyte A washes are performed with a volume relative to the mass of bone grindings.

### Packaging

Aliquoting Final Product: To control variability in fill volumes, the final product units are aliquoted by mass. Each unit is weighed during filling to ensure each aliquot meets the minimum 1.3 g per cc. Additionally, setting a maximum weight (e.g., 0.5 g above the minimum) maintains a narrow range. Furthermore, each weight is verified by a second operator.

Packaging Time: To control variable final product packaging times, an upper limit is defined. To maintain consistency with the VBX process, a maximum 3-hour packaging time is acceptable as this range was demonstrated to not negatively impact the product's quality attributes.

### Impact of Variation on Product and Quality Attributes

The quality attributes are sterility and container closure and labeling, both ensuring product safety. These attributes and their acceptance criteria are presented in Table 34. These quality attributes are part of the lot-release criteria, along with other criteria outside the scope of process design. Execution of the VBX production process as defined has demonstrated reproducible final product sterility and container closure and labeling.

TABLE 34
Summary of Quality Attributes for Processing

| Quality Attribute | | Acceptance Criteria |
|---|---|---|
| Safety | Sterility | No Growth |
| | Container Closure and Labeling | Pass |

There is no quality attribute nor lot-release acceptance criteria for potency because even though the tissue is processed and frozen in a manner that preserves native cells in the final product, the metabolic activity of cells is not an indicator of the product's clinical efficacy. Furthermore, the presence of viable cells, in addition to growth factors, within the final product was reproducibly demonstrated during developmental testing and will continue to be characterized in future clinical lots.

Sterility: A deviation in the Treatment Medium composition and/or treatment time (reduced concentrations of antibiotics/antimycotic and/or reduced treatment time) could potentially impact final product sterility. Similarly, a deviation, specifically a reduction, in the intervertebral disc 70% IPA decontamination volume and/or time could impact sterility.

Container Closure and Labeling: A deviation in the heat sealer temperatures and/or time could potentially impact final product container closure and labeling. In the case of container closure, the risk has a potential impact on sterility.

The heat sealer settings may vary as they are specific to the heat sealer being used and the packaging material being sealed; therefore, these parameters are outside the scope of process design.

Handling: A deviation in the homogenization of intervertebral discs, regrind of bone grindings, and/or combining of tissues could potentially impact the final product's handling properties for the end user.

Immunogenicity: A deviation in the number of washes, specifically a reduction in the number of washes, could potentially impact the final product's immunogenicity. However, as little as three washes still demonstrated no immunogenic response for VBX.

Cell Viability & Growth Factors: A deviation in any of the control points could potentially result in decreased cell viability and/or growth factors in the final product.

### Quality Control Testing

Package Inspection & Sterility: For each clinical lot of VBX produced, every final product unit is visually inspected by QC to confirm correct labeling, intact packaging and seals, and no visible foreign objects or contamination. Additionally, a minimum of 10% of the final product mass is packaged for external sterility testing to confirm final product sterility. The release of each lot of VBX is contingent on all units passing the package inspection (container closure and labeling) and the 10% of final product mass passing sterility testing, to ensure final product safety.

B/F Method Validation: Prior to the first performance of final product sterility testing, representative final product units are shipped for external method validation testing. The external test method to determine VBX sterility must be successfully validated prior to assessing a VBX lot for bacterial and fungal growth.

A summary of non-limiting example decisions on production processing is as shown in the below Table 35.

TABLE 35
Non-Limiting Example of Process Decisions

| Process Step | Decisions |
|---|---|
| Intervertebral Discs | 1. Decontaminate intervertebral discs with 70% IPA. |
| | 2. Perform 3 washes post-decontamination. |
| | 3. Homogenize intervertebral discs with 4X the volume of medium |
| | relative to mass of discs. |
| | 4. Place ice packs around blending container to accelerate cooling of |
| | disc suspension. |
| Bone Grindings | 1. Regrind bone grindings with XXXF milling drum. |
| | 2. Treat bone grindings with the same antibiotics/antimycotic as VBX. |
| | 3. Perform 5 washes post-treatment. |
| Combined Tissue | 1. Combine 85% reground bone grindings and 15% intervertebral discs. |
| | 2. Treat combined tissue with 10% DMSO and IRI. |
| | 3. Centrifuge at 1000× g utilizing 500 mL centrifuge tubes to decant |
| | disc-containing tissue. |
| | 4. Use high torque stirrer to mix final tissue product. |
| | 5. Use final product density of 1.3 g per cc for aliquoting by mass. |

### Example 32: Stability of Growth Factors

The concentration of FGF-1, BMP-7, and osteoactivin, was monitored over three months in two compositions-Composition A and Composition B. Composition A was prepared using the process as disclosed previously, such as, for example, in US Patent App. No. US20240164371A1, which is incorporated herein by reference in its entirety. Composition B was prepared as described in various embodiments described herein, including, for example, in Table 33 and FIG. 6. A summary of non-limiting example concentrations are as shown in the below Table 36.

TABLE 36
Non-Limiting Example Concentrations
of Growth Factors over Time

| | Growth | 0 months (pg/gram | 3 months (pg/gram |
|---|---|---|---|
| Composition | Factors | of allograft tissue) | of allograft tissue) |
| Composition | FGF-1 | 230.013 ± 103.14 | 263.79 ± 134.40 |
| A | BMP-7 | 644.71 ± 49.98 | 494.24 ± 76.81 |
| | osteoactivin | 551.02 ± 324.39 | 1802.54 ± 273.25 |
| Composition | FGF-1 | 225.90 ± 35.26 | 198.88 ± 15.54 |
| B | BMP-7 | 1620.42 ± 633.98 | 751.43 ± 222.02 |
| | osteoactivin | 1106.20 ± 216.32 | 1067.56 ± 123.44 |

As shown above, the processing of Composition B results in much higher initial BMP-7 and osteoactivin levels compared with the processing for Composition A. After three months, the osteoactivin levels are essentially the same in Composition B, whereas they increase dramatically in concentration for Composition A.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above and elsewhere herein without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above and elsewhere herein. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

What is claimed is:

1. A composition comprising bone grinding and a tissue sample derived from intervertebral disc, wherein the tissue sample derived from intervertebral disc comprises at least about 10% of the mass of the composition.
